(19) United States
(12) Patent Application Publication
(10) Pub. No.: US 2022/0016196 A1
(43) Pub. Date: Jan. 20, 2022

(54) Aloe Based Compositions Comprising Polysaccharides and Polyphenols for Regulation of Homeostasis of Immunity

(71) Applicant: Unigen, Inc., Tacoma, WA (US)

(72) Inventors: Mesfin Yimam, Tacoma, WA (US); Ping Jiao, Newcastle, WA (US); Teresa Horm, Renton, WA (US); Lidia Brownell, Tacoma, WA (US); Mei Hong, Tacoma, WA (US); Alexandria O'Neal, Orting, WA (US); Qi Jia, Olympia, WA (US)

(73) Assignee: Unigen, Inc., Tacoma, WA (US)

(21) Appl. No.: 17/370,200

(22) Filed: Jul. 8, 2021

Related U.S. Application Data
(60) Provisional application No. 63/049,871, filed on Jul. 9, 2020.

(51) Int. Cl. A61K 36/886; A61K 36/076; A61K 36/53; A23L 33/105; A61K 45/06

(52) U.S. Cl. CPC A61K 36/886; A61K 36/076; A61K 2236/37; A23L 33/105; A61K 45/06; A61K 36/53

(57) ABSTRACT

Compositions used and methods are disclosed for regulation of immunity homeostasis including a combination of an Aloe extract enriched for one or more polysaccharides; a Poria extract enriched for one or more polysaccharides; and a Rosemary extract enriched for one or more polyphenolic compounds. Compositions for maintenance of immunity homeostasis by regulating HMGB1, comprising a combination of one or more polysaccharides and one or more polyphenolic compounds are disclosed. Methods for treating, managing, promoting regulation of immunity homeostasis in a mammal are disclosed that include administering an effective amount of a composition from 0.01 mg/kg to 500 mg/kg body weight of the mammal.

This United States Utility Patent Application is based on U.S. Provisional Patent Application Ser. No. 63/049,871 filed on Jul. 9, 2020 and entitled “Aloe-Based Compositions Comprising Polysaccharides and Polyphenols for Regulation of Homeostasis of Immunity”, which is commonly owned and incorporated herein in its entirety by reference.

### BACKGROUND

Aloe barbadensis M. (Aloe vera), a member of Liliaceae family, has been used as a food or a topical gel, as well as folk medicine for centuries. The oldest medical record of aloe can be dated back to 2200 BC as aloe plants were known to have great healing power. Today, the well-documented beneficial effects, such as wound healing acceleration, anti-microbial effects, anti-inflammatory effects, skin protection, hair growth stimulation, and immuno-stimulating properties and so on, make Aloe vera an important ingredient in nutraceuticals and cosmetics, with many applications after formulation into foods, beverages, dietary supplements, skin care products, etc. (Wynn et al., 2005; Djuv and Nilsen, 2012; Shimpo et al., 2002).

Among the various aloe constituents, acetylated polysaccharides (ACP) are considered to be one of the most important active components. Although there is considerable discrepancy with regard to the structure, chemical and physical properties of the polysaccharides, the major polysaccharides of aloe gel are reported as acetylated mannan (acemannan, ACM or AP), which consists of linear chains of β-1,4-linked mannose substituted with O-acetyl groups having molecular weight in a range of from 3,000 Da to 2,000,000 Da. Aloe polysaccharides have been reported to have strong antioxidant capacity. For instance, strong antioxidant activity has been reported for purified polysaccharides from Aloe barbadensis gel when tested in DPPH, hydroxyl and alkyl radical scavenging assays (Kang et al., 2014). Similarly, in an Aloe plant age and function-related study, polysaccharides from three-year-old aloe leaf extract were found to show the strongest radical scavenging activity (72.19%) which was significantly higher than that of the synthetic antioxidants butylated hydroxytoluene (70.52%) and a-tocopherol (65.20%) at the same concentrations of 100 mg/L via DPPH assay (Hu et al., 2003). Polysaccharides isolated from A. vera have also been found to possess high antioxidant efficacy as demonstrated by a decrease in the oxidative stress biomarker malondialdehyde (MDA) and increases in the hepatic non-enzymatic antioxidant GSH and enzymatic antioxidant SOD in vivo in chronic alcohol-induced hepatotoxicity in mice (Cui et al., 2014).

Aloe vera has been clinically studied for skin aging, skin protection, wound healing, gingivitis, cancer treatment, diabetes treatment, enhancement of bioavailability of vitamin C and vitamin E, for the treatment of pre-diabetes, irritable bowel syndrome, liver protection, treatment of stomach and mouth ulcer. Not many immune-related human clinical trials have been conducted, even though many in vitro and in vivo studies showed immune protection or stimulative effects from Aloe vera leaf extracts and from Aloe polysaccharides. Aloe polysaccharides reduced IL-10 in skin cells after ultraviolet radiation (Byeon et al, 1998) and orally and topically administered Aloe vera gel and purified polysaccharides with molecular weight between 80-200 KDa restored animal's skin immune function that was suppressed by UV exposure (Qiu, et al. 2000, Im, 2005). Oral administration of Aloe polysaccharides significantly reduced the growth of C. albicans in the spleen and kidney following intravenous injection of C. albicans in normal mice (Im, 2010). Aloe polysaccharides also increased cytokine production, including IL-2, IL-4, IL-6, IL-12, IFN-γ and GM-CSF in Peyer's Patch cells from Endoxan-treated mice (Im, 2014). In a cellular model (Budai 2013), Aloe vera significantly reduced IL-8, TNFα, IL-6 and IL-1β cytokine production in a dose dependent manner. The inhibitory effect was substantially more pronounced in primary cells. Aloe vera inhibited the expression of pro-IL-1β, Nlrp3, caspase-1 and the P2X7 receptor in the LPS-induced primary macrophages, reduced inflammasome activation. Furthermore, LPS-induced activation of signaling pathways like NF-κB, p38, JNK and ERK were inhibited by Aloe vera in these cells. Modified Aloe Polysaccharide (MAP) restored chronic stress-induced immunosuppression in mice by restoring the proliferative activities of lymphocytes; ovalbumin (OVA)-specific T cell proliferation; antibody production; and the cell killing activity of cytotoxic T lymphocytes (Lee 2016).

Poria cocos Wolf, a fungus in the family Polyporaceae, is a medicinal mushroom growing on the roots of Chinese red pine trees and other conifers, with common names such as Fuling ( [symbol] ) [symbol] ) in China, and matsuhodo in Japan and also known as hoelen, poria, tuckahoe, or China root.

Its Latin nomenclature has been revised several times, with Wolfiporia extensa as the current botanical name. Fuling as a dual usage ingredient in food and Traditional Chinese Medicine (TCM) in China, has been included in many ancient decoctions and formulas, which are still widely used even today. The property of Fuling is defined as diuretic, sedative and tunic. The traditional usage of Fuling is for treating nausea, vomiting, diarrhea, loss of appetite, and stomach ulcer as well as insomnia and amnesia (Rios 2011; Feng et al. 2013). Many biological activities have been reported for this fungus and fungal extracts, including anti-microbial, anti-fungal, antioxidant, neuroprotective, anti-inflammatory, anti-angiogenic and anti-cancer activities.

The major active constituent of fuling is Poria cocos polysaccharides (PCP), in the form of β-glucan, which is the major component of the dried fungal fruit body, with a molecular weight range from 41 KDa to 5 MDa. Glucose, fucose, arabinose, xylose, mannose and galactose are detected in PCP, with β-(1→3)-linked glucose backbone and β-(1→6)-linked glucose side chains. Variable biological functions have been reported for Poria cocos polysaccharides, such as antioxidant, anti-hyperglycemic, stomach pain alleviation, anti-inflammation, anti-cancer and immunological modulation (Sun 2014). Polysaccharides were reported to have anti-tumor activities against different cancers on both in vivo and in vitro models. Poria cocos polysaccharides has been shown to reduce ox-LDL-induced inflammation and oxidative stress in vascular smooth muscle cells (VSMCs). PCP significantly attenuated ox-LDL-induced oxidative stress, as evidenced by decreased reactive oxygen species (ROS) and MDA levels, and increased SOD activity in VSMCs. PCP also substantially inhibited VSMCs foam cell formation and intracellular lipids accumulation. A mechanism of action study suggested PCP might activate the ERK1/2 signaling pathway, increase Nrf2 translocation from cytoplasm to nucleus and increase heme oxygenase-1 (HO-1) expression, with potential as a therapeutic agent for treating atherosclerosis.

Triterpenoids, which are being researched for anti-cancer, anti-inflammatory, and potential immunological functions, were also identified as active components in Poria cocos, (Rios 2011; Li et al. 2011). Most of the triterpenes isolated from Poria derived from lanostane or secolanostane skeletons. Although the anti-inflammatory mechanism of Poria cocos is not fully understood, inhibition of the phospholipase A enzyme has been confirmed by several studies (Rios 2011; Giner-Larza et al. 2000). The mechanism of the anti-inflammation of P. cocos ethanol extracts was shown to be through inhibition of iNOS, COX-2, IL-1β, and TNF-α by inactivation of the NF-κB signaling pathway in lipopolysaccharide (LPS)-stimulated RAW 264.7 macrophages (Jeong et al. 2014). The inhibitory effects of Poria cocos extracts and lanostane triterpenes on phospholipase A2 (PLA2) have been clearly demonstrated in different in vitro and in vivo models (Giner-Larza et al. 2000). Poria cocos extract was active against PLA2-induced mouse paw edema given by oral or parenteral. Two lanostane triterpenoids isolated from Poria cocos, pachymic acid and dehydrotumulosic acid, were identified as strong phospholipase A2 inhibitors from snake venom with an IC₅₀ value at 0.845 mM determined for dehydrotumulosic acid (Cuéllar et al 1996). Pachymic acid and hydrotumulosic acid also inhibited acute ear edema induced by tetradecanoyl phorbol acetate (TPA) with IC₅₀ values of 4.7 and 0.68 nmol/ear, respectively. These two compounds acted on carrageenan and arachidonic acid-induced acute edema as well, indicating the potential of these triterpenoids as anti-inflammatory therapeutic agents (Cuellar et al 1997). Various isolated triterpenes from Poria cocos were reported for similar inhibition on ear edema induced by TPA or arachidonic acid (Giner, 2000; Yasukawa, 1998; Kaminaga, 1996)

Poria cocos are commonly included among immunomodulating traditional herbs. Poria cocos 50% ethanol extract increased the secretion of interleukin (IL)-1β and IL-6 in human peripheral blood monocytes in vitro in a dose-dependent manner. The extract could increase cytokine levels, including tumor necrosis factor (TNF)-α after 6 h treatment at 0.4 mg/mL; while suppressing the secretion of transforming growth factor (TGF)-β 3 h after treatment at 0.2 mg/mL (Yu and Tseng, 1996). Because the Poria cocos extract enhances the secretion of immune stimulators (IL-1β, IL-6, and TNF-α) by activated macrophages, while suppressing the immune suppressor (TGF-β), it could serve as an immune stimulating agent. The potential mechanism of Poria cocos polysaccharides (PCPs) might be via the activation of T cells. PCP was tested for its immune-adjuvant activity on a in vivo induction of alloreactive murine cytotoxic T-lymphocytes. The augmented cytotoxic T-lymphocyte (CTL) activity within spleen cells and mesenteric lymph node cells persisted for more than 25 days (Hamuro, 1978). PCPs could significantly improve macrophage phagocytosis, thymus index and spleen index (Zhang et al and Peng et al), and increase levels of IgA, IgG and IgM in serum. A study also showed that the immunomodulatory activity of PCP could be via TLR4/TRAF6/NF-κB signaling, demonstrated both in vitro in RAW 264.7 macrophages and in vivo in Lewis lung carcinoma (LLC) tumors in mice (Tian, 2019). Adjuvants are important components of vaccination strategies because they boost and accelerate the immune response. The adjuvant activity of PCP has been reported with different vaccines in animals including the rabies vaccine and the hepatitis B vaccine, indicating that PCP is an excellent adjuvant candidate for boosting inactive vaccines (Wu, 2016; Zhang, 2019).

Rosemary (Salvia Rosmarinus, Rosmarinus officinalis), is a woody, perennial herb growing up to two meters high. The leaves are evergreen, similar to pine needle with pungent aroma. It's a member of the mint family of Lamiaceae, native to the Mediterranean region and cultivated worldwide in many countries including USA, England, France, Spain, Portugal, Morocco, China, etc. The fresh and dried leaves are used frequently in traditional Mediterranean cuisine as a spice to flavor various foods such as roasted meat. Both the rosemary leaves and the rosemary oil, prepared by distillation from the fresh flowering top or the stems and leaves, can be extensively utilized in foods and beverages including alcoholic beverages, frozen dairy, desserts, baked goods and meat products (Leung and Foster, 1996). Rosemary is one of the oldest known medicinal plants for its astringent, tonic, carminative, antispasmodic, choleretic, mucolytic, analgesic and diaphoretic properties. Throughout the centuries, Rosemary is believed to enhance memory and mental clarity. It could stimulate, rejuvenate and uplift the spirit, mind and body (Zimmermann, 1980; Newall, 1996). Rosemary leaves are approved to treat dyspeptic disorders, blood pressure problems, loss of appetite and rheumatism (PDR for Herbal Medicines, 2nd Ed.). It can be also used as a folk medicine for digestive complaint, headaches and migraine, menstrual problems, exhaustion, dizziness and poor memory.

Rosemary is orally taken for dyspepsia, flatulence, inducing abortion, increasing menstrual flow, gout, cough, headache, liver and gallbladder problems, loss of appetite, and for cardiovascular conditions such as high blood pressure (Natural Medicines Comprehensive Database, 2010). Rosemary is traditionally used in herbal medicine as a homeopathic remedy to help relieve muscle and joint pain associated with rheumatism (Leung and Foster, 1996; ESCOP 2003). It could help improve circulation, which is beneficial for muscle tension and rheumatism. Rosemary aromatherapy may also relieve headache, reduce stress and aid in lessening asthma and bronchitis symptoms. Rosemary is reported for antimicrobial, antifungal, and antiviral activities (Newall, 1996). The powdered leaves are used as an effective natural flea and tick repellent. Rosemary oil showed significant antibacterial, antifungal, and antiviral properties as well. Studies have reported Rosemary's antioxidant activity (Al-Sereiti, 1999). Caffeic acid, Rosmarinic acid and phenolic diterpenes carnosic acid and carnosol were the compounds associated with the antioxidant properties of Rosemary extracts.

Rosmarinic acid (RA) is a water-soluble caffeoyl phenolic acid compound, an ester composed of caffeic acid and 3-(3,4-dihyroxyphenyl) lactic acid. Rosmarinic acid has been reported as one of the principle components in Rosemary and Salvia species with a wide range of biological activities, mainly antioxidant, antimicrobial, antiviral, anticancer, anti-apoptotic, and anti-inflammatory effects, etc. Strong antioxidant activities in 14 Salvia plants species were correlated with Rosmarinic acid contents (Adimcilar, 2019). Rosmarinic acid and its two metabolites Caffeic acid and 3-(3,4-dihyroxyphenyl) lactic acid all showed potent free radical-scavenging activity comparable to the positive control, quercetin, in both non-cellular and cellular antioxidant assays (Adomako-Bonsu, 2017).

The anti-inflammatory effects of Rosmarinic acid (RA) have been studied in different in vitro and in vivo models with its potential usage in a series of inflammatory diseases, like arthritis, colitis, asthma, and allergic rhinitis (Amoah, 2016; Luo, 2020). RA was found to inhibit IL-6 secretion and inhibit gene expression and protein levels of ADAMTS-4 and ADAMTS-5 in IL-1β-induced rat chondrocytes (Hu, 2018). In this study, RA also reduced ACAN and COL2 gene expression potentiating its usage in treating osteoarthritis. RA was reported to inhibit ovalbumin (Ova)-stimulated airway inflammation in a mouse model of asthma (Liang, 2016). RA significantly reduced inflammatory cells and Th2 cytokines in bronchoalveolar lavage fluid (BALF), decreased total IgE and Ova-specific IgE concentrations, and significantly improved airway hyperresponsiveness. Pretreatment with RA could significantly decrease AMCase, CCL11, CCR3, Ym2, and E-selectin mRNA levels in lung tissue and reduce NF-kB and MAPK activation, indicating that RA may be a promising candidate for asthma treatment, potentially through the inhibition of ERK, JNK, and p38 phosphorylation and through the inactivation of NF-kB. Oral RA was found effective in a 12-tetradecanoylphorbol 13-acetate (TPA)-stimulated mice ear edema model (Osakabe, 2004), markedly reducing the number of neutrophils and eosinophils in nasal lavage fluid.

Antisepsis effects of Rosmarinic acid (RA) were investigated in cultured RAW264.7 macrophage-like cells and in a sepsis model induced by cecal ligation and puncture in rats (Jiang, 2009) with decreased local and systemic levels of a broad spectrum of inflammatory mediators. RA down-regulated the levels of TNF-α, IL-6, and high-mobility group box 1 protein (HMGB-1) in a dose-dependent manner. The anti-inflammatory mechanism of RA may be via modulation of the NF-κB pathway by inhibiting IKB kinase activity. RA showed significant downregulation of the pro-inflammatory gene cyclooxygenase-2 (COX-2) in both the colon cancer HT-29 cell line, and in nonmalignant breast epithelial cell line MCF10A (Scheckel, 2008). An antiviral effect of RA was reported in mice infected with Japanese encephalitis virus with reduced mortality in the RA-treated group (Swamp, 2007). RA could significantly decrease viral loads and proinflammatory cytokine levels, particularly in IL-6 and 12, TNF-α, IFN-γ, and MCP-1, compared to the levels of infected animals without treatment.

Rosmarinic acid had demonstrated anti-tumor effects on hepatocellular carcinoma (HCC) in the H22-xenograft model by inhibiting the inflammatory cytokines and NF-κB pathway, reducing inflammatory signagling in the tumor microenvironment (Cao, 2016). RA effectively inhibited tumor growth through regulating the ratio of CD4+/CD8+ T cells and the secretion of IL-2 and IFN-γ, reducing the expression of IL-6, IL-10 and STAT3, up-regulating Bax and Caspase-3, and down-regulating Bcl-2. These activities implicated RA in the regulation of the immune response and induction of HCC cell apoptosis (Cao, 2019).

Lipopolysaccharide (LPS) is an integral component of the outer membrane of gram-negative bacteria and a major contributing factor in the initiation of a generalized inflammatory process that could lead to endotoxic shock. Sepsis may lead to life-threatening organ dysfunction, caused by a dysregulated host response to infection and leading to organ failure. It is a state mediated principally by macrophages/monocytes and is attributed to excessive production of several early phase cytokines such as TNF-α, IL-1, IL-6 and IFN-γ, as well as late stage mediators, such as HMGB1. High-mobility group box protein 1 (HMGB1), is a critical mediator of sepsis. It is released from activated macrophages and monocytes in response to endogenous and exogenous inflammatory signals (Wang et al., 1999). Over activity of immune signaling could lead to a cytokine storm which may result in multiple organ failure and ultimately death. Surviving patients could have an ongoing inflammatory response that may well be driven by the late and continued release of HMGB1 (Gentile and Moldawer, 2014).

Once released actively from stimulated mononuclear cells and passively from necrotic cells, HMGB1 acts as an alarmin (danger signal) serving to activate the host immune response. It plays a critical role in activation of the innate immune response, by functioning as a chemokine facilitating movement of immune cells to sites of infection, and as a damage-associated molecular pattern (DAMP), activating other immune cells to secrete pro-inflammatory cytokines (Yang et al., 2001). When pro-inflammatory cytokines are produced at low and optimum levels, they will yield a protective immune response against viral or bacterial invasion; However, if they are overproduced as in the case of a ‘cytokine storm’, they become harmful to the host by mediating an injurious inflammatory response. In most cases, for subjects with underlying health conditions, with immunodeficiency or compromised immunity and in the elderly, cytokine storms seem to cause acute systemic inflammatory syndrome; those who survive may develop a delayed mediation of inflammation which could result in persistent inflammatory, immunosuppressive and/or catabolic responses. Besides serving as a chemoattractant for a number of cell types, including all inflammatory cells, HMGB1 causes inflammatory cells to secrete more TNF-α, IL-1β, IL-6, IL-8, and macrophage inflammatory protein (MIP) suggesting its participation in a ‘cytokine storm’ (Bianchi and Manfredi, 2007). Significant studies have also reported extracellular HMGB1 can trigger a devastating inflammatory response and promotes the progression of sepsis and acute lung injury (Entezari et al., 2014). In contrast to TNF-α and IL-1β, which are secreted within minutes of endotoxin stimulation, HMGB1 is secreted after several hours, both in vitro and in vivo indicating its late-stage inflammatory mediation. In fact, when HMGB1-neutralizing antibodies administered 24hr after the onset of sepsis, provided protection against lethal endotoxemia indicating the key role of HMGB1 as a late mediator of lethal sepsis (Wang et al., 1999). Clinically, a strong association had also been established between persistently high level of HMGB1 and subjects in the late stage of sepsis or who succumbed from sepsis (Angus et al., 2007).

Aging is a complicated degenerative process that affects both the body and mind function over time, and poor immune response is one of the most observed changes in the senile. Understanding the underlying mechanisms in the decline of the immune response that occurs in the elderly is a key first step in its mitigation. Chemically-induced accelerated aging models, such as the D-galactose induced thymus damage and immune senescence mouse model, are preferred options to study the impacts of aging on the immune system. In the chemically induced animal aging models, animals exhibit immunosenescence that mimics a decline in immune response frequently observed in the elderly (Azman 2019). D-galactose induced aging model is one of the commonly used and well-validated animal models in anti-aging research. While it is converted to glucose at normal concentrations in animal body, high concentrations of D-galactose could easily be converted to aldose and hydroperoxide, leading to production of oxygen derived free radicals. It could also react with free amines of protein and peptides to produce advanced glycation end products (AGEs) through non-enzymatic glycations. Accumulation of these reactive oxygen species (ROS) and increased AGEs in this model would result in disequilibrium of normal organ and immune system homeostasis, which subsequently could cause oxidative stress, inflammation, decreased immune response, mitochondrial dysfunction, and apoptosis (e.g. of thymus cells) that ultimately accelerates the aging process. These changes are among the naturally occurring pathological characteristics of senescence and aging.

The contemplated subject matter describes a novel Aloe-based composition comprising polysaccharides and polyphenols for regulation of immunity homeostasis. With respect to the current contemplated subject matter, achieving homeostasis of immunity has been approached from two separate response triggers to the host defense mechanisms. For simplicity, these response triggers were categorized based on their origins of assault as endogenous/intrinsic and exogenous/extrinsic. While exposure to pollution, infection, chronic diseases, and any foreign invasion fall under the category of exogenous origin; inflammation, oxidative stress, stress hormones, aging and its associated changes are classified under endogenous origins in this contemplated subject matter. Regardless of the cause, recovery, protection and/or prevention from any of the disclosed endogenous and/or exogenous assault triggers depends on the capacity of the host immune response to restore homeostasis.

In some embodiments, contemplated methods include maintaining immune homeostasis by optimizing or balancing the immune response; improving aging and immune organ senescence compromised immunity; preventing chronic inflammation and inflammation-compromised immunity; helping to maintain a healthy immune response to influenza vaccination or COVID-19 vaccination; helping to maintain a healthy immune function against virus infection and bacterial infections; protecting the immune system from oxidative stress damage induced by air pollution of a mammal.

The Aloe-based novel composition, UP360 comprising polysaccharides and polyphenols, described in the current contemplated subject matter, was shown to address both the endogenous and exogenous assault scenarios. While the lipopolysaccharides (LPS)-induced sepsis, LPS-induced acute lung injury, hyperoxia and microbial infected mouse model and immunization models were used to imitate the exogenous impact, the D-Galactose-induced accelerated aging model with and without immunization was used to mimic the endogenous effect of the contemplated composition. In both cases, the current contemplated subject matter showed statistically significant improvement in immune responses of the host, suggesting the positive drive of the novel composition to restore homeostasis. Efficacy of the contemplated composition was assessed based on the changes observed in key immune and/or inflammatory response biomarkers, such as HMGB1, and changes associated with immunosenescence. By modulating HMGB1, the Aloe-based composition, UP360 comprising polysaccharides and polyphenols, demonstrated significant mitigation of pro-inflammatory cytokines TNF-α, IL-1β, IL-6, CRP, and CINC3, while increasing the survival rate, indicating its usage as an immune regulator that is able to restore, modulate and maintain homeostasis of immunity. Similarly, the Aloe-based composition, UP360, was also found to show reversal of immunosenescence as evidenced by stimulation of innate and adaptive immune responses (increased IgA, increased CD3+ T cells, CD4+ Helper T cells, CD8+ Cytotoxic T cells, NKp46+ Natural Killer cells, TCRγδ+ Gamma delta T cells, and CD4+TCRγδ+ Helper Gamma delta T cells), augmentation of antioxidant capacity (increased SOD and Nrf2) and preservation of key immune organs, such as thymus, from aging-associated damage.

The novel Aloe-based composition, UP360, which comprises polysaccharides and polyphenols, demonstrated priming and activation of the host immune system for increased immune surveillance and/or a robust response in additional categories such as randomized double-blind placebo controlled human clinical trials. Specifically, TCRγδ+ Gamma delta T cells were increased in subjects after 28 days of daily supplementation with UP360, and in those who took the supplement for 56 days total with an influenza vaccination immune challenge at Day 28. Increased circulating TCRγδ+ Gamma delta T cells are suggestive of heightened immune surveillance in peripheral tissues that have a higher percentage of TCRγδ+ Gamma delta T cells, such as the skin, intestine, and lungs. The merit of combining these standardized and enriched extracts from medicinal plants in the current contemplated subject matter was also tested in the LPS-induced sepsis model in vivo and LPS-challenged macrophages in vitro and unexpected synergistic effects were found as described in the body of the contemplated subject matter. In general, representing the immune system as a lever and the Aloe-based composition comprising polysaccharides and polyphenols as a pivot point, immune homeostasis was achieved by modulating the HMGB1 effect on one side of the lever and gamma delta T-cells counter effect on the other.

### BRIEF DESCRIPTION OF THE FIGURES

FIG. 1 shows the novelty of Aloe-based compositions (UP 360 in this Figure) to maintain homeostasis of immune function by shifting the tipping point—HMGB1.

FIG. 2 shows the H&E stain of lung tissue from LPS induced rats treated with UP360 at 500 mg/kg. A=normal control, B=Vehicle control, C=Sodium Butyrate, D=UP360 (500 mg/kg). Magnification 100×.

### SUMMARY OF THE SUBJECT MATTER

Compositions for regulation of immunity homeostasis are disclosed that include a combination of an Aloe extract enriched for one or more polysaccharides; a Poria extract enriched for one or more polysaccharides; and a Rosemary extract enriched for one or more polyphenolic compounds.

Compositions for maintenance of immunity homeostasis by regulating HMGB1, comprising a combination of one or more polysaccharides and one or more polyphenolic compounds are disclosed, wherein the composition modulates HMGB1 by inhibition of HMGB1 release or counteract its action as targeting HMGB1 active or passive release by blocking cytoplasm translocation, or by blocking vesicle mediated release; or inhibiting intramolecular disulfide bond formation in the nucleus; or targeting HMGB1 directly upon release and neutralize its effect; or blocking HMGB1 pattern recognizing receptors such as Toll-like Receptor (TLR)-2/4/7/9 and receptor for advanced glycation end products (RAGE) or inhibiting their signal transductions; or changing the physiochemical microenvironment and preventing formation of HMGB1 tetramer and interfere the binding affinity of HMGB1 to TLR and RAGE; or preventing cluster formation or self-association of HMGB1.

Methods for treating, managing, promoting regulation of immunity homeostasis in a mammal are disclosed that include administering an effective amount of a composition from 0.01 mg/kg to 500 mg/kg body weight of the mammal.

### DETAILED DESCRIPTION

The natural compounds capable of modulating, suppressing and stimulating any components of adaptive or innate immunity are known as immunomodulators, immunorestoratives, immunoaugmentors, or biological response modifiers. Immunomodulators are generally categorized into immunoadjuvants, immunostimulants, and immunosuppressants in clinical practice. Immunoadjuvants are specific immune stimulators which enhance the efficacy of vaccine. Agents that activate or induce the mediators or components of the immune system are called as immunostimulants. Protection against autoimmunity, cancer, allergy, and infection is enhanced by immunostimulants. On the other hand, immunosuppressants are molecules that inhibit the immune system and can be used to control the pathological immune reaction such as subsequent to organ transplantation.

Maintaining tight immune homeostasis is essential for the physiological function of defense from external invasive microbes, viruses, fungi, pollutants, to clear dead cells, and to initiate rebuild and renewal of respiratory and gastrointestinal function. Overstimulated immune function can cause allergic reactions and auto-immune destructive diseases. Aging, oxidative stress, psychological stress, systemic inflammation, and many chronic diseases such as diabetes, obesity, and metabolic syndrome can shift the homeostasis tipping point leading to compromised immune function. A healthy lifestyle, including daily balanced nutrition, exercise, stress management and supplementation with anti-oxidative, anti-inflammatory and immune regulatory (either immune suppressive and/or immune stimulative, depending on the specific case) natural compounds and prescriptive or OTC drugs for anti-virus, antibiotic, steroids and NTHEs can provide beneficial forces to balance immune function. Through these means, systemic and chronic inflammation can be reduced.

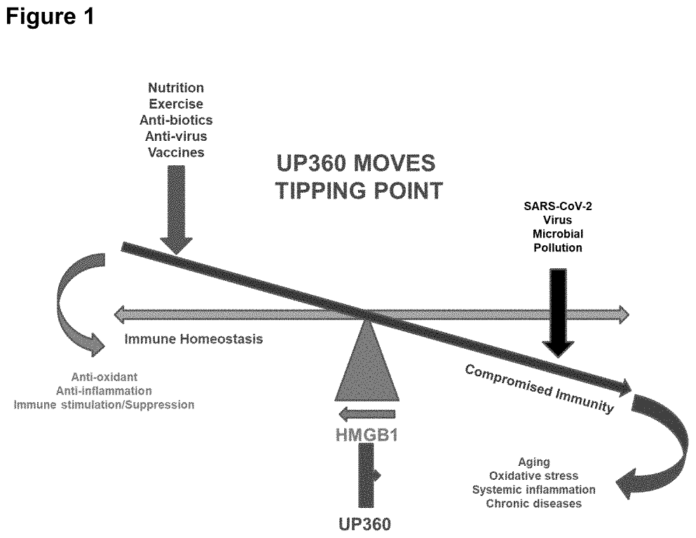

Unfortunately, there is much less knowledge and attention paid to understand whether there are key biological, physiological and pathological pathways and biomarkers that play a critical role as a tipping point factor that, when overactive, can accelerate the shift of the immune response from a healthy level to a downward spiral and lead to a cytokine storm. Finding such a tipping point is important. More essential is finding active compounds to make into a composition that can move the tipping point away from the destructive direction and restore homeostasis of immunity. We believe that HMGB1 is such a biomarker that can act as a tipping point to escalate biological response to virus, such as coronavirus SARS-CoV-2, bacterial infections, and PM2.5 pollutants that lead to compromised and destructive immune responses. The Aloe-based compositions comprising polysaccharides and polyphenols can shift the tipping point by controlling HMGB1 to restore, modulate and maintain homeostasis of immunity (FIG. 1).

HMGB1 was initially identified as a nuclear protein that regulates transcription, by stabilizing the structure of nucleosomes and mediating conformational changes in the DNA. In contrast to its role in the nucleus, extracellular HMGB1 induces significant inflammatory responses. Compiling evidence has shown that the accumulation of high levels of extracellular HMGB1 in the airways can directly compromise host defense mechanisms against bacterial and virus infections via the impairment of macrophage functions in a couple of animal models of pulmonary infections. The levels of nuclear HMGB1 protein is overwhelmingly high (100-fold compared to the healthy controls) in the airways of animals and humans exposed to prolonged oxidative stress. Thus, reducing the levels of HMGB1 in the airways and/or blocking their activities, may provide important therapeutic and preventive strategies for the increasing population subjected to oxidative stress generated by cytokine storm, such as by COVID-19 infection, and those living with inflammatory disorders.

Compositions for regulation of immunity homeostasis are disclosed that include a combination of an Aloe extract enriched for one or more polysaccharides; a Poria extract enriched for one or more polysaccharides; and a Rosemary extract enriched for one or more polyphenolic compounds. In contemplated embodiments and as will be shown in detail herein, the Aloe extract, or Poria extract or Rosemary extract in the composition is in a range of 1%-98% by weight of each extract with the optimized weight ratio of Aloe:Poria:Rosemary (APR) at 3:2:1 (50%:33.3%:16.7%) or 1:1:1 (33.3%:33.3%:33.3%) or 3:6:1 (30%:60%:10%).

In some embodiments, contemplated polyphenolic compounds comprise, and in some embodiments are selected from the group consisting of, Rosmarinic acid, conjugated catechins such as EGCG, ECG, epigallocatechin etc. Oroxylin, Kaempferol, genistein, quercetin, Butein, Luteolin, chrysin, Apigenin, curcumin, resveratrol, capsaicin, glomeratose A, 6-shogaol, gingerol, berberine, Piperine or a combination thereof.

Compositions for maintenance of immunity homeostasis by regulating HMGB1, comprising a combination of one or more polysaccharides and one or more polyphenolic compounds are disclosed, wherein the composition modulates HMGB1 by inhibition of HMGB1 release or counteract its action as targeting HMGB1 active or passive release by blocking cytoplasm translocation, or by blocking vesicle mediated release; or inhibiting intramolecular disulfide bond formation in the nucleus; or targeting HMGB1 directly upon release and neutralize its effect; or blocking HMGB1 pattern recognizing receptors such as Toll-like Receptor (TLR)-2/4/7/9 and receptor for advanced glycation end products (RAGE) or inhibiting their signal transductions; or changing the physiochemical microenvironment and preventing formation of HMGB1 tetramer and interfere the binding affinity of HMGB1 to TLR and RAGE; or preventing cluster formation or self-association of HMGB1. FIG. 1 shows the novelty of Aloe-based compositions (UP 360 in this Figure) to maintain homeostasis of immune function by shifting the tipping point—HMGB1.

Methods for treating, managing, promoting regulation of immunity homeostasis in a mammal are disclosed that include administering an effective amount of a composition from 0.01 mg/kg to 500 mg/kg body weight of the mammal. In some embodiments, contemplated compositions comprise a combination of an Aloe extract enriched for one or more polysaccharides; a Poria extract enriched for one or more polysaccharides; and a Rosemary extract enriched for one or more polyphenolic compounds.

Published and on-going studies have demonstrated that reagents that can attenuate the accumulation of extracellular HMGB1 are effective in improving respiratory function by enhancing innate immunity against air pollutants, bacterial and virus infections and dampening inflammatory responses via improved macrophage function. The model entails subjecting mice to hyperoxia, which is commonly used during oxygen therapy for COVID-19 patients and lung infections, and testing potential treatments to determine whether they can be used as effective tools to achieve better clinical outcomes, including survival, by improving innate immunity and respiratory function and inhibiting the accumulation of extracellular HMGB1 in the airways and in the circulation. The current contemplated subject matter discloses the unique Aloe-based composition comprising polysaccharides and polyphenols that improved innate immunity and alleviated compromised respiratory function by shifting HMGB1 in these models (Examples 12-17). As detailed in Example 51, supplementation with the Aloe-based composition resulted in significant reduction bacterial load in airways and decrease mortality of animals which were exposed to hyperoxia and challenged with pseudomonas aeruginosa indicating its beneficial application in counteracting the effects of hyperoxia and microbial infection.

The contemplated subject matter regulates homeostasis of immunity by targeting the secretion of HMGB1 to the bloodstream, a natural late-stage event in an immune response, by Aloe-based compositions comprising polysaccharides and polyphenols. In vitro, hyperoxic macrophages were treated with the individual constituents of the Aloe based composition and were shown to reduce HMGB1 secretion (Example 12). Since oxidative stress is one of the most potent inducers of HMGB1 release from the cell nucleus, we demonstrated that Aloe composition and its individual components showed reduction of reactive oxygen species (ROS) (Example 13-14) in human keratinocyte induced by UVA and UVB, and protection of hydrogen peroxide-induced DNA damage in human fibroblasts (Examples 15). These cellular assays showed the statistically significant impact of these bioactive compounds extracted from medicinal plants in reducing the HMGB1 level by inhibition of free radical generation and repair of DNA damage, suggesting their standardized formulation for an enhanced outcome for conditions that involve the disclosed mechanisms in a disease pathology. In addition to significantly and synergistically reducing the mortality rate of animals in the LPS induced survival rate studies (Examples 20-22), UP360 also significantly increased the survival rate of animals in hyperoxia induced Pseudomonas aeruginosa infected mice and reduce bacterial load in Airways (Example 51) Key proinflammatory cytokines and chemokines such as IL-1β, TNF-α, IL-6, IL-10, CRP and CINC-3 in conjunction with HMGB1 (Examples 12-31) have been evaluated from in vivo assays and statistically significant reductions of these biomarkers for animals treated with the Aloe-based composition, UP360 comprising polysaccharides and polyphenols, in comparison to the vehicle-treated group as a result of shifting the tipping point, HMGB1, in the homeostasis of immunity.

Objective treatment and response effects were assessed in multiple in vivo studies (such as LPS-induced sepsis models, hyperoxia induced Pseudomonas aeruginosa infected mice survival model and acute lung injury model) as described in the body of the contemplated subject matter (Examples 18-31, 51). Data depicted in the examples of this contemplated subject matter showed significant immune homeostatic effects of Aloe-based compositions when administered orally in septic, hyperoxia induced Pseudomonas aeruginosa infected mice or acute lung injury study subjects. These significant changes in the levels of biomarkers from serum, broncho-alveolar lavage (BAL) and lung homogenates as well as reduced total protein in the BAL demonstrated improved immune homeostasis by shifting HMGB1—the tipping point, and these findings were later confirmed by histology examination. Statistically significant reductions in the overall severity of lung damage and pulmonary edema were observed for animals treated with the Aloe-based composition. An unexpected synergistic effect was also observed when the merit of formulating Aloe-based plant extracts containing two different classes of natural active compounds, immune stimulating polysaccharides and immune suppressing polyphenols, was evaluated in the LPS-induced septic model (Example 22). The data from this current contemplated subject matter indicated that Aloe based compositions consisting of polysaccharides and polyphenols helped maintain homeostasis of immunity by shifting the tipping point—HMGB1. As a result, the Aloe-based composition could be utilized at the time of air pollution, seasonal flu and/or viral and bacterial infections that require a balanced immune response to protect respiratory and lung function from sepsis and/or acute and/or chronic injuries of the respiratory system.

Collectively, data from this study showed that an Aloe-based composition, wherein one of these contemplated compositions may be referred to herein as UP360, has unexpected synergistic activity leading to significant mitigation of acute lung injury induced by intratracheal LPS by counteracting HMGB1, as evidenced by key biomarkers indicative of disease pathology (Examples 23-29). Installation of LPS directly into the lung is known to activate the resident innate immune response via alveolar macrophages releasing a significant amount of HMGB1, leading to increased production of primary cytokines such as TNF-α, IL-1β and IL-6 as well as inflammatory protein CRP. These cytokines can cause significant pulmonary pathology alone or in concert, triggering activation of cascades of cytokines and chemokines detrimental to disease pathology. For example, at the time of acute inflammatory response, the chemotactic cytokine induced neutrophil chemoattractant (CINC-3) plays an important role in the recruitment of neutrophils to the lung in LPS-induced acute lung injury. Suppression of HMGB1, the key tipping point of immune homeostasis, in order to control these major cytokines and chemotactic factors involved in acute inflammatory response in the lung has significant clinical relevance in cytokine storm intervention and alleviating severity of acute respiratory distress syndrome (ARDS).

Proteins and/or fibrin leakage into the interstitial space is a key component in pulmonary edema where increased exudate is an indication of respiratory disease severity. Treatment with aloe-based composition consisting of polysaccharides and polyphenols reduced total protein from the broncho-alveolar lavage (BAL) indicating its significance in alleviating pulmonary pathology (Example 28). These significant changes in the biomarkers from serum, BAL and homogenates have demonstrated that the strategy of administration of the Aloe-based polysaccharide and polyphenol composition led to a statistically significant reduction in the overall severity of lung damage and pulmonary edema that has been confirmed by the histopathology evaluation. Based on the cytokine and histopathology data depicted here, an aloe based composition, in this case UP360, regulated the tipping point of immune homeostasis that led to cytokine storm suppression and mitigation of acute inflammatory lung injury severity.

We exposed mice to D-galactose to induce an immune aging phenotype, treated them with an Aloe-based composition (UP360) comprising polysaccharides and polyphenols at two concentrations, and then introduced the influenza vaccine as an immune challenge and measured immune functions in multiple assays to determine whether a contemplated Aloe-based composition protected immune organs and maintained homeostasis of the immune system (Example 32). The thymus indices for the normal control group and both UP360+D-gal treatment groups were significantly higher than the D-gal group, which demonstrated the protection of this immune organ from senescence by the Aloe-based composition (Example 33 and 35). While only the normal control group had a significantly higher spleen index compared to the D-gal group, the UP360+ D-gal-treated animals showed a positive trend toward protecting the spleen from oxidative stress (Example 34).

We found significant changes in humoral immunity among the immunized groups. The UP360+ D-gal groups had increased serum IgA antibodies compared to the D-gal group (Example 36). This increased level of IgA in the serum indicated that the mucosa achieved a higher level of immune protection because of UP360 treatment.

In measuring the white blood cells in whole blood from the different groups and expressing changes as percentages of cell populations, we found important differences among the immunized mouse groups (Examples 37-42). CD45+ cells (all white blood cells) constituted a higher percentage of live cells in the D-gal group than any other immunized group. CD3+ T cells, CD45+ Helper T cells, NKp46+ Natural Killer cells, and TCRγδ+ Gamma delta T cells were all increased in the immunized UP360+D-gal group compared to the immunized D-gal only group. These data indicated that Aloe based composition UP360 consisting of polysaccharides and polyphenols aided in expansion of immune cell populations, resulting in higher percentages of innate and adaptive immune cells as critical mediators of immune homeostasis.

Expressed as total cell per μL of whole blood, we found profound differences among the non-immunized mouse groups (Examples 43-48). The 400 mg/kg UP360+D-gal group had increased CD3+ T cells, CD4+ Helper T cells, CD8+ Cytotoxic T cells, NKp46+ Natural Killer cells, TCRγδ+ Gamma delta T cells, and CD4+TCRγδ+ Helper Gamma delta T cells than the immune-compromised D-gal only group. These data implied that Aloe-based composition UP360 consisting of polysaccharides and polyphenols primed the inactivated immune system and caused expansion of immune cell populations, increasing immune “readiness” in the non-immunized mice.

Activation and expansion of natural killer cells are key modes of immunomodulation to keep homeostasis of immunity. Natural killer cells are an important component of the innate immune system known to respond quickly to a wide variety of pathological challenges; air pollutants; viral, microbial and fungal infections; and cellular oxidative and hormonal distress, without any priming or prior activation. Natural killer cells perform surveillance of cellular integrity to detect changes in cell surface molecules to deploy their cytotoxic effector mechanism. Natural killer (NK) cells function as cytotoxic lymphocytes and as producers of immunoregulatory cytokines. Following stimulation, NK cells produce large amounts of cytokines, mainly interferon-γ (IFN-γ) and tumor necrosis factor (TNF). These cytokines and others produced by NK cells have direct effects during the early immune response and are significant modulators of the subsequent adaptive immune response, mediated through T cells and B cells. The marked increase in NK cells in the current contemplated subject matter (Examples 41 and 45) as a result of oral administration of a contemplated Aloe-based composition (UP360) consisting of polysaccharides and polyphenols is a clear indication that the contemplated subject matter has a significant impact on innate immunity modulation, suggesting its immediate and effective immune triggering activity is involved in laying a foundation for immune homeostasis.

The human clinical trial of contemplated Aloe-based compositions, including UP360, comprising and, in some embodiments, consisting of polysaccharides and polyphenols also demonstrated an enrichment of specific immune cells in the treated population, before and after an immune challenge (Example 52). Healthy and middle-aged subjects were given daily supplementation with either UP360 or placebo for 28 days before their immune systems were challenged with the influenza vaccine. They continued to take UP360 for an additional 28 days, with immune cell measurements conducted at baseline, after 28 days of treatment, and after 56 days of treatment (28 days post-vaccination). It was found that the Gamma delta (γδ) T cell population was significantly increased at Day 56 (28 days post-vaccination) above both baseline levels, and levels at Day 28. The UP360-treated group had significantly higher circulating Gamma delta (γδ) T cells than the placebo group at Day 56, and the changes in the number of Gamma delta (γδ) T cells from Day 0-Day 56 and from Day 28-Day 56 were significantly higher for the UP360-treated group than the placebo group. Perhaps the most striking primary outcome from this clinical trial preliminary data were the changes observed in these gamma delta T-cells. Through the course of the supplementation, subjects who were given the Aloe-based composition consisting of polysaccharides and polyphenols showed gradual increase in the level of these T-cells moving from day 28 to day 56 where the Aloe-based composition showed 21.5% and 24.5% increase in the percent of TCRγδ+ cells populations at days 28 and 56 post administration, respectively. In contrast, the placebo group showed decreased in these cell population at the same time frame where a 10.5% and 5.6% reduction in the percent of TCRγδ+ cells, were observed at days 28 and 56 post administration, respectively. Compared to Placebo, subjects who received the Aloe-based composition consisting of polysaccharides and polyphenols showed 23.5% and 38.9% increase in the percent of TCRγδ+ cells populations at days 28 and 56 post administration of treatment, respectively. These findings indicated that contemplated Aloe-based compositions, including UP360, comprising and, in some embodiments, consisting of polysaccharides and polyphenols had the ability to bolster the Gamma delta (γδ) T cell population, a cell type that is instrumental in first-line defense against pathogens.

Primarily, the immune regulation, surveillance and homeostasis activities of the current contemplated subject matter have been confirmed by the level of induction observed in the gamma delta (γδ) T cells (both in the clinical and pre-clinical studies) which are known for immune regulation, promoting immune surveillance and immune homeostasis. γδ T cells are a unique T cell subpopulation largely present at many portals of entry in the body, including intestines and lungs, where they migrate early in their development and persist as resident cells. Due to their strategic anatomical locations (mucosal lining of the gastrointestinal and respiratory system), γδ T cells provide a first line of defense based on their innate-like responses in directly killing infected cells, recruiting other immune cells, activating phagocytosis and limiting translocation of pathogens or pollutants to the systemic compartment. These cells are known to undergo rapid population expansion and provide pathogen-specific protection on secondary challenges. Their ideal location in the intestine and respiratory tracks also helps maintain intestinal and respiratory epithelial integrity. Generally, the physiological roles of γδ T-cells include protective immunity against extracellular and intracellular pathogens or pollutants, surveillance, modulation of innate and adaptive immune responses, tissue healing and epithelial cell maintenance, and regulation of physiological organ function. The γδ T-cells share some characteristics with Natural killer (NK) cells as both: are usually considered constituents of innate immunity, recognize transformed/distressed cells, play a prominent role in antiviral protection, facilitate downstream adaptive immune responses and are potent cytolytic lymphocytes. In addition, the γδ T-cells assume the role of antigen presenting cells (Ribot et al., 2021; Bonneville et al., 2010). These rapidly responding immune cells (γδ T-cells and the NK cells) have been induced by contemplated aloe-based compositions, including UP360, comprising and, in some embodiments, consisting of polysaccharides and polyphenols (Examples 42 and 48) in the current contemplated subject matter, leading to enhanced characteristics of these T-cells in achieving immune regulation, surveillance and homeostasis produced by the composition.

We examined antioxidant enzymes and biomarkers in order to surveil antioxidation pathways in the D-galactose induced immune senescence model. The aging phenotype induced by the D-gal model is based on an increase in Advanced Glycation End Products, causing oxidative stress and immune organ damage, similar to the level that would be present in an older animal

(Azman KF, 2019). Increasing antioxidation pathways would counter the detrimental effects of oxidative stress. We found an increase in superoxide dismutase enzyme (SOD) in mouse sera from immunized UP360 (both concentrations) +D-gal groups compared to D-gal alone (Example 49). This indicated that contemplated Aloe-based compositions, including UP360, comprising and, in some embodiments, consisting of polysaccharides and polyphenols enhanced antioxidation pathways that enabled the animals to neutralize free radicals better than the untreated aging animals.

In contemplated subject matter, we also looked at protein levels in the spleens of animals from the immunized groups. The spleen is one of the main organs of the immune system. It contains a high level of white blood cells and controls the levels of immune cell types in the blood. We measured Nrf2, a transcription factor involved in activating antioxidation pathways in response to inflammation and prolonged oxidative stress and found that Nrf2 was significantly increased in spleen homogenates from the UP360+D-gal groups compared to D-gal alone (Example 50).

Altogether, in the D-galactose-induced immune senescence model, we saw significant changes in immune cell populations, anti-oxidative stress pathways, and protection of immune organs in the animals treated with contemplated Aloe-based compositions, including UP360, comprising and, in some embodiments, consisting of polysaccharides and polyphenols that indicated increased immune system priming and activation, which demonstrated a reversion to the phenotype of the normal mice. The thymus indices, serum antibodies, T cells, and Natural Killer cells, and antioxidation factors in the immunized UP360+D-gal groups were higher than the D-gal alone, indicating that the immune systems in the UP360-treated groups were better able to respond to the vaccination than the D-gal group alone. The thymus indices, T cells, and Natural Killer cells in the non-immunized UP360+D-gal groups were higher than the D-gal alone. These data indicated that even in the unchallenged immune system, UP360 primed and activated the immune system, causing expansion of immune cells. These findings demonstrated the ability of contemplated Aloe-based compositions, including UP360, comprising and, in some embodiments, consisting of polysaccharides and polyphenols to aid in activating and maintaining homeostasis of the immune system both during active infections and as a preventive to prime the immune system against infection.

The critical value of combining Aloe, Poria and rosmarinic acid extracts, especially those that have been enriched for specific constituents, was evaluated and confirmed using the commonly used equation (Colby's equation) on data obtained from the LPS induced survival study and LPS challenged macrophages for HMGB1 and TNF-α secretion. With Colby's methodology, a standardized formulation with two or more materials is presumed to have unexpected synergy when the observed value is lower than the expected value (i.e. decreased mortality rate, secretion of HMGB1 and TNF-α), there is an unexpected inhibitory effect. In the current contemplated subject matter, it was intended to confirm contemplated Aloe-based compositions, including UP360, comprising and, in some embodiments, consisting of polysaccharides and polyphenols possesses an unexpected synergy for the decreased mortality rate and unexpected inhibitory effect for the secretion of HMGB1 and TNF-α. As illustrated in Examples 20, an unexpected synergy in decreasing mortality rate was observed from the combination of these extracts for both end point measurements. The beneficial effects seen with treatment using contemplated compositions exceeded the predicted effects observed for each of its constituents at the given ratio. It is only the aloe-based composition achieved the statistical significance for mortality rate reduction after 6 days of LPS challenge. In fact, 36 hours after treatment, there was no animal death was observed for the Aloe-based composition while few animals were found deceased for each of the constituents (i.e. aloe, poria and rosmarinic ccid) administered alone. This fact holds true in that the lowest secretion of HMGB1 and TNF-α from LPS challenged macrophages was observed for the Aloe-based composition than the individual constituents (Example 16 and 17). As detailed in the background of this contemplated subject matter, individually, there are reports regarding the beneficial use of these medicinal plants, however, to the best of our knowledge, this is the first time when these medicinal plants were formulated together to yield contemplated Aloe-based compositions, including UP360, comprising and, in some embodiments, consisting of polysaccharides and polyphenols producing unexpected outcomes such as decreased mortality rate of septic animals and inhibited secretion of HMGB1 and TNF-α from macrophages. These outcomes together with other favorable innate and adaptive immune responses, in particular, the increase in gamma delta T-cells observed in human clinical study and documented in this contemplated subject matter, provide a unique identity to the polysaccharides and polyphenols composition guiding the direction of the host immune response as needed to a balanced stimulatory and/or inhibitory activity resulting an overall immune homeostasis.

In the above and following descriptions, certain specific details are set forth in order to provide a thorough understanding of various embodiments of this disclosure. However, one skilled in the art will understand that the contemplated subject matter may be practiced without these details.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polysaccharide subunits, size or thickness, are to be understood to include any integer within the recited range, unless otherwise indicated. As used herein, the terms “about” and “consisting essentially of” mean±20% of the indicated range, value, or structure, unless otherwise indicated. It should be understood that the terms “a” and “an” as used herein refer to “one or more” of the enumerated components. The use of the alternative (e.g., “and/or”) should be understood to mean either one, both, or any combination thereof of the alternatives. Unless the context requires otherwise, throughout the present specification and claims, the word “comprise” and variations thereof, such as, “comprises” and “comprising,” as well as synonymous terms like “include” and “have” and variants thereof, are to be construed in an open, inclusive sense; that is, as “including, but not limited to.”

Reference throughout this specification to “one embodiment” or “an embodiment” means that a particular feature, structure, composition or characteristic described in connection with the embodiment is included in at least one embodiment of the present contemplated subject matter. Thus, the appearances of the phrases “in one embodiment” or “in an embodiment” in various places throughout this specification are not necessarily all referring to the same embodiment.

The term “prodrug” is also meant to include any covalently bonded carriers, which release the active compound of this disclosure in vivo when such prodrug is administered to a mammalian subject. Prodrugs of a compound of this disclosure may be prepared by modifying functional groups present in the compound of this disclosure in such a way that the modifications are cleaved, either in routine manipulation or in vivo, to the parent compound of this disclosure. Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include acetate, formate and benzoate derivatives of alcohol or amide derivatives of amine functional groups in the compounds of this disclosure and the like.

“Stable compound” and “stable structure” are meant to indicate a compound that is sufficiently robust to survive isolation to a useful degree of purity from a reaction mixture, and formulation into an efficacious therapeutic agent.

“Biomarker(s)” or “marker(s)” component(s) or compound(s) are meant to indicate one or multiple indigenous chemical component(s) or compound(s) in the disclosed plant(s), plant extract(s), or combined composition(s) with 2-3 plant extracts that are utilized for controlling the quality, consistence, integrity, stability, and/or biological functions of the contemplated composition(s).

“Mammal” includes humans and both domestic animals, such as laboratory animals or household pets (e.g., cats, dogs, swine, cattle, sheep, goats, horses, rabbits), and non-domestic animals, such as wildlife or the like.

“Optional” or “optionally” means that the subsequently described element, component, event or circumstances may or may not occur, and that the description includes instances where the element, component, event or circumstance occur and instances in which they do not. For example, “optionally substituted aryl” means that the aryl radical may or may not be substituted and that the description includes both substituted aryl radicals and aryl radicals having no substitution.

“Pharmaceutically or nutraceutically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, vehicle or emulsifier which has been approved by the United States Food and Drug Administration as being acceptable for use in humans or domestic animals.

“Pharmaceutically or nutraceutically acceptable salt” includes both acid and base addition salts. “Pharmaceutically or nutraceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically or nutraceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include the sodium, potassium, lithium, ammonium, calcium, magnesium, iron, zinc, copper, manganese, aluminum salts and the like. In certain embodiments, the inorganic salts are ammonium, sodium, potassium, calcium, or magnesium salts. Salts derived from organic bases include salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2 dimethylaminoethanol, 2 diethylaminoethanol, dicyclohexylamine, lysine, arginine, hi stidine, procaine, hydrabamine, choline, betaine, benethamine, benzathine, ethylenediamine, glucosamine, methylglucamine, theobromine, triethanolamine, tromethamine, purines, piperazine, piperidine, N ethylpiperidine, polyamine resins and the like. Particularly useful organic bases are isopropylamine, diethylamine, ethanolamine, trimethylamine, dicyclohexylamine, choline and caffeine.

Often crystallizations produce a solvate of the compound of this disclosure. As used herein, the term “solvate” refers to an aggregate that comprises one or more molecules of a compound of this disclosure with one or more molecules of solvent. The solvent may be water, in which case the solvate may be a hydrate. Alternatively, the solvent may be an organic solvent. Thus, the compounds of the present contemplated subject matter may exist as a hydrate, including a monohydrate, dihydrate, hemihydrate, sesquihydrate, trihydrate, tetrahydrate and the like, as well as the corresponding solvated forms. The compound of this disclosure may be true solvates, while in other cases, the compound of this disclosure may merely retain adventitious water or be a mixture of water plus some adventitious solvent.

A “pharmaceutical composition” or “nutraceutical composition” refers to a formulation of a compound of this disclosure and a medium generally accepted in the art for the delivery of the biologically active compound to mammals, e.g., humans. For example, a pharmaceutical composition of the present disclosure may be formulated or used as a standalone composition, or as a component in a prescription drug, an over the counter (OTC) medicine, a botanical drug, an herbal medicine, a natural medicine, a homeopathic agent, or any other form of health care product reviewed and approved by a government agency. Exemplary nutraceutical compositions of the present disclosure may be formulated or used as a standalone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

As used herein, “enriched for” refers to a plant extract or other preparation having at least a two-fold up to about a 1000-fold increase of one or more active compounds as compared to the amount of one or more active compounds found in the weight of the plant material or other source before extraction or other preparation. In certain embodiments, the weight of the plant material or other source before extraction or other preparation may be dry weight, wet weight, or a combination thereof. In some contemplated embodiments, polysaccharides are enriched individually and/or in combination by solvent precipitation, ultrafiltration, enzyme digestion, column chromatograph with silica gel, XAD, HP20, LH20, C-18, alumina oxide, polyamide, CG161, and size exclusion column resins. In some contemplated embodiments, one or more polyphenols are enriched individually or in combination by solvent partition, precipitation, distillation, evaporation, ultrafiltration, column chromatograph with silica gel, XAD, HP20, LH20, C-18, alumina oxide, polyamide, size exclusion column and CG161 resins.

As used herein, “major active ingredient” or “major active component” refers to one or more active compounds found in a plant extract or other preparation or enriched for in a plant extract or other preparation, which is capable of at least one biological activity. In certain embodiments, a major active ingredient of an enriched extract will be the one or more active compounds that were enriched in that extract. Generally, one or more major active components will impart, directly or indirectly, most (i.e., greater than 50%, 30% or 20% or 10%) of one or more measurable biological activities or effects as compared to other extract components. In certain embodiments, a major active ingredient may be a minor component by weight percentage of an extract (e.g., less than 50%, 25%, or 10% or 5% or 1% of the components contained in an extract) but still provide most of the desired biological activity. Any composition of this disclosure containing a major active ingredient may also contain minor active ingredients that may or may not contribute to the pharmaceutical or nutraceutical activity of the enriched composition, but not to the level of major active components, and minor active components alone may not be effective in the absence of a major active ingredient.

“Effective amount” or “therapeutically effective amount” refers to that amount of a compound or composition of this disclosure which, when administered to a mammal, such as a human, is sufficient to shift the tipping point of immune homeostasis that leads to the improved immune functions, including any one or more of: (1) stimulated innate immunity (2) enhanced adaptive immunity, especially increase of CD3+, CD4+, CD8+, NKp46+ Natural Killer cells, TCRγδ+ Gamma delta T cells, and CD4+TCRγδ+ Helper Gamma delta T cells (3) suppressed chronic systematic inflammation and oxidative stress (4) protected immune and lung cells from HMGB1 induced cytokine storm damage (5) provided function as potent antioxidant to reduce oxidative stress and increase in superoxide dismutase enzyme (SOD), and Nrf2; (6) maintained homeostasis of innate and adaptive immune responses; (7) enhanced phagocytic index of macrophages in humoral and cell-mediated immune responses; (8) inhibited activation of transcription factors such as NF-kB, NFAT, and STAT3; (9) inhibited lymphocyte activation and pro-inflammatory cytokine genes and/or protein expression (IL-2, iNOS, TNF-α, COX-2, and IFN-γ), (10) reduced level of pro inflammatory cytokines such as HMGB1, IL-1β, IL-6, & TNF-α, (11) down-regulated gene and/or protein expression of HMGB1, COX-2, NOS-2, and NF-κB; (12) inhibited eicosanoids generation by inhibiting phospholipase A2 and TXA2 synthase, COX1, COX2, 5-LOX, 12-LOX, 13-LOX activity; (13) decreased response of Thl and Th17 cells; (14) decreased expression of ICAM and VCAM leading to decreased neutrophile chemotaxis; (15) inhibited MAPKs phosphorylation, adhesion molecules expression, signal transducers and activators of transcription 3 (STAT-3); (16) activated transcription factor NRF2 and induce heme oxygenase-1 (HO-1); and inhibited translocation of HMGB1 from cell nuclear; reduced formation of dimers and trimers of HMGB1 monomer.

Modulation of HMGB1 by the current contemplated subject matter comprising polysaccharides and polyphenols could be inhibition of HMGB1 release and/or counteract its action. HMGB1 modulatory effect of the aloe based composition could be as a result of a) targeting HMGB1 active and/or passive release by blocking cytoplasm translocation, and/or by blocking vesicle mediated release; and/or inhibiting intramolecular disulfide bond formation in the nucleus b) targeting HMGB1 directly upon release and neutralize its effect c) blocking HMGB1 pattern recognizing receptors such as Toll-like Receptor (TLR)-2/4/7/9 and receptor for advanced glycation end products (RAGE) and/or inhibiting their signal transductions. Inhibitions of oxidative stress-mediated HMGB1 release in infection, inflammation, and cell death may target the 1) CRM1-mediated nuclear export of HMGB1 in activated immune cells; 2) PARP1-medaited HMGB1 release in necrosis; 3) Caspase3/7-medaited HMGB1 release in apoptosis; 4) ATG5-medaited HMGB1 release in autophagy; 5) PKR-mediated HMGB1 release in pyroptosis; and 6) PAD4-mediated HMGB1 release in netosis. The effect of the also-based composition could also arise from the prevention of cluster formation or self-association of HMGB1 that could be achieved through targeting specific physiochemical factors such as ionic strength (increasing ionic strength reduces the strength of HMGB1 tetramer), pH (highest rate of self-association, pH 4.8), metal ions especially zinc (inclusion of low dosage Zn2+ promotes HMGB1 tetramer formation), and redox environment (in a more oxidized condition, which mimics extracellular environment, HMGB1 predominantly exists as tetramer, whereas in a more reduced condition, such as in intracellular environment, more dimer species are present). By changing the physiochemical microenvironment, the Aloe-based composition comprising polysaccharides and polyphenols prevents the formation of HMGB1 tetramer and interfere the binding affinity of HMGB1 to TLR and RAGE.

Immune function and pulmonary structure integrity and function associated “biomarkers” regulated contemplated Aloe-based compositions, including UP360, comprising and, in some embodiments, consisting of polysaccharides and polyphenols for regulation of homeostasis of immunity at various combinations of 2 to 3 of plant extracts with examples but not limited to UP360 in this disclosure, include but not limited to IL-1, IL-2, IL-4, IL-6, IL-7, IL-8, IL-10, IL-12, IL-17, GM-CSF, G-CSF, CCL2/3/5, IP-10, CXCL10, CRP, HMGB1, INF-α/β/γ, NF-κB, PDGF-BB, MIP-1α, D-dimer, angiotensin II, cardiac troponin, VEGF, PDGF, albumin, Nrf2, SOD, MDA, iNOS, COX1, COX2, LO5, LO12, LO13.

“Virus” as used herein include but not limited to highly pathogenic avian influenza (H5N1 virus strain A), influenza A (H1N1), Hepatitis virus A, B, C, and D; SARS-CoV, SARS-CoV-2 (COVID-19) MERS-CoV (MERS), Respiratory syncytial virus (RSV), Enterovirus A71 (EV71).

The amount of a compound, an extract or a composition of this disclosure that constitutes a “therapeutically effective amount” will vary depending on the bioactive compound, or the biomarker for the condition being treated and its severity, the manner of administration, the duration of treatment, or the age of the subject to be treated, but can be determined routinely by one of ordinary skill in the art having regard to his own knowledge and to this disclosure. In certain embodiments, “effective amount” or “therapeutically effective amount” may be demonstrated as the quantity of polysaccharides and polyphenols composition over the body weight of a mammal (i.e., 0.005 mg/kg, 0.01 mg/kg, or 0.1 mg/kg, or 1 mg/kg, or 5 mg/kg, or 10 mg/kg, or 20 mg/kg, or 50 mg/kg, or 100 mg/kg, or 200 mg/kg or 500 mg/kg). The human equivalent daily dosage can be extrapolated from the “effective amount” or “therapeutically effective amount” in an animal study by utilization of FDA guideline in consideration the difference of total body areas and body weights of animals and human.

“Dietary supplements” as used herein are a product that improves, promotes, increases, manages, controls, maintains, optimizes, modifies, reduces, inhibits, or prevents a homeostasis, a balance, a particular condition associated with a natural state or biological process, or a structural and functional integrity, an off-balanced or a compromised, or suppressed or overstimulated of a biological function or a phenotypic condition (i.e., are not used to diagnose, treat, mitigate, cure, or prevent disease). For example, with regard to immunity, dietary supplements may be used to modulate, maintain, manage, balance, suppress or stimulate any components of adaptive or innate immunity, as an immunoadjuvants specific to immune stimulators which enhance the efficacy of vaccine, enhance phagocytosis activity of macrophages, improve natural killing activity of NK cells, regulate level the production of proinflammatory cytokines, mitigate inflammation and tissue damage, induce response and production of antibodies, enhance antibody dependent cellular cytotoxicity, stimulate T-cell proliferation, promote the generation of immunosuppressive regulatory T-cells, and protect immune and lung cells from HMGB1 induced cytokine storm damage, and protect organs and/or tissues from oxidative stress. In certain embodiments, dietary supplements are a special category of food, functional food, medical food, nutrient, nutritional product, and are not a drug.

“Treating” or “treatment” as used herein refers to the treatment of the disease or condition of interest in a mammal, such as a human, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving or modifying the disease or condition, i.e., causing regression of the disease or condition; or (iv) relieving the symptoms resulting from the disease or condition, (e.g., relieving cough and fever, relieving pain, reducing inflammation, reducing lung edema, mitigating pneumonia) without addressing the underlying disease or condition; (v) balancing the regulation of immunity homeostasis or changing the phenotype of the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians. A disease or condition may be acute such as virus infection (SARS, COVID-19, MERS, Hepatitis, influenza) or microbial infection; and may be chronic such as lung damage caused by exposure to air pollution, and to smoke. A compromised immune function from off balance of homeostasis could cause a disease or a condition, or could make the Mammal more susceptible infectious diseases, mutation of cells, or could lead to more secondary organ and tissue damages directly or indirectly associated with infections from virus or microbials or air pollutants.

As used herein, “statistical significance” refers to a p value of 0.05 or less when calculated using the Students t-test and indicates that it is unlikely that a particular event or result being measured has arisen by chance.

For the purposes of administration, the compounds of the present contemplated subject matter may be administered as a raw ingredient or may be formulated as pharmaceutical or nutraceutical compositions. Pharmaceutical or nutraceutical compositions of the present contemplated subject matter comprise a compound of structures described in this contemplated subject matter and a pharmaceutically or nutraceutically acceptable carrier, diluent or excipient. The compound of structures described here are present in the composition in an amount which is effective to treat a particular disease or condition of interest—that is, in an amount sufficient to promote innate or adaptive immunity or immunity homeostasis in general or any of the other associated indications described herein, and generally with acceptable toxicity and/or safety profile to a patient.

Administration of the compounds or compositions of this disclosure, or their pharmaceutically or nutraceutically acceptable salts, in pure form or in an appropriate pharmaceutical or nutraceutical composition, can be carried out via any of the accepted modes of administration of agents for serving similar utilities. The pharmaceutical or nutraceutical compositions of this disclosure can be prepared by combining a compound of this disclosure with an appropriate pharmaceutically or nutraceutically acceptable carrier, diluent or excipient, and may be formulated into preparations in solid, semi solid, liquid or gaseous forms, such as tablets, capsules, powders, granules, soft gel, gummy, ointments, solutions, beverage, suppositories, injections, inhalants, gels, creams, lotions, tinctures, sashay, ready to drink, masks, microspheres, and aerosols. Typical routes of administering such pharmaceutical or nutraceutical compositions include oral, topical, transdermal, inhalation, parenteral, sublingual, buccal, rectal, vaginal, or intranasal. The term parenteral as used herein includes subcutaneous injections, intravenous, intramuscular, intrasternal injection or infusion techniques. In contemplated embodiments, administering the composition is selected from the group comprising oral administration, topical administration, suppository administration, intravenous administration, intradermic administration, intragastric administration, intramuscular administration, intraperitoneal administration, and intravenous administration.

Pharmaceutical or nutraceutical compositions of this disclosure are formulated so as to allow the active ingredients contained therein to be bioavailable upon administration of the composition to a patient. Compositions that will be administered to a subject or patient or a mammal take the form of one or more dosage units, where for example, a tablet may be a single dosage unit, and a container of a compound or an extract or a composition of 2-3 plant extracts of this disclosure in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of this disclosure, or a pharmaceutically or nutraceutically acceptable salt thereof, for treatment of a disease or condition of interest in accordance with the teachings of this contemplated subject matter.

The composition of pharmaceutical or nutraceutical compositions of this disclosure, wherein the active, adjuvant, excipient or carrier is selected from one or more of Cannabis sativa oil or extract, or CBD or THC, turmeric extract or curcumin, terminalia extract, willow bark extract, Devil's claw root extract, cayenne pepper extract or capsaicin, Prickly Ash bark extract, philodendra bark extract, hop extract, Boswellia extract, rose hips extract, green tea extract, Sophora extract, Mentha or Peppermint extract, ginger or black ginger extract, green tea or grape seed polyphenols, Omega-3 and/or Omega-6 Fatty Acids, Krill oil, gamma-linolenic acid, citrus bioflavonoids, Acerola concentrate, astaxanthin, American ginseng, Asia ginseng, elderberry, garlic extract, garlic oil, echinacea extract, agave nectar, eucalyptus oil, ascorbic acid, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, beta-cyclodextrin, cellulose, dextrose, saline, water, oil, shark and bovine cartilage.

A pharmaceutical or nutraceutical composition of this disclosure may be in the form of a solid or liquid. In one aspect, the carrier(s) are particulate, so that the compositions are, for example, in tablet or in powder form. The carrier(s) may be liquid, with the compositions being, for example, oral syrup, injectable liquid or an aerosol, which is useful in, for example, inhalatory administration.

When intended for oral administration, the pharmaceutical or nutraceutical composition is in either solid or liquid form, where semi solid, semi liquid, suspension and gel forms are included within the forms considered herein as either solid or liquid.

As a solid composition for oral administration, the pharmaceutical or nutraceutical composition may be formulated into a powder, granule, compressed tablet, pill, capsule, chewing gum, gummy, soft gel, sashay, wafer, bar, or like form. Such a solid composition will typically contain one or more inert diluents or edible carriers. In addition, one or more of the following may be present: binders such as carboxymethylcellulose, ethyl cellulose, cyclodextrin, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical or nutraceutical composition is in the form of a capsule, for example, a gelatin capsule, it may contain, in addition to materials of the above type, a liquid carrier such as polyethylene glycol or oil.

The pharmaceutical or nutraceutical composition may be in the form of a liquid, for example, an elixir, tincture, syrup, solution, emulsion or suspension. The liquid may be for oral administration or for delivery by injection, as two examples. When intended for oral administration, a useful composition contains, in addition to the present compounds, one or more of a sweetening agent, preservatives, emulsifier, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical or nutraceutical compositions of this disclosure, whether they be solutions, suspensions or other like form, may include one or more of the following adjuvants: sterile diluents such as water for injection, saline solution, such as physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a generally useful adjuvant. An injectable pharmaceutical or nutraceutical composition is sterile.

A liquid pharmaceutical or nutraceutical composition of this disclosure intended for either parenteral or oral administration should contain an amount of a compound of this disclosure such that a suitable dosage will be obtained.

The pharmaceutical or nutraceutical composition of this disclosure may be intended for topical administration, in which case the carrier may suitably comprise a solution, emulsion, cream, lotion, ointment, or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical or nutraceutical composition for topical administration. If intended for transdermal administration, the composition may include a transdermal patch or iontophoresis device.

The pharmaceutical or nutraceutical composition of this disclosure may be intended for rectal administration, in the form, for example, of a suppository, which will melt in the rectum and release the drug. The composition for rectal administration may contain an oleaginous base as a suitable nonirritating excipient. Such bases include lanolin, cocoa butter and polyethylene glycol.

The pharmaceutical or nutraceutical composition of this disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical or nutraceutical composition of this disclosure in solid or liquid form may include an agent that binds to the compound of this disclosure and thereby assists in the delivery of the compound. Suitable agents that may act in this capacity include a monoclonal or polyclonal antibody, a protein or a liposome.

The pharmaceutical or nutraceutical composition of this disclosure in solid or liquid form may include reducing the size of a particle to, for example, improve bioavailability. The size of a powder, granule, particle, microsphere, or the like in a composition, with or without an excipient, can be macro (e.g., visible to the eye or at least 100 μm in size), micro (e.g., may range from about 100 μm to about 100 nm in size), nano (e.g., may no more than 100 nm in size), and any size in between or any combination thereof to improve size and bulk density.

The pharmaceutical or nutraceutical composition of this disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of this disclosure may be delivered in single phase, bi phasic, or tri phasic systems in order to deliver the active ingredient(s). Delivery of the aerosol includes the necessary container, activators, valves, sub-containers, and the like, which together may form a kit. One skilled in the art, without undue experimentation, may determine the most appropriate aerosol(s).

The pharmaceutical or nutraceutical compositions of this disclosure may be prepared by methodology well known in the pharmaceutical or nutraceutical art. For example, a pharmaceutical or nutraceutical composition intended to be administered by injection can be prepared by combining a compound of this disclosure with sterile, distilled, deionized water so as to form a solution. A surfactant may be added to facilitate the formation of a homogeneous solution or suspension. Surfactants are compounds that non covalently interact with the compound of this disclosure so as to facilitate dissolution or homogeneous suspension of the compound in the aqueous delivery system.

The compounds of this disclosure, or their pharmaceutically or nutraceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of this disclosure, or pharmaceutically or nutraceutically acceptable derivatives thereof, may also be administered simultaneously with, prior to, or after administration of food, water and one or more other therapeutic agents. Such combination therapy includes administration of a single pharmaceutical or nutraceutical dosage formulation which contains a compound or an extract or a composition with 2-3 plant extracts of this disclosure and one or more additional active agents, as well as administration of the compound or an extract or a composition with 2-3 plant extracts of this disclosure and each active agent in its own separate pharmaceutical or nutraceutical dosage formulation. For example, a compound or an extract or a composition with 2-3 plant extracts of this disclosure and another active agent can be administered to the patient together in a single oral dosage composition, such as a tablet or capsule, or each agent can be administered in separate oral dosage formulations. Where separate dosage formulations are used, the compounds of this disclosure and one or more additional active agents can be administered at essentially the same time, i.e., concurrently, or at separately staggered times, i.e., sequentially; combination therapy is understood to include all these regimens.

It is understood that in the present description, combinations of sub stituents or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include C(O)R″ (where R″ is alkyl, aryl or arylalkyl), p methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this contemplated subject matter may not possess pharmacological activity as such, they may be administered to a mammal and thereafter metabolized in the body to form compounds of this disclosure which are pharmacologically active. Such derivatives may therefore be described as “prodrugs”. All prodrugs of compounds of this contemplated subject matter are included within the scope of this disclosure.

Furthermore, all compounds or extracts of this disclosure which exist in free base or acid form can be converted to their pharmaceutically or nutraceutically acceptable salts by treatment with the appropriate inorganic or organic base or acid by methods known to one skilled in the art.

Salts of the compounds of this disclosure can be converted to their free base or acid form by standard techniques.

Contemplated compounds, medicinal compositions and compositions may comprise or additionally comprise or consist of at least one active ingredient. In some embodiments, at least one bioactive ingredient may comprise or consist of plant powder or plant extract of or the like.

In any of the aforementioned embodiments, the compositions comprising mixtures of extracts or compounds may be mixed at a particular ratio by weight. For example, an aloe leaf gel powder containing polysaccharides and a rosemary extract containing Rosmarinic acid may be blended in a 1:2 weight ratio, respectively. In certain embodiments, the ratio (by weight) of two extracts or compounds of this disclosure ranges from about 0.5:5 to about 5:0.5. Similar ranges apply when more than two extracts or compounds (e.g., three, four, five) are used. Exemplary ratios demonstrated in example 10 include 0.5:1, 0.5:2, 0.5:3, 0.5:4, 0.5:5, 1:1, 1:2, 1:3, 1:4, 1:5, 2:1, 2:2, 2:3, 2:4, 2:5, 3:1, 3:2, 3:3, 3:4, 3:5, 4:1, 4:2, 4:3, 4:4, 4:5, 5:1, 5:2, 5:3, 5:4, 5:5, 1:0.5, 2:0.5, 3:0.5, 4:0.5, or 5:0.5. In certain embodiments, the disclosed individual extracts of Aloe, and/or Poria, and/or Rosemary are blended into a composition with 3 individual extracts demonstrated in example 9 and 10 in a 1:1:1, 2:1:1, 3:1:1, 4:1:1, 5:1:1, 1:2:1, 1:3:1, 1:4:1, 1:5:1, 1:1:2, 1:1:3, 1:1:4, 1:1:5, 1:2:3, 1:2:4, 1:2:5, 1:2:6, 1:2:6, 1:2:8, 1:2:9 or 1:2:10 etc weight ratio, respectively. In further embodiments, the disclosed individual extracts of Aloe, Poria, and Rosemary have been combined into a contemplated composition called UP360 as an example but not limited to a blending ratio of 3:6:1 or 1:1:1 or 3:2:1 as of Aloe:Poria:Rosemary.

In further embodiments, such combinations of individual extracts of aloe, poria, and rosemary at various combinations of 2 to 3 of those extracts with examples but not limited to UP360 comprising polysaccharides and polyphenols, were evaluated on in vitro, and/or ex vivo and/or in vivo models for advantage/disadvantage and unexpected synergy/antagonism of the perceived biological functions and effective adjustments of the homeostasis of immune function and mitigate the organ damages caused by cytokine storm, oxidative stress, and sepsis. The best compositions with specific blending ratio of individual extracts of Aloe, or Poria, or Chaga or Rosemary were selected based on unexpected synergy measured on the in vitro, and/or ex vivo and/or in vivo models due to the diversity of chemical components in each extract and different mechanism of actions from different types of bioactive compounds in each extract, and potential enhancement of ADME of natural compounds in the composition to maximize the biological outputs.

In any of the aforementioned embodiments, the compositions comprising mixtures of extracts or compounds may be present at certain percentage levels or ratios. In certain embodiments, a composition comprising an aloe whole leaf or inner leaf gel powder (Example 3 & 4) and/or a Rosemary extract (Example 6) can include 0.1% to 49.9% or about 2% to about 40% or about 0.5% to about 10% of polysaccharides, 0.1% to 99.9% or about 1% to about 10% or about 5% to about 50% of rosmarinic acid, or a combination thereof. In certain embodiments, a composition comprising an Aloe vera gel powder (Example 3 & 4), or Poria aqueous extract (Example 5) or rosemary extract (Example 6) can include from about 0.01% to about 99.9% polysaccharides or include at least 1%, 2%, 3%, 4%, 5%, 6%, 7%, 8%, 9%, 10%, 11%, 12%, 13%, 14%, 15%, 16%, 17%, 18%, 19%, 20%, 30%, 35%, 40%, 45%, 50%, 55%, 60%, 65%, 70%, 75%, 80% or 90% rosmarinic acid.

In certain examples (Example 9), a composition of this disclosure may be formulated to further comprise a pharmaceutically or nutraceutically acceptable carrier, diluent, or excipient, wherein the pharmaceutical or nutraceutical formulation comprises from about 0.05 weight percent (wt%), or 0.5 weight percent (wt%), or 5%, or 25% to about 95 wt% of active or major active ingredients of an extract mixture. In further embodiments (Example 9), the pharmaceutical or nutraceutical formulation comprises from about 0.05 weight percent (wt%) to about 90 wt% polysaccharides, about 0.5 wt% to about 80 wt% rosmarinic acid, about 0.5wt% to about 75 wt% total polyphenols, about 0.5 wt% to about 70 wt%, about 0.5 wt% to about 50 wt%, about 1.0 wt% to about 40 wt%, about 1.0 wt% to about 20 wt%, about 1.0 wt% to about 10 wt%, about 3.0 wt% to about 9.0 wt%, about 5.0 wt% to about 10 wt%, about 3.0 wt% to about 6wt% of the major active ingredients in an extract mixture, or the like. In any of the aforementioned formulations, a composition of this disclosure is formulated as a tablet, hard capsule, soft gel capsule, powder, or granule.

Also contemplated herein are agents of the disclosed compounds. Such products may result from, for example, the oxidation, reduction, hydrolysis, amidation, esterification, and the like of the administered compound, primarily due to enzymatic processes. Accordingly, contemplated compounds are those produced by a process comprising administering a contemplated compound or composition to a mammal for a period of time sufficient to yield a metabolic product thereof. Such products are typically identified by administering a radiolabeled or not radiolabeled compound of this disclosure in a detectable dose to an animal, such as rat, mouse, guinea pig, dog, cat, pig, sheep, horse, monkey, or human, allowing sufficient time for metabolism to occur, and then isolating its conversion products from the urine, blood or other biological samples.

Contemplated compounds, medicinal compositions and compositions may comprise or additionally comprise or consist of at least one pharmaceutically or nutraceutically or cosmetically acceptable carrier, diluent or excipient. As used herein, the phrase “pharmaceutically or nutraceutically or cosmetically acceptable carrier, diluent or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. Contemplated compounds, medicinal compositions and compositions may comprise or additionally comprise or consist of at least one pharmaceutically or nutraceutically or cosmetically acceptable salt. As used herein, the phrase “pharmaceutically or nutraceutically or cosmetically acceptable salt” includes both acid addition and base addition salts.

Natural immune suppressants are the molecules that inhibit the immune system, suppress chronic systematic inflammation and oxidative stress, protect immune and lung cells from HMGB1 induced cytokine storm damage, provide potent antioxidant to reduce oxidative stress and decrease NF-kb, and reduce proinflammatory pathways (COX/LOX and cytokines—IL-1, IL-6, TNF-a), therefore can be used to control the physiological and/or pathological immune reactions in order to achieve homeostasis of the immune function as demonstrated in the current contemplated subject matter. Those phenolic natural compounds illustrated in above include but not limited to Rosmarinic acid, Kaempferol, genistein, quercetin, Butein, Luteolin, chrysin, Apigenin, curcumin, resveratrol, capsaicin, glomeratose A, 6-shogaol, gingerol, Zingerone, berberine, piperine, Epigallocatechin, Colchicine, Lycorine,

It is contemplated that rosmarinic acid is derived, obtained or selected from at least one of the following—alone or in combination with one of all plant parts of Rosmarinus officinalis, Melissa officinalis, Momordica balsamina, Mentha piperita, Perilla frutescens, Salvia officinalis, Teucrium scorodonia, Sanicula europaea, Coleus blumei, Thymus spp., Hyptis verticillata, Lithospermum erythrorhizon and hornwort Anthoceros agrestis or a combination thereof.

The plant species that contain above immune suppressing natural phenolic compounds including but not limited to Piper longum Linn, Coptis chinensis Franch, Angelica sinensis (Oliv.) Diels, Sophora flavescens Ait, Toxicodendron vernicifluum, Glycyrrhiza glabra, Curcuma longa, Salvia Rosmarinus, Rosmarinus officinalis, Zingiber officinalis, Polygala tenuifolia, Humulus lupulus, Lonicera Japonica, Salvia officinalis L., Centella asiatica, Boswellia carteri, Mentha longifolia, Picea crassifolia, Citrus nobilis Lour, Citrus aurantium L. Camellia sinensis L. Pueraria mirifica, Pueraria lobata, Glycine max, Lycoris radiate, Colchicum autumnale, Capsicum species, Fallopia japonica, Many phenolic compounds can also be found in various fruits and vegetables e.g., tea, tomato, cruciferous vegetables, grapes, blueberries, elderberries, raspberries, cranberries, mulberries, apple, chili peppers etc.

Natural polysaccharides that stimulate innate immunity, enhance adaptive immunity especially CD3+, CD4+, CD8+, NKp46+ Natural Killer cells, TCRγδ+ Gamma delta T-cells, and CD4+TCRγδ+ Helper Gamma delta T-cells, protect immune and lung cells from HMGB1 induced cytokine storm damage, maintain homeostasis of innate and adaptive immune responses by formulating with a immune suppressing phenolic natural compounds including but not limited with Mannan, acetyl mannan, Galactomannans, glucans, beta-glucans (from the poria extract for example), α-1,6 and α-1,4 glucans, β-1,3-glucans, fucoidans, frucans, and pectins.

The plant species that contain above immune stimulating natural polysaccharide compounds including but not limited to Aloe vera, Aloe barbadense, Aloe ferox, Aloe arborescens, Astragalus membranaceus, Ganoderma lucidum, Hordeum vulgare, Agaricus (A. blazei) subrufescens, Echinacea purpurea, Echinacea angustifolia, Aconitum Napellus (Monkshood), Sambucus nigra, Poria cocos Wolf, Wolfiporia extensa, Withania somnifera, Bupleurum falcatum, Radix Bupleuri, Radix Glycyrrhiza, Fructus Forsythiae, Panax quinquefolium, Panax ginseng C. A. Meyer, Korea red ginseng, Lentinula edodes (shiitake), Inonotus obliquus (Chaga mushroom), Lentinula edodes, Lycium barbarum, Phellinus linteus (fruit body), Trametes versicolor (fruit body), Cyamopsis tetragonolobus Cyamopsis tetragonolobus (guar gum), Trametes versicolor, Cladosiphon okamuranus Tokida, Undaria pinnatifida. Many polysaccharide compounds can also be found in various fruits and vegetables e.g., mushrooms, seaweeds, yeasts, brown algae, Agave Nectar, brown seaweed, fermentable fiber, cereal, sea cucumber, agave, artichokes, asparagus, leeks, garlic, onions, rye, barley kernels, wheat, pears, apples, guavas, quince, plums, gooseberries, oranges and other citrus fruits.

Acetylated polysaccharides are part of plant cell wall polymers. Acetylated polysaccharides were reported with higher antioxidant, better immune modulation properties compared to regular polysaccharides. The degree of O-acetylation can vary depending on the species, parts, and developmental state. Acetyl content in some natural polysaccharides has been demonstrated to play an important role in their bioactivities, although o-acetylation mechanism is still not fully understood.

In some embodiments, polysaccharides and/or phenolic compounds or extracts of the present disclosure can be isolated from plant and/or marine sources, for example, from those plants included in the Examples 1-8 and elsewhere throughout the present application. Suitable plant parts for isolation of the compounds include leaves, bark, trunk, trunk bark, stem, stem bark, twigs, tubers, root, rhizome, root bark, bark surface, young shoots, seed, fruit, fruit body, androecium, gynoecium, calyx, stamen, petal, sepal, carpel (pistil), flower, or any combination thereof. In some related embodiments, the compounds or extracts are isolated from plant sources, total synthesized, biosynthesized with plant or fungi tissues, stem cells and transgenic microbials and synthetically modified to contain any of the recited substituents. In this regard, synthetic modification of the compound isolated from plants can be accomplished using any number of techniques which are known in the art and are well within the knowledge of one of ordinary skill in the art.

Other embodiments of the contemplated subject matter relate to methods of use of the Aloe-based compositions comprising polysaccharides and polyphenols for regulation of homeostasis of immunity at various combinations of 2 to 3 of plant extracts with examples but not limited to UP360 in this disclosure, include but not limited to optimizing and/or balancing the immune response; helping to maintain a healthy immune function against virus infection and bacterial infections; protecting immune system from oxidative stress damage induced by air pollution and smoking; protecting normal healthy lung function from virus infection, bacterial infections and air pollution; supporting healthy inflammatory response; maintaining healthy level of cytokines and cytokine responses to infections; elevating and maintaining anti-inflammatory cytokines such as IL-10; controlling oxidative response and alleviating oxidative stress; maintaining lung cleanse and detox capability; protecting lung structure integrity and oxygen exchanging capacity; maintaining respiratory passages and enhancing oxygen absorption capacity of alveoli; mitigating oxidative stress caused pulmonary damage; promoting microcirculation of the lung and protecting normal coagulation function; increasing the activity and count of the white blood cells, enhancing Natural Killer (NK) cell function; increasing the count of T and B lymphocytes; increasing CD3+, CD4+ NKp46+ Natural Killer cells, TCRγδ+ Gamma delta T-cells, and CD4+TCRγδ+ Helper Gamma delta T cells and CD8+ cell counts; protecting and promoting macrophage phagocytic activity; supporting and/or promoting normal antibody production; maintaining healthy pulmonary microbiota and/or symbiotic system in respiratory organs; relieving and/or reducing cold/flu-like symptoms including but not limited to body aches, sore throat, cough, minor throat and bronchial irritation, nasal congestion, sinus congestion, sinus pressure, runny nose, sneezing, loss of smell, loss of taste, muscle sore, headache, fever and chills; helping loosen phlegm (mucus) and thin bronchial secretions to make coughs more productive; reducing severity of bronchial irritation;

reducing severity of lung damage and/or edema and/or inflammatory cell infiltration caused by virus infection, microbial infection and air pollution; supporting bronchial system and comfortable breathing through the cold/flu and/or pollution seasons; preventing and/or treating lung fibrosis; reducing duration and/or severity of common cold/flu; reducing severity and/or duration of virus and bacterial infection of respiratory system; preventing, and/or treating and/or curing respiratory infections caused by virus, microbial, and air pollutants; managing and/or treating and/or preventing, and/or reversing the progression of respiratory infections; promoting and strengthening and rejuvenating the repair and renewal function of lung and the entire respiratory system or the like.

### EXAMPLES

### Example 1

### Preparation of Organic and Aqueous Extracts

Dried ground plant powder of each plant (20 g) was loaded into 100 ml stainless steel tube and extracted twice with an organic solvent mixture (methylene chloride/methanol in a ratio of 1:1) using an ASE 300 automatic extractor at 80° C. and 1500 psi. The extract solution was automatically filtered and collected, then followed by flushing with fresh solvent and purging with nitrogen gas to dryness before switching to aqueous extraction at 50° C. The combined organic extract solution was evaporated with a rotary evaporator to give crude organic extract (OE). The biomass was air dried and extracted once with DI water. The aqueous solution was filtered and freeze-dried to provide aqueous extract (AE).

Similar results were obtained using the same procedure, but with the organic solvent mixture being replaced with methanol or ethanol to provide a methanol extract (ME) or ethanol extract (EE), Ethanol:H₂O (7:3) extracts, Ethanol:H₂O (1:1) extracts, Ethanol:H₂O (3:7) extracts and water extracts respectively.

### Example 2

### Sample Preparation of Aloe vera Leaf Gel Powder

The fresh leaves of Aloe vera plant were washed, and the outer rinds were removed. The exudates of leaf gel were treated with cellulose enzyme and filtered through activated charcoal. The filtrates were concentrated by low pressure evaporation and dehydrated to dry power by freeze-drying, spread drying or Qmatrix® processing. Aloe vera leaf gel powder was produced in the form of the lyophilizate at a ratio of 200:1 to 100:1 with no less than 8% polysaccharides.

### Example 3

### Preparation of Aloe Polysaccharides by Ethanol Precipitation Method

The Aloe leaf gel powder was dissolved in water at a concentration of 40 g/L, and ethyl alcohol was added to the solution slowly during constant stirring with a magnetic stir bar to bring the solution up to 80% ethanol. The precipitates were separated from the supernatants by centrifuge at 2500 rpm and dried down by Speedvac. In total, 1 kg of Aloe leaf gel powder (Lot WM 180141) could be processed to give 379 g precipitate. The precipitate and supernatant were submitted for Size Exclusion Column (SEC) chromatography with HPLC analysis, which revealed that no polysaccharides above 10K were detected in the supernatant and the precipitate contained 40.5% polysaccharides with molecular weight larger than 10 KD (Table 1).

TABLE 1
The contents of polysaccharides in aloe precipitate and supernatants

| | Polysaccharides molecular weight distribution (KD) | | | | | | |
|---|---|---|---|---|---|---|---|
| Samples | 10-50 | 50-200 | 200-500 | 500-1,000 | 1,000-2,000 | >2,000 | >10 |
| Precipitate | 22.26% | 33.45% | 17.78% | 10.02% | 7.51% | 8.99% | 40.5% |
| Supernatant | nd | nd | nd | nd | nd | nd | nd |

nd = not detected

### Example 4

### Preparation of Three Aloe Polysaccharides Fractions by Ultrafiltration

379 g aloe precipitate was dissolved in water at a concentration of 20 g/L. The water solution was subjected to ultrafiltration (BONA-GM-18 from Jinan Bona Biotechnology) at a flow rate of 0.5-10 L per hour, passing through organic ultrafiltration membranes sequentially with different pore sizes to filter out polysaccharides with molecular weights 1 KD, 5 KD, 50 KD, 300 KD and 500 KD, respectively. Three polysaccharides fractions: >500 kD (45.7 g), 50-500 kD (30.1 g) and 5-50 KD (19.8 g) were collected and dried down with freeze dry lyophilizer. The molecular weight distributions and polysaccharide purities were analyzed with SEC HPLC & NMR methods.

### Example 5

### Preparation of Poria cocos Extracts

Dried ground plant Poria cocos sclerotium powder (20 g) was loaded into 100 ml stainless steel tube and extracted twice with an organic solvent mixture (methylene chloride/methanol in a ratio of 1:1 using an ASE 300 automatic extractor at 80° C. and 1500 psi. The extract solution was automatically filtered and collected, then followed by flushing with fresh solvent and purging with nitrogen gas to dryness before switching to aqueous extraction at 50° C. The combined organic extract solution was evaporated with rotary evaporator to give crude organic extract (OE) 0.82 g (4.10% yield). The biomass was air-dried and extracted once with water. The aqueous solution was filtered and freeze-dried to provide aqueous extract (AE) 0.51 g (2.55% yield).

Dried ground plant Poria cocos sclerotium powder (20 g) was loaded into 100 ml stainless steel tube and extracted twice with ethanol using an ASE 300 automatic extractor at 80° C. and 1500 psi. The extract solution was automatically filtered and collected, then followed by flushing with fresh solvent and purging with nitrogen gas to dryness before switching to aqueous extraction at 50° C. The combined organic extract solution was evaporated with rotary evaporator to give crude ethanol extract 0.3893 g (1.95% yield). The biomass was air dried and extracted once with water. The aqueous solution was filtered and freeze-dried to provide aqueous extract (AE) 0.3581 g (1.79% yield).

Similar results were obtained using the same procedure, but with the organic solvent being replaced with methanol or ethanol to provide a methanol extract (ME) or ethanol extract (EE), Ethanol:H₂O (7:3) extracts, Ethanol:H₂O (1:1) extracts, Ethanol:H₂O (3:7) extracts and water extracts respectively.

The dried ground fruiting body powder of Poria cocos was extracted by water to give Poria water extract with Lot# 210317 at an extraction yield of 15 to 1. The polysaccharides in Poria extracts were determined by colorimetric method using the Phenol—Sulfuric Acid method with a UV wavelength of 490 nm against glucose. Total triterpenoids in Poria extract was quantified by Vanillin-Sulphuric Acid Method at a UV wavelength of 548 nm against Oleanolic acid. The active content for different Poria extracts with polysaccharides content in a range of 10-40% by colorimetric method (Table 2).

TABLE 2
The active contents of Poria cocos extracts

| | | Active contents |
|---|---|---|
| Material | Sample ID | Quantified by UV Methods |
| Poria coccos extract | L0761 | 20% Polysaccharides |
| Poria coccos extract | L0770 | 21.73% Polysaccharides |
| | | 3.56% Triterpenes |
| Poria coccos extract | L501 | Polysaccharides 7.22% |
| | | Triterpenes 16.2% |
| Poria cocos extract | L0784 | 39.72% Polysaccharide |
| | | 4.32% Triterpenes |
| Poria cocos extract | L0501-2 | 20% Polysaccharide |
| | | 10% Triterpenes |
| Poria cocos extract | L696 | 30% Polysaccharides |

TABLE 3
Molecular weight distribution of Poria
polysaccharides based on SEC HPLC analysis

| Poria Extract Sample ID | L784 | L770 | P00482 | 201109-2 |
|---|---|---|---|---|
| Polysaccharides (>5 kD) | 33.05% | 38.21% | 8.71% | 7.67% |
| >2000K | 0% | 0% | 0% | 0.00% |
| 2000K-1000K | 0.03% | 0% | 0% | 0.00% |
| 1000K-500K | 0.29% | 0% | 0% | 0.00% |
| 500K-200K | 0.95% | 0.11% | 0% | 0.10% |
| 200K-50K | 5.02% | 2.23% | 0% | 1.18% |
| 50K-5K | 69.77% | 75.17% | 11.91% | 15.63% |
| 5K-0.5K | 23.93% | 22.48% | 88.00% | 83.09% |

Poria extract samples with polysaccharides were prepared at 20 mg/mL concentration and analyzed by size-exclusion chromatography (SEC) HPLC with a PolySep-SEC-P5000 column (Phenomenex OOH-3145KO, 30×0.78 cm,) at 50° C. with an isocratic elution of 100 mM NaCl solution at a flow rate of 0.7 mg/min detected by a RI detector using a series of Dextran molecular weight standards from a molecular weight range of 9.9 KDa to 2,285 KDa. The polysaccharides are integrated with vertical cursor on the target peak based on each molecular weight cutoff (pre-calculated from standard calibration as appropriate). The polysaccharides distribution and total polysaccharides content were calculated for each sample as shown in Table 3.

The total polysaccharides content with molecular weight above 5 KDa was calculated as 33.05% in Poria extract L784 by this SEC -HPLC method, mainly in the range of 5-2000 KDa. Poria polysaccharides content varies in a range between 5%-40% by this SEC-HPLC method.

### Example 6

### Preparation of Rosemary Extracts

Dried ground plant Rosmarinus officinalis aerial parts powder (20 g) was loaded into 100 ml stainless steel tube and extracted twice with an organic solvent mixture (methylene chloride/methanol in a ratio of 1:1 using an ASE 300 automatic extractor at 80° C. and 1500 psi. The extract solution was automatically filtered and collected, then followed by flushing with fresh solvent and purging with nitrogen gas to dryness before switching to aqueous extraction at 50° C. The combined organic extract solution was evaporated with rotary evaporator to give crude organic extract (OE) 2.19 g (10.95% yield). the biomass was air dried and extracted once with water. The aqueous solution was filtered and freeze-dried to provide aqueous extract (AE) 1.26 g (6.31% yield).

Dried Rosemary leaves were extracted with ethanol/water and the filtrate was concentrated. The upper liquid was separated and further dried by vacuum and enriched by column to give rosemary extract with Rosmarinic acid content in a range of 5-95%.

Similar results were obtained using the same procedure, but with the organic solvent mixture being replaced with methanol or ethanol to provide a methanol extract (ME) or ethanol extract (EE), Ethanol:H₂O (7:3) extracts, Ethanol:H₂O (1:1) extracts, Ethanol:H₂O (3:7) extracts and water extracts respectively.

Dried Rosemary leaves were extracted by mixed solvent of ethanol and water and further extracted by ethyl acetate to give Rosmarinic acid-enriched rosemary extract with about 30% rosmarinic acid at a extraction ratio of 100:1. Rosmarinic acid extract was detected and quantified by HPLC with a content in a range of 10-90% (Table 4).

TABLE 4
The active content of Rosemary extracts

| | | Active contents |
|---|---|---|
| Material | Sample ID | Quantified with HPLC |
| Rosemary Leaf Extract | L0753 | 30.14% Rosemarinic acid |
| Rosemary Leaf Extract | L0780 | 30.11% Rosemarinic acid |
| Rosemary Leaf Extract | L0781 | 30.6% Rosemarinic acid |
| Rosemary Extract | L0752 | Rosmarinic acid 30% |
| Organic Rosemary Extract | L0785 | Rosmarinic acid 30% |
| Rosemary Leaf Extract | L752-1 | 10% Rosmarinic Acid |
| Rosemary Leaf Extract | L752-2 | 25% Rosmarinic Acid |
| Rosemary Leaf Extract | L752-3 | 40% Rosmarinic Acid |
| Rosemary Leaf Extract | L752-4 | 40% Rosmarinic Acid |
| Rosemary Leaf Extract | L752-5 | 90% Rosmarinic Acid |

### Example 7

### Preparation of Chaga Mushroom Extracts

Dried ground plant Chaga mushroom (Inonotus obliquus) powder (20 g) was loaded into 100 ml stainless steel tube and extracted twice with an organic solvent mixture (methylene chloride/methanol in a ratio of 1:1) using an ASE 300 automatic extractor at 80° C. and =1500 psi.

The extract solution was automatically filtered and collected, then followed by flushing with fresh solvent and purging with nitrogen gas to dryness before switching to aqueous extraction at 50° C. The combined organic extract solution is evaporated with rotary evaporator to give crude organic extract (OE). The biomass was air-dried and extracted once with water. The aqueous solution was filtered and freeze-dried to provide aqueous extract (AE).

Ground, dried Chaga mushroom (Inonotus obliquus) powder was extracted with water to give the water extract at a ratio of 4:1 with polysaccharides content in a range of 5-95%. Similar results were obtained using the same procedure, but with the organic solvent mixture being replaced with methanol or ethanol to provide a methanol extract (ME) or ethanol extract (EE), Ethanol:H₂O (7:3) extracts, Ethanol:H₂O (1:1) extracts, Ethanol:H₂O (3:7) extracts and water extracts respectively.

### Example 8

### Preparation of Astragalus membranaceus Extracts

Dried ground plant Astragalus membranaceus root powder (20 g) was loaded into 100 ml stainless steel tube and extracted twice with an organic solvent mixture (methylene chloride/methanol in a ratio of 1:1 using an ASE 300 automatic extractor at 80° C. and 1500 psi. The extract solution was automatically filtered and collected, then followed by flushing with fresh solvent and purging with nitrogen gas to dryness before switching to aqueous extraction at 50° C. The combined organic extract solution was evaporated with rotary evaporator to give crude organic extract (OE) 1.68 g (8.42% yield). The biomass was air-dried and extracted once with water. The aqueous solution was filtered and freeze-dried to provide aqueous extract (AE) 2.93 g (14.68% yield).

Ground, dried Astragalus membranaceus root powder was extracted with water at a ratio of 1:8 twice to give the water extract at an extraction yield of 4 to 1 with no less than 10% polysaccharides. Similar results were obtained using the same procedure, but with the organic solvent mixture being replaced with methanol or ethanol to provide a methanol extract (ME) or ethanol extract (EE), Ethanol:H₂O (7:3) extracts, Ethanol:H₂O (1:1) extracts, Ethanol:H₂O (3:7) extracts and water extracts respectively.

### Example 9

### Preparation of Aloe Based Composition UP360 and Other Combinations

As demonstrated in above examples, Aloe vera leaf gel powder was produced in the form of the lyophilizate at a ratio of 200:1 with no less than 10% polysaccharides. Poria cocos extract was made by water extraction with no less than 20% polysaccharides. Rosemary leaf extracts was manufactured by ethanol/water extraction to give no less than 30% Rosmarinic acid. Three ingredients were blended at a ratio of 3:6:1 by weight to give the final combination of contemplated Aloe-based compositions, including UP360, comprising and, in some embodiments, consisting of polysaccharides and polyphenols. Two batches of UP360 with Lot #APR-05012020-1 and APR-05012020-2 (Table 5) were produced by mixing three ingredients for 1 hour with YUCHENGTECH 10L Lab dry powder mixer with polysaccharides content (>5KDa) determined at 12.07% by SEC-HPLC method as described in example 5.

Aloe vera inner gel powder, Poria cocos extract, and Rosemary leaf extracts were blended at a ratio of 1:1:1 by weight to give a combination composition UP360 with Lot #UP0319.

Aloe vera inner gel powder, Poria cocos extract, Rosemary leaf extracts and an excipient—Litesse® (Gillco) were blended at a ratio of 3:2:1:4 by weight to give another combination of composition UP360 with LOT# UP360-APR-09012020 (4.011 kg), with polysaccharides content (>5 KDa) determined at 11.01% by SEC-HPLC method (Table 6).

Aloe vera inner gel powder, Poria cocos extract, Rosemary leaf extracts and an excipient—Litesse® (Gillco) were blended at a ratio of 3:3:1:3 by weight to give another combination of composition UP360 with LOT #UP360-Lit-1. The polysaccharides content (>5 KDa) was determined at 16.73% by SEC-HPLC method (Table 6).

TABLE 5
The blending record for UP360 lot #APR-05012020-1 and APR-05012020-2.

| | | | | Theoretical | Real | |
|---|---|---|---|---|---|---|
| | Lot Number | Material Name | Specification | Qty (kg) | Qty (kg) | Batch NO. |
| 1 | L0765/L768 | Aloe vera Gel powder | 10% | 1.350 | 1.350 | APR-05012020-1 |
| | | | Polysaccharides | | | |
| 2 | L0761/L0770 | Porici cocos Extract | 20% | 2.700 | 2.702 | |
| | | | Polysaccharides | | | |
| 3 | L0753 | Rosemary Extract | 30% | 0.450 | 0.451 | |
| | | | Rosmarinic acid | | | |
| | Final composition QTY (kg) | | | | 4.494 | |
| 1 | L0765/L768 | Aloe vera Gel powder | 10% | 1.350 | 1.352 | APR-05012020-2 |
| | | | Polysaccharides | | | |
| 2 | L076/L0770 | Poria cocos Extract | 20% | 2.700 | 2.702 | |
| | | | Polysaccharides | | | |
| 3 | L0753 | Rosemary Extract | 30% | 0.450 | 0.453 | |
| | | | Rosmarinic acid | | | |

| | |
|---|---|
| Final composition QTY (kg) | 4.503 |

TABLE 6
Molecular weight distribution of polysaccharides
in UP360 determined by SEC HPLC

| Lot# | APR-05012020-1 | APR-09012020 | UP360-Lit-1 |
|---|---|---|---|
| Polysaccharides | 12.07% | 11.01% | 16.73% |
| (>5 kD) | | | |
| >2000K | 0.00% | 0.79% | 1.14% |
| 2000K-1000K | 0.00% | 1.37% | 1.33% |
| 1000K-500K | 0.00% | 1.18% | 0.93% |
| 500K-200K | 0.00% | 1.26% | 1.41% |
| 200K-50K | 0.05% | 1.53% | 2.97% |
| 50K-5K | 11.75% | 8.77% | 14.41% |
| 5K-0.5K | 88.20% | 85.49% | 77.81% |

### Example 10

### Preparation of Combination 1 & Combination 2

Combination 1 is a mixture of Aloe vera leaf gel powder (L0765, 10% polysaccharides), Poria cocos extract (L0761, 20% polysaccharides), and Rosemary extract (L0762, 30% Rosmarinic acid) at a ratio of 1:1:1 by weight of each individual ingredient.

Combination 2 is a mixture of Chaga mushroom (Inonotus obliquus) extract (L0762, 30% polysaccharides) and Astragalus membranaceus extract (L0759, Astragaloside >0.3%, Polysaccharide >10%) at a ratio of 1:1 by weight. Combination 2 could be made by mixing Chaga mushroom (Inonotus obliquus) extract and Astragalus membranaceus extract with a ratio from 1:99 to 99:1.

### Example 11

### Enzymatic Reaction of Polysaccharide-Enriched Samples with α-Amylase and polysaccharide quantification of Poria cocos Extract, Aloe vera Gel Powder and UP360 by Size Exclusive Chromatography

200 mg plant extract in 10 mL buffer solution of NaH₂PO₄.H₂O and Na₂HPO₄.7H₂O with a pH value of 6.87 was treated with 200 μL α-Amylase enzyme solution (2 mg/mL) at room temperature for overnight. The reaction mixture was dried in SpeedVac and analyzed by size exclusive chromatography.

Samples with polysaccharides were prepared at 20 mg/mL concentration and analyzed by size-exclusive chromatography HPLC with a PolySep-SEC-P5000 column (Phenomenex OOH-3145KO, 30×0.78 cm,) at 50° C. with an isocratic elution of 100 mM NaCl solution at a flow rate of 0.7 mg/min detected by a RI detector using a series of Dextran molecular weight standards from a molecular weight range of 9.9 KDa to 2,285 KDa. The polysaccharides were integrated with vertical cursor on the target peak based on each molecular weight cutoff (pre-calculated from standard calibration as appropriate). The polysaccharides distribution and total polysaccharides content were calculated for each sample.

The Poria polysaccharides (was resistant to this enzyme, with very slight changes from 33.50% to 30.04% before and after the Amylase enzyme treatment. Same for the polysaccharide in the final UP360 composition (APR-09012020) before and after the reaction. While maltodextrin, which is composed mainly of alpha-type polysaccharides, was almost completely digested by Amylase, showed only 0.97% polysaccharides (>5 Ka) after the treatment out of 64.1% before the reaction.

TABLE 7
Polysaccharides quantification with HPLC method in Poria extract,
UP360 and Maltodextrin before and after α-Amylase hydrolysis

| | Sample | | | | | |
|---|---|---|---|---|---|---|
| | Poria Extract | | UP360 | | | |
| Polysaccharide | (L0784) | | (APR-09012020) | | Maltodextrin | |
| Size | Before | After | Before | After | Before | After |
| (>5 kD) | 33.50% | 30.04% | 11.01% | 8.59% | 64.01% | 0.97% |
| >2000K | 0.86% | 0% | 0.79% | 0% | 0% | 0% |
| 2000K-1000K | 0.14% | 0.12% | 1.37% | 0% | 0% | 0% |
| 1000K-500K | 0.30% | 0.36% | 1.18% | 0.17% | 0.01% | 0% |
| 500K-200K | 0.83% | 1.00% | 1.26% | 0.34% | 0.52% | 0% |
| 200K-50K | 3.39% | 3.35% | 1.53% | 1.52% | 10.91% | 0% |
| 50K-5K | 35.68% | 34.09% | 8.77% | 10.01% | 52.88% | 1.08% |
| 5K-0.5K | 58.80% | 61.09% | 85.49% | 87.96% | 35.69% | 98.92% |

### Example 12

### Inhibition of Hyperoxia-Induced HMGB1 Release from Dysfunctional Macrophages

Macrophages cultured under hyperoxic conditions experience oxidative stress, causing them to secrete HMGB1 to the cell culture media. To determine the efficacy of the Aloe-based composition and its components in reducing the accumulation of extracellular HMGB1 of cultured macrophages, RAW 264.7 cells were exposed for 24 hours to either 21% O₂ (room air (RA)), or 95% O₂ in the presence or absence of test substance at 25 μg/mL concentration. HMGB1 levels in cell culture supernatants were determined by ELISA at a single concentration of test substance in duplicate. The data are expressed as mean±SEM of one experiment assayed in duplicate. *p<0.05, **p<0.01, ****p<0.0001 compared to macrophages treated under hyperoxia with vehicle only. Control cells were treated with Sodium Salicylate (SS) as a positive control, which attenuates hyperoxia-compromised macrophage function and oxidative-stress-induced HMGB 1 release.

TABLE 8
Anti-HMGB1 effects in RAW 264.7 cells

| Latin | | Sample | HMGB1 | P |
|---|---|---|---|---|
| name | Sample ID | description | inhibition | Value |
| Aloe vera | P00104 | Aqueous extract | 30.95 | 0.0867 |
| | aloe extract | | | |
| Aloe vera | P00104 | Inner gel 1:200 | 87.84 | <0.0001 |
| | aloe gel | | | |
| | powder | | | |
| Aloe vera | Aloesin | >95% HPLC purity | 52.47 | 0.0038 |
| Aloe vera | aloe precipitate | 35% | 91.16 | <0.0001 |
| | | polysaccharides | | |
| Poria coccos | L501 | 20% | 83.75 | <0.0001 |
| | | polysaccharides | | |
| Rosmarinus | Rosmarinic | >98% HPLC purity | 72.97 | <0.0001 |
| officinalis | acid | | | |
| Rosmarinus | P02630-AE | Aqueous extract | 75.45 | <0.0001 |
| officinalis | | | | |

### Example 13

### Hyperoxia-Induced Dysfunctional Macrophage Phagocytosis Assay

Studies have revealed that the levels of extracellular accumulation of HMGB 1 in cultured macrophages are correlated with their phagocytic ability. RAW 264.7 cells either remained at room air (21% O₂) or were exposed to 95% O₂ for 24 hours in the presence of test substance or its vehicle. Cell viability was determined by MTT assay. Each value represents the mean±SEM of 4 independent experiments, in triplicate. *, P<0.05 compared to the T24 (21% O₂; 0 μg/ml) control group.

TABLE 9
MTT assay in cultured RAW 264.7 cells

| | 12 μg/mL | | 25 μg/mL | | 50 μg/mL | | 100 μg/mL | |
|---|---|---|---|---|---|---|---|---|
| | MTT (% | P | MTT (% | P | MTT (% | P | MTT (% | P |
| Sample name | reduction) | value | reduction) | value | reduction) | value | reduction) | value |
| Rosmarinic acid | 5.63% | 0.2092 | 13.47% | 0.0036 | 20.03% | <0.0001 | 20.53% | <0.0001 |
| Chaga mushroom | 2.10% | 0.6378 | 4.97% | 0.2676 | 0.97% | 0.8283 | 5.57% | 0.2146 |
| Combination 1 | 1.03% | 0.8167 | −2.33% | 0.601 | −0.40% | 0.9285 | 6.93% | 0.1235 |
| Combination 2 | −6.20% | 0.1676 | −10.13% | 0.026 | −4.53% | 0.3111 | −3.30% | 0.46 |

TABLE 10
Phagocytosis assay in cultured RAW 264.7 cells

| | 12 μg/mL | | 25 μg/mL | | 50 μg/mL | |
|---|---|---|---|---|---|---|
| Sample name | Phagocytosis | P value | Phagocytosis | P value | Phagocytosis | P value |
| Rosmarinic acid | 51.00% | 0.0467 | 74.35% | 0.0066** | / | / |
| Chaga mushroom | / | / | 33.74% | 0.226 | 29.27% | 0.293 |
| Combination 1 | / | / | 41.42% | 0.124 | 22.31% | 0.422 |
| Combination 2 | / | / | 33.13% | 0.2343 | 99.28% | 0.0006** |

RAW 264.7 cells either remained at room air (21% O₂) or were exposed to 95% O₂ for 24 hours in the presence of test substance. Cells were then incubated with FITC-labeled latex mini-beads for 1 hour and stained with phalloidin and DAPI to visualize the actin cytoskeleton and nuclei, respectively. For quantification of phagocytic activity, at least 200 cells per group were counted and the numbers of beads per cell were represented as a percent increase compared to the 21% 02 (0 μg/ml) control group. Each value represents the mean±SEM of 3 independent experiments for each group, in duplicate. *, P<0.05 compared to the 21% O₂ (0 μg/ml) control group.

Pure Rosmarinic acid (Example 6), aqueous extract of Chaga mushroom (Example 7) and two compositions (Example 10) were tested and the results were summarized in Tables 9 and 10.

### Example 14

### UVA&UVB Induced ROS Assay in HaCaT Cell

HaCaT cells (Human immortal keratinocyte cells) were seeded at a density of 8,000 cells/well in 96-well tissue culture plates and treated for 24 hours with test substance at 25 μg/mL. Cytotoxicity was evaluated to remove false positives (CCK >80% viability). DCFH-DA (a fluorescent probe) was added to cells to detect ROS production and incubated at 37° C. for 25 minutes. After exposure to UV irradiation under the solar simulator (Sol-UV-6 Solar Simulator) with ultraviolet filter for 10 minutes, the fluorescence value was measured at 488 nm (excitation) and 525 nm (emission) by Multimode Reader. Vitamin C was used as positive control treated at 40 μg/mL with 43% reduction of ROS production. At 25 μg/mL, Rosmarinic acid decreased ROS generation by 24%, compared to the level of UV exposed HaCaT cells. Organic extracts (OE) were tested at 50 μg/mL, while aqueous extracts were tested at 100 μg/mL. Fractions or pure compounds were tested at 25 μg/mL in this assay.

TABLE 11
Inhibition against UV-ROS production in HaCaT cell

| | | | ROS | Viability | ROS | Viability | ROS | Viability |
|---|---|---|---|---|---|---|---|---|
| | | Sample | (%) | (%) | (%) | (%) | (%) | (%) |
| Latin name | Sample ID | description | 100 μg/mL | | 50 μg/mL | | 25 μg/mL | |
| Aloe | P01594-OE | Organic extract | / | / | 27 | 97 | / | / |
| barbadensis | | | | | | | | |
| Aloe vera | P00104-AE | Aqueous extract | 44 | 97 | / | / | / | / |
| Poria coccos | P02207-OE | Organic extract | / | / | 18 | 83 | / | / |
| Rosmarinus | P02630-AE | Aqueous extract | 42 | 103 | / | / | / | / |
| officinalis | | | | | | | | |
| Rosmarinus | P02630- | 10% MeOH | / | / | / | / | 24 | 93 |
| officinalis | 10M | fraction | | | | | | |
| Rosmarinus | Rosmarinic | >98% HPLC | / | / | / | / | −6 | 57 |
| officinalis | acid | purity | | | | | | |

### Example 15

### DNA Damage of Human Fibroblasts Induced by 30% Hydrogen Peroxide Assay

HSF cells (Human fibroblasts) were seeded in 96-well tissue culture plates and incubated at 37° C. and in 5% CO₂ and 95% air with test substances. Treated HSF cells were subjected to DNA damage by incubating with H₂O₂ at a concentration of 1 mM for 4 h, then immunostained for γH2AX, a phosphorylated histone that is a marker of DNA double strand breaks. DAPI was used to stain the cell nucleus. Pictures taken by Image Xpress and analyzed with Meta Xpress. Catechin (100 μg/ml) was used as positive control with 70% reduction of DNA damage, as assessed by quantification of γH2AX staining. production. Rosmarinic acid reduced DNA damage by 24% at 25 μg/mL.

### Example 16

### Aloe-Based Composition (UP360) Comprising Polysaccharides and Polyphenols Showed Dose Correlated Inhibition of HMGB1 and TNF in LPS Challenged Macrophages

One million RAW264.7 mouse macrophage-like cells were plated in serum-free media in 60 mm dishes with 1 μg/mL lipopolysaccharides (LPS) (except for the Control group). The UP360 composition comprising polysaccharides and polyphenols made in Example 9 was added in duplicate at the following concentrations: UP360—125, 250, and 500 μg/mL. The cells were treated for 24 hours before the media was aspirated and centrifuged in a 10,000 MWCO filter to concentrate. The media was run on SDS-PAGE and transferred to PVDF membranes for blotting for HMGB1 and TNF-α. The blots were stained with Ponceau S, and the densitometry was normalized to total protein amount.

The Aloe-based composition—UP360 showed dose correlated significant inhibition in HMGB1 and TNF-α in LPS challenged macrophages. From the western blot semi-quantitative data, it was found that when macrophages were challenged with LPS, there was a 1.1±0.17 and 9.8±0.33 relative band intensity for HMGB1 and TNF-α, respectively, for the vehicle control. In contrast, treating LPS-challenged macrophages with UP360 reduced the level of HMGB1 band intensity to 0.48±0.02, 0.27±0.01 and 0.17±0.01 for the 125, 250 and 500 μg/mL concentrations, respectively. Similarly, significantly reduced secretions of TNF-α i.e. 0.54±0.01 and 0.37±0.01 were found for the 250 and 500 μg/mL concentrations of UP360, respectively.

Macrophages were untreated (Control), treated only with LPS (Vehicle), or treated with LPS and the extract or composition at the indicated concentration (left) for 24 hours in duplicate before the media was collected and concentrated on a 10,000 MWCO filter. Concentrated media was run on SDS-PAGE and blotted for the indicated proteins (top). Densitometry was performed on the blots, normalized to the total Ponceau stain, and protein expression was calculated relative to the Control.

TABLE 12
Semi-quantitation of HMGB1 and TNF-α from UP360 Western
blot normalized to Ponceau S stain and relative to Control group:

| | LPS (1 μg/mL) | HMGB1 | TNF-α |
|---|---|---|---|
| Control | − | 1.0 +/− 0.07 | 1.0 +/− 0.005 |
| Vehicle | + | 1.1 +/− 0.17 | 9.8 +/− 0.33 |
| UP360 | | HMGB1 | TNF-α |
| 125 μg/mL | + | 0.48 +/− 0.02 | 6.6 +/− 0.49 |
| 250 μg/mL | + | 0.27 +/− 0.01 | 0.54 +/− 0.01 |
| 500 μg/mL | + | 0.17 +/− 0.01 | 0.37 +/− 0.01 |

### Example 17

### Aloe Based Composition (UP360) Comprising Polysaccharides and Polyphenols Showed Unexpected Synergistic Inhibitory Activity of HMGB1 and TNF-α

One million RAW264.7 mouse macrophage-like cells were plated in serum-free media in 60 mm dishes with 1μg/mL lipopolysaccharides (LPS) (except for the Control group). Plant extracts utilized for making UP360 in Example 9 were added in duplicate at the following concentrations: Aloe leaf gel powder—37.5, 75, and 150 μg/mL, Poria extract—75, 150, and 300 μg/mL, and Rosemary extract—12.5, 25, and 50 μg/mL. The three concentrations of Aloe, Poria, and Rosemary were equivalent to their concentrations in 125, 250, and 500 μg/mL UP360 in the example above. The cells were treated for 24 hours before the media was aspirated and centrifuged in a 10,000 MWCO filter to concentrate. The media was run on SDS-PAGE and transferred to PVDF membranes for blotting for HMGB1 and TNF-α. The blots were stained with Ponceau S, and the densitometry was normalized to total protein amount.

Supernatant from overnight LPS-challenged macrophages was utilized to evaluate for a possible unexpected inhibitory effect of extracts from Aloe, Poria and RA when formulated together in a specific ratio using Colby's method. In this method, a formulation with two or more materials is presumed to have unexpected synergy if the observed value of a certain endpoint measurement is greater than the hypothetically calculated expected values. When the expected and observed values are equal, there is an additive effect. However, when the observed value is lowered than the expected value, there is an unexpected inhibitory effect. In the current scenario, it was intended to have reduced levels of both inflammatory markers (HMGB1 and TNF-α) monitored in this assay to achieve a desired meaningful anti-inflammatory outcome.

TABLE 13
Semi-quantitation of Aloe, Poria and Rosemary Western blot
normalized to Ponceau S stain and relative to Control group:

| | | LPS (1 μg/mL) | HMGB1 | TNF-α |
|---|---|---|---|---|
| Control | | − | 1.0 +/− 0.03 | 1.0 +/− 0.23 |
| Vehicle | | + | 1.8 +/− 0.07 | 2.9 +/− 0.16 |
| Aloe leaf gel powder | | | HMGB1 | TNF-α |
| 37.5 | μg/mL | + | 0.36 +/− 0.05 | 0.38 +/− 0.07 |
| 75 | μg/mL | + | 0.28 +/− 0.004 | 0.42 +/− 0.02 |
| 150 | μg/mL | + | 0.37 +/− 0.008 | 0.53 +/− 0.02 |
| Poria extract | | | HMGB1 | TNF-α |
| 75 | μg/mL | + | 0.33 +/− 0.007 | 0.49 +/− 0.02 |
| 150 | μg/mL | + | 0.44 +/− 0.009 | 0.47 +/− 0.01 |
| 300 | μg/mL | + | 0.48 +/− 0.15 | 0.52 +/− 0.16 |
| Rosemary extract | | | HMGB1 | TNF-α |
| 12.5 | μg/mL | + | 1.7 +/− 0.21 | 2.2 +/− 0.34 |
| 25 | μg/mL | + | 1.3 +/− 0.007 | 2.1 +/− 0.01 |
| 50 | μg/mL | + | 1.6 +/− 0.16 | 1.7 +/− 0.22 |

TABLE 14
Unexpected Synergy for Aloe based Composition (UP360) in reduction of HGMB1 and TNF-α

| | | | | | |
|---|---|---|---|---|---|
| Dose | UP360 | Dose | UP360 | Dose | UP360 |
| μg/mL | 125 μg/mL | μg/mL | 250 μg/mL | μg/mL | 500 μg/mL |

HMGB1 Densitometry

| | | intensity | | | intensity | | | intensity |
|---|---|---|---|---|---|---|---|---|
| 38 | 3 Aloe (x) | 0.36 | 75 | 3 Aloe (x) | 0.28 | 150 | 3 Aloe (x) | 0.37 |
| 75 | 6 Poria (y) | 0.33 | 150 | 6 Poria (y) | 0.44 | 300 | 6 Poria (y) | 0.48 |
| 150 | 1 Rosmarinic | 1.70 | 25 | 1 Rosmarinic | 1.30 | 50 | 1 Rosmarinic | 1.60 |
| | Acid (z) | | | Acid (z) | | | Acid (z) | |
| | x + y + Z | 2.39 | | x + y + Z | 2.02 | | x + y + Z | 2.45 |
| | (xyz)/10000 | 0.00 | | (xyz)/10000 | 0.00 | | (xyz)/10000 | 0.00 |
| | ((xy) + (xz) + (yz))/ | 0.01 | | ((xy) + (xz) + (yz))/ | 0.01 | | ((xy) + (xz) + (yz))/ | 0.02 |
| | 100 | | | 100 | | | 100 | |
| 125 | Expected | 2.38 | 250 | Expected | 2.01 | 500 | Expected | 2.43 |
| | (3A6P1RA) | | | (3A6P1RA) | | | (3A6P1RA) | |
| | Observed | 0.48 | | Observed | 0.27 | | Observed | 0.17 |
| | (3A6P1RA) | | | (3A6P1RA) | | | (3A6P1RA) | |

TNF-α Densitometry

| | | intensity | | | intensity | | | intensity |
|---|---|---|---|---|---|---|---|---|
| 38 | 3 Aloe (x) | 0.38 | 75 | 3Aloe (x) | 0.42 | 150 | 3Aloe (x) | 0.53 |
| 75 | 6 Poria (y) | 0.49 | 150 | 6Poria (y) | 0.47 | 300 | 6Poria (y) | 0.52 |
| 150 | 1 Rosmarinic | 2.20 | 25 | 1 Rosmarinic | 2.10 | 50 | 1 Rosmarinic | 1.70 |
| | Acid (z) | | | Acid (z) | | | Acid (z) | |
| | x + y + Z | 3.07 | | x + y + Z | 2.99 | | x + y + Z | 2.75 |
| | (xyz)/10000 | 0.00 | | (xyz)/10000 | 0.00 | | (xyz)/10000 | 0.00 |
| | ((xy) + (xz) + (yz))/ | 0.02 | | ((xy) + (xz) + (yz))/ | 0.02 | | ((xy) + (xz) + (yz))/ | 0.02 |
| | 100 | | | 100 | | | 100 | |
| 125 | Expected | 3.05 | 250 | Expected | 2.97 | 500 | Expected | 2.73 |
| | (3A6P1RA) | | | (3A6P1RA) | | | (3A6P1RA) | |
| | Observed | 6.60 | | Observed | 0.54 | | Observed | 0.37 |
| | (3A6P1RA) | | | (3A6P1RA) | | | (3A6P1RA) | |

X = Aloe,
Y = Poria,
Z = Rosmarinic Acid;
Colby's equation for Expected values: (X + Y + Z) − (XY + XZ + YZ/100) + XYZ/10000

Macrophages were untreated (Control), treated only with LPS (Vehicle), or treated with LPS and the extract or composition at the indicated concentration (left) for 24 hours in duplicate before the media was collected and concentrated on a 10,000 MWCO filter. Concentrated media was run on SDS-PAGE and blotted for the indicated proteins (top). Densitometry was performed on the blots, normalized to the total Ponceau stain, and protein expression was calculated relative to the Control.

As documented in the table, we observed significantly reduced levels of both HMGB1 and TNF-α indicating the unexpected inhibitory effect of combining these medicinal plant materials yielding UP360. When the extracts were incubated at an individual concentration that would make up a dose of 125, 250 and 500 μg/mL UP360, the inhibitory effect for the standardized composition UP360 was greater than the theoretically calculated expected values for each dosages for both the markers except at the 125 μg/mL where the observed TNF-α value was higher than the expected. These values were 0.48 vs 2.38, 0.27 vs 2.01 and 0.17 vs 2.43 for the HMGB1 and 6.6 vs 3.05, 0.54 vs 2.97 and 0.37 vs 2.73 for the TNF at 125, 250 and 500 μg/mL, respectively. As such, the beneficial unexpected inhibitory effects of contemplated Aloe-based compositions, including UP360, comprising and, in some embodiments, consisting of polysaccharides and polyphenols treatment exceeded the expected outcome for reducing the secretion of HMGB1 and TNF-α to media.

### Example 18

### Animals and Housing

CD-1 mice were purchased from a USDA approved vendor. Male CD-1 mice at 8 weeks old were purchased form Charles River Laboratories, Inc. (Wilmington, Mass.). Animals were acclimated upon arrival and used for the study at the age of 11 weeks. At the time of the study animals average weigh 33.6±2.4 grams. They were housed in a temperature-controlled room (71-72° F.) on a 12-hour light-dark cycle and provided with feed and water ad libitum.

The animals were housed 3-5 per polypropylene mouse cage and individually identified by characteristically numbered on their tail. Each cage was covered with mouse wire bar lid and filtered mouse top (Allentown, N.J.). Individual cages were identified with a cage card indicating project number, test article, dose level, group, animal number and sex. The Harlan T7087 soft cob beddings was used and changed at least twice/week. mice were provided with fresh water and rodent chow diet #T2018 from Harlan (Harlan Teklad, 370W, Kent, Wash.) ad libitum.

### Example 19

### Lipopolysaccharide (LPS) Induced Sepsis Model as an Exogenous Assault Trigger Response

This model used a survival/mortality rate of animals as the end point measurement (Wang et al., 1999). LPS, an exogenous assault trigger, is an integral component of the outer membrane of gram-negative bacteria and a major contributing factor in the initiation of a generalized inflammatory process that could lead to endotoxic shock. It is a state mediated principally by macrophages/monocytes attributed to excessive production of several early phase cytokines such as TNF, IL-1, IL-6 and gamma interferon as well as a late stage mediator HMGB1. Following median lethal dose of LPS (25 mg/kg) administration dissolved in Phosphate buffered saline (PBS; Lifeline, Lot #07641), animals develop endotoxemia and HMGB1 would be detected in the serum at 8 hours and reach to a peak and plateau levels from 16 to 32 hours after LPS. If untreated, mice would start to die within 24 hours. In the current study, we monitored the mice for 4 days after LPS injection. The survival/mortality rate compared LPS +Sodium butyrate (SB; Aldrich, St. Louis, Mo.; lot #MKCG7272), LPS+Vehicle (0.5% CMC; Spectrum, New Brunswick, N.J.; lot #1IJ0127) and LPS+UP360 (Aloe based composition comprising polysaccharides and polyphenols made in Example 9). The following groups were included in the study:

TABLE 15
Details of Treatment groups

| Group | Treatment | Dose (mg/kg) | N |
|---|---|---|---|
| G1 | Normal control | 0 | 8 |
| G2 | Vehicle control (0.5% CMC) | 0 | 8 |
| G3 | Sodium Butyrate (SB) | 500 | 8 |
| G4 | Aloe based composition (UP360) | 500 | 8 |

In this model, mice were pretreated with UP360 illustrated in the Example 9 for a week (7 days) before lethal dose intraperitoneal injection of LPS (E. coli, 055:B5; Sigma, St. Louis, Mo.; Lot #081275) at 25 mg/kg with a 10 mL/kg PBS volume. Animals were observed hourly. Given the fact that Sodium butyrate improved LPS-induced injury in mice through suppression of HMGB1 release, we chose this compound as a positive control for our study (Li et al., 2018).

### Example 20

### Aloe Based Composition (UP360) Improved Animal Survival Rate Under a Lethal Dose of Toxin

Three hours following intraperitoneal injection of LPS, mice started to show early signs of endotoxemia. Exploratory behavior of the mice was progressively reduced and was accompanied by ruffled fur (piloerection), decreased mobility, lethargy, and diarrhea. While these signs and symptoms seemed to be present in all the treatment groups, the magnitude of severity was more pronounced in the vehicle-treated group.

Two mice from the vehicle-treated and one mouse from the positive control Sodium Butyrate (SB) groups were found deceased 24 hours after LPS injection. As seen in Table 16, the survival rates were determined for these groups and were found as 62.5% and 75%, respectively.

Mice treated with the Aloe based composition (UP360) had a 100% survival rate after 24 hours of LPS injection. Survival rates of 87.5%, 62.5% and 50% were observed for mice treated with contemplated Aloe-based compositions, including UP360, comprising and, in some embodiments, consisting of polysaccharides and polyphenols, Sodium Butyrate (SB) and Vehicle, respectively, 34 hours after LPS injection. Perhaps the most significant observation for Aloe-based composition (UP360)-treated mice was observed 48 hours after LPS injection. At this time point, there was only 12.5% survival rate for the vehicle-treated mice while Aloe-based composition (UP360)-treated mice showed a 62.5% survival rate. Even for the positive control group—SB, half of the animals were deceased at this time point. On the third day (72 hours after LPS injection), the survival rates for the groups were 62.5%, 50% and 12.5% for UP360, Sodium Butyrate and vehicle, respectively.

All mice in the vehicle control group were deceased after 82 hours of LPS injection, leaving 0% survival rate for this group. On the other hand, mice treated with Aloe-based composition (UP360) and positive control Sodium Butyrate (SB) showed a 62.5% and 50% survival rate and remained the same for 96 hours and 120 hours after LPS injection. These survival rates were statistically significant both for the Aloe-based composition (UP360) and the positive control (Table 16). Surviving animals in these groups showed progressive improvements in their wellbeing. Mice appeared physically better and gradually resumed to show normal behaviors. These data suggest that contemplated Aloe-based compositions, including UP360, comprising and, in some embodiments, consisting of polysaccharides and polyphenols could possibly be used as a prophylaxis and/or intervention dietary supplement to overcome a sudden surge of cytokines and HMGB1 ata time of sepsis.

TABLE 16
Aloe based composition (UP360) provided a 62.5%
survival rate from LPS induced endotoxemia & sepsis

| | | # of Death after LPS | | | | | | | | Survival | |
|---|---|---|---|---|---|---|---|---|---|---|---|
| Group | N | 24 hr | 32 hr | 34 hr | 48 hr | 58 hr | 72 hr | 82 hr | Total | Rate (%) | P-values |
| Control | 8 | 0 | 0 | 0 | 0 | 0 | 0 | 0 | 0 | 100 | — |
| Vehicle | 8 | 3 | 4 | 4 | 7 | 7 | 7 | 8 | 8 | 0 | — |
| UP360 | 8 | 0 | 1 | 1 | 3 | 3 | 3 | 3 | 3 | 62.5 | 0.001315 |
| Sodium | 8 | 2 | 3 | 3 | 4 | 4 | 4 | 4 | 4 | 50 | 0.014806 |
| Butvrate | | | | | | | | | | | |

The survival rate was calculated as: 100 − [(deceased mice/total number of mice) × 100]%.

### Example 21

### Comparison of Aloe-Based Composition (UP360) and Its Constituents in LPS-Induced Cepsis Model

The merit of combining Aloe, Poria, and Rosmarinic acid (RA) to yield UP360 comprising polysaccharides and polyphenols at a specific ratio demonstrated in Example-9 was evaluated in Lipopolysaccharide (LPS)-induced endotoxemia. Male CD-1 mice (n=13) were treated with Aloe, Poria, and Rosmarinic acid (RA) utilized for making UP360 in Example 9 at 150 mg/kg, 300 mg/kg and 50 mg/kg, respectively, for 7 days before LPS injection. On the 8^{th} day, mice were injected with 25 mg/kg LPS dissolved in PBS at 10 mL/kg intraperitoneally. Mice in the UP360-treated group received a daily dose of UP360 at 500 mg/kg. All mice continued to receive the respective treatment daily for the duration of the study. Following median lethal dose of LPS (25 mg/kg) i.p. administration, animals are expected to develop sepsis within a few hours. If untreated, mice would start to die within 24 hours. Animals were observed hourly. In the current study, we monitored the mice for 6 days after LPS injection. The survival rate compared LPS+Sodium butyrate (SB), LPS+Vehicle (0.5% CMC), LPS+UP360, LPS+Aloe, LPS+Poria and LPS+Rosmarinic acid. Normal control animals received only PBS intraperitoneally and gavaged only with the carrier vehicle 0.5% CMC. Given the fact that Sodium butyrate (SB) improved LPS-induced injury in mice through suppression of HMGB1 release, we chose this compound as a positive control for our study (Li et al., 2018).

The survival rate and mortality rate of the contemplated Aloe-based compositions, including UP360, comprising and, in some embodiments, consisting of polysaccharides and polyphenols was compared with those dosages of individual extracts as they appear in the formulation to find out potential additive, antagonist or synergistic effects in combination using Colby's equation (Colby, 1967). For the blending of these plant extracts to have unexpected synergy, the observed inhibition needs to be greater than the calculated value.

Few hours post intraperitoneal injection of LPS, mice started to show early signs of sepsis. Exploratory behavior of mice was progressively reduced and was accompanied by ruffled fur (piloerection), decreased mobility, lethargy, diarrhea, and shivering accompanied by closed eyelids for some. While these signs and symptoms were present in all the treatment groups, the magnitude of severity was more severe in the vehicle treatment group.

As seen in Table 17, 18, & 19, no death was observed for mice treated with the Aloe-based composition (UP360) for the first 36 hours post-model induction. Treatment with the Aloe-based composition (UP360) resulted in a 100% survival rate for the first 36 hours. On the other hand,

TABLE 17
Time course of survival and mortality in LPS induced sepsis

| | | | Number of deceased animals post LPS | | | | | | | | | | | | |
|---|---|---|---|---|---|---|---|---|---|---|---|---|---|---|---|
| | Dose | | 24 | 36 | 48 | 60 | 72 | 96 | 120 | 144 | | | MR | SR | |
| Group | (mg/kg) | N | hpi | hpi | hpi | hpi | hpi | hpi | hpi | hpi | Deceased | Survived | (%) | (%) | P-value |
| Control | 0 | 13 | 0 | 0 | 0 | 0 | 0 | 0 | 0 | 0 | 0 | 13 | 0.0 | 100 | 0.000 |
| Vehicle | 0 | 13 | 4 | 2 | 5 | 0 | 0 | 0 | 0 | 0 | 11 | 2 | 84.6 | 15.4 | — |
| SB | 500 | 13 | 2 | 4 | 1 | 1 | 0 | 1 | 0 | 0 | 9 | 4 | 69.2 | 30.8 | 0.27 |
| UP360 | 500 | 13 | 0 | 0 | 2 | 0 | 1 | 1 | 0 | 0 | 4 | 9 | 30.8 | 69.2 | 0.001 |
| Aloe | 150 | 13 | 2 | 2 | 4 | 1 | 0 | 0 | 0 | 1 | 10 | 3 | 76.9 | 23.1 | 0.35 |
| Poria | 300 | 13 | 1 | 2 | 3 | 1 | 0 | 0 | 0 | 0 | 7 | 6 | 53.9 | 46.2 | 0.06 |
| RA | 50 | 13 | 2 | 2 | 4 | 0 | 0 | 0 | 0 | 0 | 8 | 5 | 61.5 | 38.5 | 0.18 |

The survival rate was calculated as: 100 − [(deceased mice/total number of mice) × 100]%.
RA—Rosmarinic Acid.

TABLE 18
Survival rate of LPS-induced septic mice treated with Aloe-based composition UP360

| | Survival rate (%) | | | | | | | | |
|---|---|---|---|---|---|---|---|---|---|
| Group | 0 hr | 24 hr | 36 hr | 48 hr | 60 hr | 72 hr | 96 hr | 120 hr | 144 hr |
| Normal control | 100 | 100 | 100 | 100 | 100 | 100 | 100 | 100 | 100 |
| Vehicle | 100 | 69.2 | 53.8 | 15.4 | 15.4 | 15.4 | 15.4 | 15.4 | 15.4 |
| Sodium Butyrate 500 mg/kg | 100 | 84.6 | 53.8 | 46.2 | 38.5 | 38.5 | 30.8 | 30.8 | 30.8 |
| UP360 500 mg/kg | 100 | 100.0 | 100.0 | 84.6 | 84.6 | 76.9 | 69.2 | 69.2 | 69.2 |
| Aloe 150 mg/kg | 100 | 84.6 | 69.2 | 38.5 | 30.8 | 30.8 | 30.8 | 30.8 | 23.1 |
| Poria 300 mg/kg | 100 | 92.3 | 76.9 | 53.8 | 46.2 | 46.2 | 46.2 | 46.2 | 46.2 |
| RA 50 me/ke | 100 | 84.6 | 69.2 | 38.5 | 38.5 | 38.5 | 38.5 | 38.5 | 38.5 |

The survival rate was calculated as: 100 − [(deceased mice/total number of mice) × 100]%.
RA—Rosmarinic Acid

TABLE 19
Mortality rate of LPS-induced septic mice treated with Aloe-based composition UP360

| | Mortality rate (%) | | | | | | | | |
|---|---|---|---|---|---|---|---|---|---|
| Group | 0 hr | 24 hr | 36 hr | 48 hr | 60 hr | 72 hr | 96 hr | 120 hr | 144 hr |
| Normal control | 0 | 0 | 0 | 0 | 0 | 0 | 0 | 0 | 0 |
| Vehicle | 0.0 | 30.8 | 46.2 | 84.6 | 84.6 | 84.6 | 84.6 | 84.6 | 84.6 |
| Sodium Butyrate 500 mg/kg | 0.0 | 15.4 | 46.2 | 53.8 | 61.5 | 61.5 | 69.2 | 69.2 | 69.2 |
| UP360 500 mg/kg | 0.0 | 0.0 | 0.0 | 15.4 | 15.4 | 23.1 | 30.8 | 30.8 | 30.8 |
| Aloe 150 mg/kg | 0.0 | 15.4 | 30.8 | 61.5 | 69.2 | 69.2 | 69.2 | 69.2 | 76.9 |
| Poria 300 mg/kg | 0.0 | 7.7 | 23.1 | 46.2 | 53.8 | 53.8 | 53.8 | 53.8 | 53.8 |
| RA 50 mg/kg | 0.0 | 15.4 | 30.8 | 61.5 | 61.5 | 61.5 | 61.5 | 61.5 | 61.5 |

The Mortality rate was calculated as: 100 − survival rate.
RA—Rosmarinic Acid

the constituents such as Aloe, Poria and Rosmarinic acid-treated mice experienced 69.2%, 76.9% and 69.2% survival rates, respectively. In this time frame (36 hours post injection—hpi), the vehicle group showed a 53.8% survival rate. The highest mortality for each group was observed on the 2nd day post LPS (48hpi).

Mortality rates of 61.5%, 46.2% and 61.5% were observed for Aloe, Poria and Rosmarinic acid-treated mice, respectively, 48 hours post-LPS. Mice in the Aloe-based composition (UP360) group experienced only a 15.4% mortality rate. The vehicle-treated mice showed an 84.6% mortality rate as of 48 hours post-LPS and remained the same for the rest of the study period. On the third day (72 hours after LPS injection), the survival rates for the treatment groups were 76.9%, 30.8%, 46.2% and 38.5% for aloe composition (UP360), Aloe, Poria and Rosmarinic acid, respectively. The positive control showed 38.5% survival rate at this time frame.

At the end of the 6^{th} day (144hpi), while the Aloe-based composition (UP360) showed 69.2% survival rate, mice in the Aloe, Poria and RA group showed a 23.1%, 46.2% and 38.5% survival rate, respectively (Table 17, 18, 19). The survival rate observed for the Aloe-based composition was statistically significantly increased compared to the vehicle-treated group. The SB group finished the study with a 30.8% survival rate. Surviving animals in the groups showed progressive improvements in their wellbeing. Mice appeared physically better and gradually resumed to show normal exploratory behaviors.

### Example 22

### Unexpected Synergy in Decreasing Mortality Rate was Observed for Aloe-Based Composition (UP360) Comprising Polysaccharides and Polyphenols

This LPS-induced survival study was utilized to evaluate possible synergy or unexpected effects of extracts from Aloe, Poria and Rosmarinic acid (RA) when formulated together in a specific ratio using Colby's method. When mice were given the Aloe-based composition (UP360) composition at a dose of 500 mg/kg, the mortality rates were lower than the theoretically calculated expected values at each time point analyzed (Table 20). For example, while the expected mortality rate at 24 and 60 hours post-LPS injection was 33.9% and 94.5%, respectively, the actual observed mortality rate for Aloe-based composition (UP360) was0% and 15.4%, respectively. These findings suggest that formulating these three standardized extracts from Aloe, Poria and RA at a specific ratio of 3:6:1 has a far greater benefit than using Aloe, Poria or RA extract alone in prolonging the life of study subjects at the time of sepsis.

TABLE 20
Unexpected Synergy in decreasing mortality rate
was observed for Aloe based composition UPS60

Mortality rate (%)

| Hours post | | | | | Observed |
|---|---|---|---|---|---|
| LPS | X | Y | Z | Expected | (UP360) |
| 24 | 15.4 | 7.7 | 15.4 | 33.9 | 0 |
| 36 | 30.8 | 23.1 | 30.8 | 63.2 | 0 |
| 48 | 61.5 | 46.2 | 61.5 | 92 | 15.4 |
| 60 | 69.2 | 53.9 | 61.5 | 94.5 | 15.4 |
| 72 | 69.2 | 53.9 | 61.5 | 94.5 | 23.1 |
| 96 | 69.2 | 53.9 | 61.5 | 94.5 | 30.8 |
| 120 | 69.2 | 53.9 | 61.5 | 94.5 | 30.8 |
| 144 | 76.9 | 53.9 | 61.5 | 95.9 | 30.8 |

X = Aloe,
Y = Poria,
Z = Rosmarinic Acid;
Colby's equation for Expected mortality rate: (X + Y + Z) − (XY + XZ + YZ/100) + XYZ/10000

It was expected that 95.9% of the study subjects to be deceased at the end of the observation period, whereas the actual mortality rate for the Aloe-based composition (UP360) was found as 30.8%.

As such, the merit of combining Aloe, Poria and RA extracts was evaluated and confirmed using Colby's equation in this LPS-induced survival study. In this method, a formulation with two or more materials is presumed to have unexpected synergy if the observed value of a certain endpoint measurement is greater than the hypothetically calculated expected values. Mortality rate values of these medicinal plants at 24, 36, 48, 60, 72, 96, 120 and 144 hours after LPS injection were used to determine the calculated efficacy values and compared to the observed mortality rate values of the Aloe-based composition (UP360) at the specified time points. In the present study, we found unexpected synergy as a result of the combination of Aloe, Poria and RA extracts. The beneficial effects of Aloe-based composition (UP360) treatment exceeded the expected outcome for mortality rate compared to the extracts given alone. At the end of the observation period, there was a 30.8% mortality rate for Aloe based composition (UP360), while there was 76.9, 53.9% and 61.5% mortality rate for each Aloe, Poria and RA extract treatment groups, respectively, suggesting the unexpected synergistic activities of these botanical extracts comprising polysaccharides and polyphenols in protecting cytokine storm and hence decreasing mortality rate of patients at the time of sepsis (Table 20).

### Example 23

### Efficacy of Aloe-Based Composition (UP360) on Mitigating Lipopolysaccharide (LPS)-Induced Acute Inflammatory Lung Injury in Rats—As an Exogenous Assault Trigger Response

The study was designed to evaluate the direct impact of an Aloe-based composition (UP360) made in the Example 9 in alleviating LPS-induced acute lung injury administered orally at 500 mg/kg and 250 mg/kg. Acute lung injury is a clinical syndrome characterized by alveolar epithelial cells and capillary endothelial cell damage resulting in diffuse lung injury as seen in acute respiratory distress syndrome (ARDS). In this study, we treated rats with the test materials orally for 7 days before model induction with LPS. On the 8^{th} day, an hour after oral treatment, LPS was instilled intratracheally (i.t.) at 10 mg/kg dissolved in 0.1 mL/100 g PBS to each rat. The normal control rats received the same volume i.t. PBS only.

TABLE 21
Study groups

| Group | Treatment | Dose (mg/kg) | N |
|---|---|---|---|
| G1 | Normal control | 0 | 7 |
| G2 | Vehicle control | 0 | 10 |
| G3 | Sodium Butyrate | 500 | 10 |
| G4 | UP360 -High dose | 500 | 10 |
| G5 | UP360-Low dose | 250 | 10 |

LPS is known to induce systemic and pulmonary responses, leading to accumulation of proinflammatory cells, including neutrophils and macrophages and proinflammatory cytokines such as IL-1, IL-8, IL-6, MIP-2/CINC-3 and TNF-α. This causes pulmonary interstitial and alveolar edema, and epithelial cell damage wherein HMGB1 is secreted actively by macrophages and monocytes and/or passively released from necrotic cells.

We sacrificed surviving animals 24 hours after intratracheal LPS administration to the rats. At necropsy, collected bronchoalveolar lavage (BAL) by intratracheal injection of 1.5 mL PBS into the right lobe followed by gentle aspiration at least 3 times. Pooled recovered fluid, centrifuged at 1500 rpm for 10 min at 4 ° C., and was used to measure cytokines (e.g. IL-6) and pulmonary protein levels. This same right lobe was collected for tissue homogenization from each rat for MIP-2/CINC-3 protein analysis. The left lobe was fixed with formalin and submitted for histopathology evaluation to Nationwide Histology for analysis by a certified pathologist. Serum collected at necropsy was used to measure cytokines such as TNF-α and IL-1β. Following intratracheal instillation of LPS at 10 mg/kg, all animals survived for 24 hours post-challenge. Here, we have compiled key cytokines and chemoattractants believed to be involved in the pathology of acute pulmonary infection and data from the histopathology analysis in the following examples.

### Example 24

### Aloe-Based Composition (UP360) Showed a Dose-Correlated Statistically Significant Reduction in Serum TNF-α

The presence of TNF-α in undiluted rat serum from Example 23 was measured using the rat TNF-α Quantikine ELISA kit from R&D Systems (product#: RTA00) as follows: undiluted serum was added to a microplate coated with TNF-α antibody. After 2 hours at room temperature, TNF-α in serum was bound to the plate and the plate was thoroughly washed. Enzyme-conjugated TNF-α antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of TNF-α was calculated based on the absorbance readings of a TNF-α standard curve.

As seen in Table 22, a statistically significant surge in serum TNF-α was observed for vehicle-treated rats challenged with LPS. This increase was significantly reduced when rats were treated with contemplated Aloe-based compositions, including UP360, comprising and, in some embodiments, consisting of polysaccharides and polyphenols. Statistically significant and dose-correlated reductions were observed for rats treated with the Aloe-based composition (UP360) at 500 mg/kg and 250 mg/kg orally. These reductions in serum TNF-α level were calculated against the vehicle control and were found to be 91.9% and 73.6% for Aloe-based composition (UP360) treated groups at 500 mg/kg and 250 mg/kg, respectively. The positive control, Sodium Butyrate (SB), showed a statistically significant (67.9%) reduction in serum TNF-α level.

TABLE 22
Effect of Aloe based composition (UP360) on serum TNF-α level.

| | Dose | | Mean ± SD | |
|---|---|---|---|---|
| Group | (mg/kg) | N | (pg/mL) | p-value |
| Normal control | 0 | 7 | −1.27 ± 0.93 | 0.000001 |
| Vehicle control | 0 | 10 | 10.43 ± 2.48 | — |
| Sodium Butyrate | 500 | 10 | 3.35 ± 1.73 | 0.000001 |
| UP360 | 500 | 10 | 0.85 ± 1.08 | 0.000001 |
| UP360 | 250 | 10 | 2.75 ± 1.22 | 0.000001 |

### Example 25

### Aloe-Based Composition (UP360) Showed a Dose-Correlated Statistically Significant Reduction in Serum IL-1β

The presence of IL-1β in undiluted rat serum from Example 23 was measured using the Rat IL-1β Quantikine ELISA kit from R&D Systems (product#: RLBOO) as follows: undiluted serum was added to a microplate coated with IL-1β antibody. After 2 hours at room temperature, IL-1β in serum was bound to the plate and the plate was thoroughly washed. Enzyme-conjugated IL-1β antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-1β was calculated based on the absorbance readings of an IL-1β standard curve.

Here again, dose-correlated and statistically significant reduction of IL-1β was observed for rats treated with the contemplated Aloe-based compositions, including UP360, comprising and, in some embodiments, consisting of polysaccharides and polyphenols. A statistically significant increase in serum level of IL-1β was observed for LPS-induced acute lung injury rats treated with vehicle. Rats treated with the Aloe-based composition (UP360) showed 80.0% and 63.0% reductions in the IL-1β level when administered at oral dosages of 500 mg/kg and 250 mg/kg, respectively (Table 23). The Sodium Butyrate (SB) group showed a 65.3% reduction in serum IL-1β. The demonstrated serum IL-1β reductions were statistically significant for both the Aloe-based composition (UP360) and the Sodium Butyrate (SB) groups

TABLE 23
Effect of Aloe based composition (UP360) on serum IL-1β level.

| | Dose | | Mean ± SD | |
|---|---|---|---|---|
| Group | (mg/kg) | N | (pg/mL) | p-value |
| Normal control | 0 | 7 | −0.14 ± 4.20 | 0.000001 |
| Vehicle control | 0 | 10 | 65.09 ± 13.24 | — |
| Sodium Butyrate | 500 | 10 | 22.58 ± 9.46 | 0.000001 |
| UP360 | 500 | 10 | 13.01 ± 4.79 | 0.000001 |
| UP360 | 250 | 10 | 24.07 ± 7.74 | 0.000001 |

### Example 26

### Aloe Based Composition (UP360) Showed Dose-Correlated and Statistically Significant Reduction IL-6 Level in Broncho-Alveolar Lavage (BAL)

The presence of IL-6 in undiluted rat broncho-alveolar lavage (BAL) from Example 23 was measured using the Rat IL-6 Quantikine ELISA kit from R&D Systems (product #: R6000B) as follows: undiluted BAL was added to a microplate coated with IL-6 antibody. After 2 hours at room temperature, IL-6 in the BAL was bound to the plate and the plate was thoroughly washed. Enzyme-conjugated IL-6 antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-6 was calculated based on the absorbance readings of an IL-6 standard curve.

In agreement with the TNF-α and IL-1β data above, the Aloe-based composition (UP360 made in Example 9) showed a dose-correlated statistically significant reduction in the level of

BAL IL-6. While the higher dose (500 mg/kg) resulted in a 82.0% reduction in the level of BAL IL-6, the lower dose (250 mg/kg) showed a 51.0% reduction in the level of BAL IL-6 (Table 24). The reduction was statistically significant for the contemplated Aloe-based compositions, including UP360, comprising and, in some embodiments, consisting of polysaccharides and polyphenols at the high dose when compared to the vehicle-treated acute lung injury rats. A strong trend was also observed for the low dose of the Aloe based composition (UP360) (i.e. p=0.087). The Sodium Butyrate (SB) group showed a statistically non-significant 37.7% reduction of BAL IL-6 relative to the vehicle-treated disease model.

TABLE 24
Effect of Aloe based composition (UP360) on BAL IL-6 level.

| | Dose | | Mean ± SD | |
|---|---|---|---|---|
| Group | (mg/kg) | N | (pg/mL) | p-value |
| Normal control | 0 | 7 | 66.41 ± 4.86 | 0.000001 |
| Vehicle control | 0 | 10 | 3103.95 ± 3057.13 | — |
| Sodium Butyrate | 500 | 10 | 1933.30 ± 1744.23 | 0.27 |
| UP360 | 500 | 10 | 558.94 ± 354.88 | 0.0005 |
| UP360 | 250 | 10 | 1522.03 ± 1407.62 | 0.087 |

### Example 27

### Aloe-Based Composition (UP360) Treatment Produced a Statistically Significant Reduction in CINC-3

CINC-3/macrophage inflammatory protein 2 (MIP-2) belongs to the family of chemotactic cytokines known as chemokines. MIP-2 belongs to the CXC chemokine family, is named CXCL2 and acts through binding of CXCR1 and CXCR2. It is produced mainly by macrophages, monocytes and epithelial cells and is responsible for chemotaxis to the source of inflammation and activation of neutrophils.

50 μL of each rat lung homogenate sample from Example 23 (10 per group for Vehicle, Sodium Butyrate (SB), UP360 Low dose, UP360 High dose, 7 per group for Control) and 50 μL of assay diluent buffer were added to the wells of a 96-well microplate coated with monoclonal CINC-3 antibody and allowed to bind for 2 hours. The plate was subjected to 5 washes before an enzyme-linked polyclonal CINC-3 was added and allowed to bind for 2 hours. The wells were washed another 5 times before a substrate solution was added to the wells and the enzymatic reaction was allowed to commence for 30 minutes at room temperature protected from light. The enzymatic reaction produced a blue dye that changed to yellow with the addition of the stop solution. The absorbance of each well was read at 450 nm (with a 580 nm correction) and compared to a standard curve of CINC-3 in order to approximate the amount of CINC-3 in each rat lung homogenate sample.

The daily oral treatment of the contemplated Aloe-based compositions, including UP360, comprising and, in some embodiments, consisting of polysaccharides and polyphenols (UP360 made in Example 9) at 500 mg/kg for a week caused a statistically significant reduction in a cytokine induced neutrophil chemoattractant in LPS-induced acute lung injury (Table 25). The level of CINC-3 in the normal control rats receiving only the PBS intratracheally was near zero. In contrast, intratracheal LPS-induced acute lung injury rats treated with the carrier vehicle showed an average lung homogenate level of CINC-3 at 563.7±172.9 pg/mL. This level was reduced to an average value of 280.92±137.84 pg/mL for the 500 mg/kg Aloe based composition (UP360) treated rats. This 50.2% reduction in CINC-3 level for the rats treated with 500 mg/kg of Aloe based composition (UP360) was statistically significant when compared to vehicle-treated disease model. The lower dose Aloe-based composition (UP360) produced a moderate (i.e. 27.6%) reduction in CINC3 level when compared to the vehicle control group. The Sodium Butyrate (SB) group only had a marginal (i.e. 17.7%) reduction in lung homogenate CINC-3 level in comparison to the vehicle-treated rats.

TABLE 25
Effect of Aloe based composition (UP360) on
lung homogenate MIP-2/CINC-3 activity level.

| | Dose | | Mean ± SD | |
|---|---|---|---|---|
| Group | (mg/kg) | N | (pg/mL) | p-value |
| Normal control | 0 | 7 | −4.21 ± 2.38 | 0.0000 |
| Vehicle control | 0 | 10 | 563.71 ± 194.81 | — |
| Sodium Butyrate | 500 | 10 | 464.00 ± 220.32 | 0.2980 |
| UP360 | 500 | 10 | 280.92 ± 137.84 | 0.0020 |
| UP360 | 250 | 10 | 408.29 ± 209.20 | 0.1028 |

### Example 28

### Aloe-Based Composition (UP360) Reduced Total Protein in Broncho-Alveolar Lavage (BAL)

The amount of total proteins in the broncho-alveolar lavage (BAL) samples from Example 23 was measured using the Pierce BCA Protein Assay kit from ThermoFisher Scientific (product #: 23225) as follows: BAL was diluted 1:5, mixed with bicinchoninic acid (BCA) reagent in a microplate, and incubated at 37° C. for 30 minutes. Absorbance was read at 580 nm, and protein concentration in BAL was calculated based on the absorbance readings of a bovine serum albumin standard curve.

A 3-fold increase in the level of lung total protein from the BAL was found in the LPS-induced acute lung injury rats treated with vehicle compared to the normal control rats. Daily oral treatment of rats for a week with the contemplated Aloe-based compositions, including UP360, comprising and, in some embodiments, consisting of polysaccharides and polyphenols (UP360 made in Example 9) at 500 mg/kg and 250 mg/kg resulted in a 40.1% (p=0.12 vs vehicle) and 38.3% (p=0.17) reduction, respectively, in the content of BAL total proteins when compared to vehicle-treated LPS-induced acute lung injury rats (Table 26). The positive control Sodium Butyrate (SB) group had a 30.2% (p=0.27) reduction in the level of BAL total proteins relative to the vehicle-treated LPS-induced acute lung injury rats.

TABLE 26
Effect of Aloe based composition (UP360) on BAL protein level.

| | Dose | | Mean ± SD | |
|---|---|---|---|---|
| Group | (mg/kg) | N | (μg/mL) | p-value |
| Normal control | 0 | 7 | 1488.88 ± 322.01 | 0.00367209 |
| Vehicle control | 0 | 10 | 4214.86 ± 3311.32 | — |
| Sodium Butyrate | 500 | 10 | 2940.14 ± 2092.32 | 0.265745965 |
| UP360 | 500 | 10 | 2526.23 ± 1497.78 | 0.124589339 |
| UP360 | 250 | 10 | 2599.89 ± 691.39 | 0.168377963 |

### Example 29

### Aloe-Based Composition (UP360) Showed Statistically Significant Reduction in C Reactive Protein in Broncho-Alveolar Lavage (BAL)

The presence of C Reactive Protein (CRP) in rat BAL diluted 1:1,000 was measured using the C Reactive Protein (PTX1) Rat ELISA kit from Abcam (product #: ab108827) as follows: 1:1,000 diluted BAL was added to a microplate coated with CRP antibody. After 2 hours on a plate shaker at room temperature, CRP in BAL was bound to the plate and the plate was thoroughly washed. Biotinylated C Reactive Protein Antibody was added to the plate and allowed to bind for 1 hour on a plate shaker at room temperature. The washing was repeated, and Streptavidin-Peroxidase Conjugate was added to the plate. After incubating for 30 minutes at room temperature, washing was repeated, and chromogen substrate was added. After developing for 10 minutes at room temperature, a stop solution was added, and the absorbance was read at 450 nm. The concentration of CRP was calculated based on the absorbance readings of a CRP standard curve.

A statistically significant 5.6-fold increase in BAL CRP level was observed in the LPS-induced acute lung injury rats treated with vehicle compared to the normal control rats. Oral treatment of rats for a week with contemplated Aloe-based compositions, including UP360, comprising and, in some embodiments, consisting of polysaccharides and polyphenols made in Example 9 at 500 mg/kg reduced the level of BAL CRP by 38.2% (p=0.06) relative to the vehicle-treated disease model (Table 27). The positive control Sodium Butyrate (SB) and the low dose of UP360 group resulted in minimal changes in CRP level compared to the vehicle-treated diseased rats.

TABLE 27
Effect of Aloe based composition (UP360) on BAL CRP level

| | Dose | | Mean ± SD | |
|---|---|---|---|---|
| Group | (mg/kg) | N | (pg/mL) | p-value |
| Normal control | 0 | 7 | 4344.5 ± 3321.6 | 0.0002 |
| Vehicle control | 0 | 10 | 24302.8 ± 8826.1 | — |
| Sodium Butyrate | 500 | 10 | 20093.5 ± 8826.1 | 0.35 |
| UP360 | 500 | 10 | 15012.0 ± 9274.3 | 0.06 |
| UP360 | 250 | 10 | 20999.6 ± 6421.2 | 0.42 |

### Example 30

### Aloe-Based Composition (UP360) Showed a Statistically Significant Reduction of IL-10 in Broncho-Alveolar Lavage

The presence of IL-10 in undiluted broncho-alveolar lavage (BAL) samples from Example 23 was measured using the Rat IL-10 Quantikine ELISA kit from R&D Systems (product #: R1000) as follows: undiluted BAL was added to a microplate coated with IL-10 antibody. After 2 hours at room temperature, IL-10 in serum was bound to the plate and the plate was thoroughly washed. Enzyme-conjugated IL-10 antibody was added to the plate and allowed to bind for 2 hours at room temperature. The washing was repeated, and enzyme substrate was added to the plate. After developing for 30 minutes at room temperature, a stop solution was added, and the absorbance was read at 450 nm. The concentration of IL-10 was calculated based on the absorbance readings of an IL-10 standard curve.

The anti-inflammatory IL-10 level was measured in the BAL of diseased rats sacrificed 24 hours post intratracheal instillation of LPS following a daily oral treatment of the contemplated aloe-based compositions, including UP360, comprising and, in some embodiments, consisting of polysaccharides and polyphenols (UP360 made in Example 9) at 500 mg/kg and 250 mg/kg for 7 days pre-induction. Often, the level of IL-10 corresponds with the severity of infection and inflammatory response need by the host at the time of infection or injury. As seen in Table 28, the level of IL-10 was found significantly increased (i.e. 80-fold in comparison with the normal control rats) for the vehicle-treated rats, indicating the high severity of the acute lung injury. In contrast, rats in the Aloe-based composition comprising, and in some instances consisting of polysaccharides and polyphenols (UP360) groups showed a dose-correlated reduction of IL-10 in the BAL. These reductions were calculated and determined to be 73.2% and 41.0% for the Aloe-based composition (UP360) at 500 mg/kg and 250 mg/kg, respectively. The reduction was statistically significant for the high dose (500 mg/kg) of Aloe based composition (UP360) at p≤0.05. At least for this specific model, the reduction in anti-inflammatory cytokine as a result of Aloe based composition (UP360) treatment could be explained by the fact that there may be a dampening effect in inflammatory response by the host due to mitigation of disease severity. Reinforcing this hypothesis, the Aloe-based composition comprising, and in some instances consisting of, polysaccharides and polyphenols (UP360) caused a statistically significant reduction in inflammatory cytokines such as IL-10, IL-6 and TNF-α, leading to a robust inflammatory response, rendering the need for anti-inflammatory cytokines, such as IL-10, less vital to the host. In fact, the level of IL-10 was nearly zero for the normal control group, suggesting induction of anti-inflammatory cytokines are based on presence and/or severity of acute lung injury.

TABLE 28
Effect of Aloe based composition (UP360) on BAL IL-10 level

| | Dose | | Mean ± SD | |
|---|---|---|---|---|
| Group | (mg/kg) | N | (pg/mL) | p-value |
| Normal control | 0 | 7 | 2.63 ± 8.35 | 0.004 |
| Vehicle control | 0 | 10 | 207.77 ± 171.33 | — |
| Sodium Butyrate | 500 | 10 | 154.84 ± 159.63 | 0.48 |
| UP360 | 500 | 10 | 55.64 ± 40.53 | 0.02 |
| UP360 | 250 | 10 | 122.6 ± 83.76 | 0.18 |

### Example 31

### Aloe-Based Composition (UP360) Reduced Pulmonary Edema and Overall Lung Damage Severity

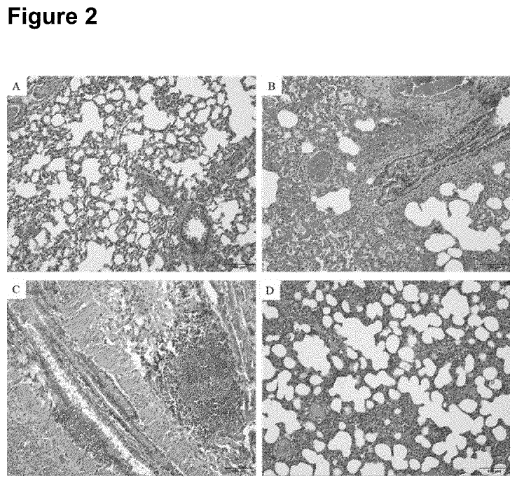

The severity of lung damage as a result of intratracheal LPS in Example 23 was assessed using H&E stained lung tissue. The left lobe of the lung was used for the histopathology analysis. As seen in the Table 29 and FIG. 2 below, rats in the vehicle-treated group showed a statistically significant increase in the severity of lung damage (3.5-fold increase), pulmonary edema (2.5-fold increase) and infiltration of polymorphonuclear (PMN/PMC) cells (2.4-fold increase). Daily oral treatment of rats for a week with the high dose of the Aloe-based composition (UP360 made in Example 9) at 500 mg/kg resulted a statistically significant 37.9% reduction in the overall lung damage severity when compared to vehicle-treated LPS-induced acute lung injury rats (Table 29, FIG. 2). Similarly, a strong, statistically significant reduction in pulmonary edema (37% reduction) was observed for the high dose of the Aloe-based composition (UP360) when compared to the vehicle-treated rats. A positive trend in the reduction of PMN infiltration was also observed for rats treated with the high dose of the Aloe-based composition (UP360) comprising, and in some instances consisting of polysaccharides and polyphenols. The positive control, Sodium Butyrate (SB), group caused minimal changes in the histopathology evaluation relative to the vehicle-treated diseased rats.

TABLE 29
Histopathology data for Aloe based composition
(UP360) from acute lung injury in rats

| | Dose | | Overall lung | Pulmonary | Infiltration |
|---|---|---|---|---|---|
| Group | (mg/kg) | N | tissue Severity ^{a} | Edema ^{b} | of PMC ^{c} |
| N. Control | 0 | 7 | 0.93 ± 0.49*** | 1.21 ± 0.52*** | 1.14 ± 0.58** |
| Vehicle | 0 | 9 | 3.22 ± 0.58 | 3.00 ± 0.67 | 2.72 ± 0.82 |
| SB | 500 | 10 | 3.05 ± 0.42 | 2.35 ± 0.95 | 2.75 ± 0.78 |
| UP360 | 500 | 9 | 2.00 ± 0.94* | 1.89 ± 0.57** | 2.06 ± 0.83 |
| UP360 | 250 | 10 | 3.30 ± 0.60 | 2.75 ± 0.75 | 3.30 ± 0.60 |

*P ≤ 0.05;
**P ≤ 0.001;
***P ≤ 0.00001;
SB—Sodium Butyrate;
PMN—polymorphonuclear cells
^{a} Overall Severity: Norm, mim-mild, mod, severe, ext. severe. Focal, m-focal, regional, reg. ext coalesing, diffuse, Score 0-4.
^{b} Acute Exudative changes: alv, duct & bronch, alv wall & Int edema, congestion, hemorrhage perivasc, alv sac, edema, fibr exud, hemorr alv sac, alv duct thicken dt Hyal membrane type I loss, apoptotic cells, specific parameter scores 0-4.
^{c} Inflammatory infiltrative phase: Neutr, other Polymorphs MNC mainly histiocyt & macrophages. BALT alv, interstial, alv-duct, bronchiole diffuse, patch cellular consol, specific parameter scores 0-4.

### Example 32

### D-galactose-Induced Accelerated Immunosenescence Aging Model as an Endogenous and Exogenous Assault Trigger Response

Systemic administration of D-galactose induces accelerated immune cell senescence affecting immune response at the time of challenge similar to aged mice. These phenomena are presumed to mimic the immune response profile of the elderly. The novel contemplated subject matter comprising polysaccharides and polyphenols (UP360 made in Example 9) was tested in this experimentally aged mouse model to demonstrate its immune-stimulating effects. Purpose-bred CD-1 mice (12 weeks old) were purchased and used for the accelerated aging study after 2 weeks of acclimation. Mice were randomly assigned to 4 immunized groups and 3 non-immunized groups. The immunized groups included G1=normal control+Vehicle (0.5% CMC), G2=D-galactose+vehicle, G3=D-galactose+UP360 400 mg/kg and G4=D-galactose+UP360 200 mg/kg. The non-immunized treatment groups included G1=normal control+Vehicle (0.5% CMC), G2=D-galactose+vehicle, and G3=D-galactose+UP360 400 mg/kg. While 10 animals were allocated in each treatment group for the immunized set, eight animals were included in each group for the non-immunized set.

Mice were injected with D-galactose at 500 mg/kg subcutaneously daily for 9 weeks to induce aging. On the 4^{th} week of induction, treatment with 2 doses of UP360 (200 mg/kg-Low dose and 400 mg/kg-High dose) suspended in 0.5% CMC orally commenced. One additional group of UP360 at 400 mg/kg was included to be used as a control for non-immunized mice. On the 7^{th} week, each mouse except those mice in non-immunized groups was injected with 3 μg of Fluarix quadrivalent IM (2020-2021 influenza season vaccine from GSK. It contained 60 μtg hemagglutinin—HA per 0.5 mL single human dose. The vaccine was formulated to contain 15 μg of each of 4 influenza strains such as H1N1, H3N2, B-Victoria lineage and B-Yamagata lineage) for immunization at a single dose.

Daily oral gavaging of UP360 comprising, and in some instances consisting of polysaccharides and polyphenols for the duration from the 4^{th} week to the 9^{th} week was carried out. At the time of necropsy, (i.e. 14-days after immunization), whole blood (1 mL) was collected and aliquoted—110 μL for flow cytometry immunity panel (delivered on ice to Flow Contract Site Laboratory, Bothell, Wash.), serum was isolated from the remaining blood (about 400 μL serum yield) for antibody ELISAs and enzymatic assays (Unigen, Tacoma Wash.), and 60 μL was shipped in two tubes for cytokine analysis via Fedex overnight to Sirona DX, Portland, Oreg. Weights of the thymus and spleen for each animal were measured to determine thymus and spleen indices. Representative images of the thymus and spleen were taken from each group. The spleens were kept on dry ice at the time of necropsy and transferred to −80° C. for future use. Paraformaldehyde and sucrose-fixed thymi were sent to Nationwide histology for Senescence-associated β-galactosidase staining and analysis.

### Example 33

### UP360 Produced a Statistically Significant Increase of Thymus Index

Repetitive subcutaneous administration of D-galactose into mice produces a poor immune response, resembling changes that occur in the normal aging process. the thymus is one of the most important immune organs that would be affected by chronic exposure to D-gal. The thymus index is a good indication of the strength of the immune function of the body. A higher thymus index corresponds to a stronger non-specific immune response. In the immunized mice, D-gal mice treated with the vehicle showed a significant reduction (54.5%) in the thymus index compared to the normal control mice. This reduction in thymus index was reversed by both dosages of UP360 comprising polysaccharides and polyphenols. Mice treated with UP360 orally at 400 mg/kg and 200 mg/kg showed a 52.9% and 50.6% increase in thymus index, respectively, when compared to the vehicle-treated D-gal group. This reversal was statistically significant compared to vehicle-treated D-gal mice for both doses of UP360. Similarly, the non-immunized mice treated with UP360 at 400 mg/kg also showed a statistically significant increase in the thymus index. This increase was found to be 26.9% when compared to the vehicle-treated D-gal mice. It was observed in this study that, regardless of immunization status, UP360 supplementation seemed to protect the mice from age-associated thymus involution.

TABLE 30
In vivo Treatment groups for Thymus protection

| | Thymus Index | | | |
|---|---|---|---|---|
| | Immunized | | Non-immunized | |
| Group | Mean ± Sd | P-value | Mean ± Sd | P-value |
| Normal Control + | 1.764 ± 0.389 | 0.00001 | 1.830 ± 0.535 | 0.00001 |
| Vehicle | | | | |
| D-Gal. 500 mg/kg + | 0.803 ± 0.279 | — | 0.980 ± 0.150 | — |
| Vehicle | | | | |
| D.gal + UP360 | 1.702 ± 0.347 | 0.00001 | 1.341 ± 0.200 | 0.002 |
| 400 mg/kg | | | | |
| D.gal + UP360 | 1.623 ± 0.297 | 0.00001 | — | — |
| 200 mg/kg | | | | |

### Example 34

### UP360 Supplementation Showed a Trend Toward Restoration of a Healthy Spleen Index

The spleen is the other significant organ in the immune system whereby its index is vital for healthy immune function. Injection of D-galactose at 500 mg/kg produced a statistically significant, 25.4%, reduction in the spleen index of immunized mice in our study. The non-immunized mice showed a 16.3% decrease in the spleen index.

A minimal to moderate increase in the spleen index was observed for mice treated with UP360 orally at 400 mg/kg in the immunized and non-immunized and 200 mg/kg in the immunized groups. While these improvements failed to reach statistical significance, UP360 treatment showed a trend toward inhibiting tissue atrophy, as evidenced by the increase of the spleen index.

TABLE 31
In vivo Treatment groups for spleen protection

| | Spleen Index | | | |
|---|---|---|---|---|
| | Immunized | | Non-immunized | |
| Group | Mean ± Sd | P-value | Mean ± Sd | P-value |
| Normal Control + | 3.473 ± 0.877 | 0.015 | 3.186 ± 0.726 | 0.104 |
| Vehicle | | | | |
| D-Gal. 500 mg/kg + | 2.586 ± 0.458 | — | 2.671 ± 0.292 | — |
| Vehicle | | | | |
| D.gal + UP360 | 2.624 ± 0.413 | 0.852 | 2.800 ± 0.488 | 0.560 |
| 400 mg/kg | | | | |
| D.gal + UP360 | 2.761 ± 0.399 | 0.399 | — | — |
| 200 mg/kg | | | | |

### Example 35

### UP360 Supplementation Protected Mice from Age-Associated Thymus Involution

At necropsy, the thymus was dissected from each mouse and fixed in prechilled paraformaldehyde for 24 hours before they were transferred to 30% sucrose solution for an additional 24 hours. Fixed tissues were then snap frozen in liquid nitrogen and shipped to

Nationwide histology packed in dry ice for analysis. Tissues were flash frozen in cryoprotectant and sectioned at 10-micron thickness onto Superfrost Plus slides. Tissues were then rinsed in PBS and the protocol for the β-galactosidase staining kit from Cell Signaling Technologies was followed. A light eosin counterstain was added for contrast and slides were mounted with non-aqueous mounting medium. The senescent cells were then counted in quadrants to determine the overall percentage of positive cells. An Olympus BH2, Nikon Eclipse 800 microscope with an Olympus DP26 camera operating with cellSens Standard 1.9 software were used for cell counting and imaging.

SA-β-gal staining detected senescent cells in each thymus to evaluate the immune organ protection effects of UP360 comprising polysaccharides and polyphenols. SA-β-Gal positive cells were found stained blue (expressing highly senescent-specific β-galactosidase) and randomly scattered throughout the cortex and medulla. The changes observed for the lower dose of UP360 thymus histology were in alignment with the thymus index data. As seen in Table 32, immunized mice treated with UP360 (200 mg/kg) showed a statistically significant reduction in the proportion of senescent cells when compared to the vehicle-treated D-gal mice. These findings further confirmed the immune cell and/or organ protection capacity of the novel composition UP360 comprising polysaccharides and polyphenols. Subcutaneous administration of D-gal produced a 157.8% increase in senescence cells when compared to the normal control mice, whereas, mice treated with UP360 at 200 mg/kg, showed a 42.7% reduction in senescence cells in comparison to that of the vehicle-treated D-gal mice.

TABLE 32
In vivo Treatment group for the changes of senescence cells

| | SA-β-gal Positive cells (%) | | |
|---|---|---|---|
| Group | Mean | SD | p-value |
| Control | 8.92 | 3.16 | 0.005 |
| D-gal control | 23.00 | 11.40 | — |
| D-gal + UP360 Low | 13.17 | 7.99 | 0.035 |

### Example 36

### Aloe-Based Composition UP360 Increased D-gal-induced Serum IgA

Serum was collected at the end of the study and assessed for markers of humoral immunity, including IgG. The immunized control group was not significantly different from the non-immunized control for IgA antibody level. The D-gal+200 mg/kg UP360 group had a trend toward being higher than the D-gal group (p=0.06), whereas the D-gal+400 mg/kg UP360 group had significantly higher serum IgA than the D-gal group.

TABLE 33
IgA antibodies in D-gal induced mouse sera treated with UP360

| IgA antibody | | p value | p value |
|---|---|---|---|
| (μg/mL serum) | Non-Immunized | vs Control | vs D-Gal |
| Control | 49 +/− 12 | — | — |
| D-Gal | 56 +/− 10 | 0.97 | — |
| D-Gal + 400 mg/kg UP360 | 67 +/− 54 | 0.16 | 0.16 |
| | | p value | p value |
| | Immunized | vs Control | vs D-Gal |
| Control | 48 +/− 20 | — | — |
| D-Gal | 39 +/− 15 | 0.50 | — |
| D-Gal + 200 mg/kg UP360 | 71 +/− 16 | 0.30 | 0.06 |
| D-Gal + 400 mg/kg UP360 | 91 +/− 16 | 0.21 | *0.03 |

*denotes statistical significance

### Example 37

### Effect of Aloe-Based Composition UP360 on CD45+ Cells (White Blood Cells)

Nine weeks after the study commenced, whole mouse blood was collected to assess the populations of white blood cells in general, and subpopulations of immune cells specifically. The data was analyzed using two methods, as percentages of cells that were positive for specific markers, and as cells per μL of blood (Alvarez DF) (Vera EJ). Because the D-gal-treated mice had high levels of CD45+ cells (white blood cells), the reporting of findings as percentages of CD45+ cells highlighted different findings than the reporting of findings as cells per μL of blood. Both data sets informed on the performance of UP360 as an immune booster.

TABLE 34
CD45+ cells white blood cells in whole mouse blood

| CD45+ Lymphocytes | | | |
|---|---|---|---|
| in whole blood | | | |
| (% of total cell | Non- | p value | p value |
| population) | immunized | vs Control | vs D-Gal |
| Control | 86 +/− 6.0 | — | — |
| D-Gal | 89 +/− 4.7 | 0.62 | — |
| D-Gal + 400 mg/kg UP360 | 90 +/− 2.2 | 0.36 | 0.65 |

| | | | | p value |
|---|---|---|---|---|
| | | p value | p value | vs Non- |
| | Immunized | vs Control | vs D-Gal | immunized |
| Control | 72 +/− 7.3 | — | — | *0.04 |
| D-Gal | 92 +/− 3.5 | *0.002 | — | 0.45 |
| D-Gal + 200 | 77 +/− 6.1 | 0.48 | *0.004 | N/A |
| mg/kg UP360 | | | | |
| D-Gal + 400 | 85 +/− 4.4 | *0.03 | 0.08 | 0.13 |
| mg/kg UP360 | | | | |

*denotes statistical significance

After red blood cells were removed from whole blood, 7-amino-actinomycin D was used to distinguish live and dead cells and CD45 was used to mark white blood cells. Table 34 shows the amount of CD45+ cells (white blood cells) among the live cell population from each group. The immunized control group had a significant decrease in the percentage of white blood cells compared to the non-immunized control group, potentially demonstrating expansion of other cell types upon influenza vaccination. Compared to the immunized control group, the D-gal group had a significantly higher percentage of white blood cells in the blood per live cell population, which was decreased to the level of the control in the 200 mg/kg UP360+D-gal group (UP360 Low).

### Example 38

### Effect of Aloe-Based Composition on CD3+ T-Cells in Whole Blood (% of Lymphocyte Population)

CD3+CD45+ cells are the T cell population. Expressed as a percentage of all white blood cells (CD45+ cells), we found that two weeks after influenza vaccination, there was a trend toward a decrease in circulating T cells in the immunized control animals compared to the non-immunized control (p=0.07). The immunized animals treated with 400 mg/kg UP360+D-gal had a significantly higher percentage of circulating T cells than the D-gal group, indicating that UP360 comprising polysaccharides and polyphenols increased CD3+ T cell expansion or differentiation in response to the influenza vaccination.

TABLE 35
CD3+ T cells in whole mouse blood

| CD3+ T-cells in | | | |
|---|---|---|---|
| whole blood | | | |
| (% of lymphocyte | Non- | p value | p value |
| population) | immunized | vs Control | vs D-Gal |
| Control | 26 +/− 5.9 | — | — |
| D-Gal | 19 +/− 2.7 | 0.13 | — |
| D-Gal + 400 mg/kg UP360 | 21 +/− 0.8 | 0.25 | 0.28 |

| | | | | p value |
|---|---|---|---|---|
| | | p value | p value | vs Non- |
| | Immunized | vs Control | vs D-Gal | Immunized |
| Control | 17 +/− 2.3 | — | — | 0.07 |
| D-Gal | 16 +/− 2.4 | 0.48 | — | 0.23 |
| D-Gal + 200 | 19 +/− 2.2 | 0.42 | 0.14 | N/A |
| mg/kg UP360 | | | | |
| D-Gal + 400 | 22 +/− 2.2 | *0.048 | *0.01 | 0.81 |
| mg/kg UP360 | | | | |

*denotes statistical significance

### Example 39

### Effect of Aloe-Based Composition on CD4+ Helper T Cells in Whole Blood (% of Lymphocyte Population)

CD45+CD3+CD4+ cells are Helper T cells, the cells that recognize antigens on antigen-presenting cells and respond with cell division and cytokine secretion. Expressed as a percentage of all white blood cells (CD45+ cells), we found that two weeks after influenza vaccination, the immunized animals treated with 200 and 400 mg/kg UP360+D-gal had a significantly higher percentage of circulating Helper T cells than the D-gal group, indicating that UP360 comprising polysaccharides and polyphenols increased Helper T cell expansion or differentiation in response to influenza vaccination.

TABLE 36
CD3+CD4+ Helper T cells in whole mouse blood.

| CD4+ Helper T cells | | | |
|---|---|---|---|
| in whole blood | | | |
| (% of lymphocyte | Non- | p value | p value |
| population) | immunized | vs Control | vs D-Gal |
| Control | 17 +/− 4.1 | — | — |
| D-Gal | 13 +/− 1.8 | 0.25 | — |
| D-Gal + 400 mg/kg UP360 | 14 +/− 0.6 | 0.40 | 0.37 |

| | | | | p value |
|---|---|---|---|---|
| | | p value | p value | vs Non- |
| | Immunized | vs Control | vs D-Gal | Immunized |
| Control | 11 +/− 1.9 | — | — | 0.12 |
| D-Gal | 10 +/− 1.7 | 0.37 | — | 0.11 |
| D-Gal + 200 | 12 +/− 1.7 | 0.59 | 0.14 | N/A |
| mg/kg UP360 | | | | |
| D-Gal + 400 | 14 +/− 1.4 | 0.09 | *0.009 | 0.95 |
| mg/kg UP360 | | | | |

*denotes statistical significance

### Example 40

### Effect of Aloe-Based Composition on CD8+ Cytotoxic T Cells in Whole Blood (% of Lymphocyte Population)

CD45+CD3+CD8+ cells are Cytotoxic T cells, the cells that respond to immune challenges with cell division and secretion of apoptosis-promoting enzymes to kill infected cells. Expressed as a percentage of all white blood cells (CD45+ cells), we found that two weeks after influenza vaccination, the immunized animals treated with 200 and 400 mg/kg UP360+D-Gal had a trend toward a higher percentage of circulating Cytotoxic T cells than the D-gal group, and significantly lower percentages of circulating Cytotoxic T cells in the immunized control group and the non-immunized D-gal group than the non-immunized control group.

TABLE 37
CD3+CD8+ Cytotoxic T cells in whole mouse blood.

| CD8+ Cytotoxic T cells | | | |
|---|---|---|---|
| in whole blood | | | |
| (% of lymphocyte | Non- | p value | p value |
| population) | immunized | vs Control | vs D-Gal |
| Control | 8.6 +/− 2.0 | — | — |
| D-Gal | 5.1 +/− 0.8 | *0.049 | — |
| D-Gal + 400 mg/kg UP360 | 6.1 +/− 0.6 | 0.12 | 0.22 |

| | | | | p value |
|---|---|---|---|---|
| | | p value | p value | vs Non- |
| | Immunized | vs Control | vs D-Gal | immunized |
| Control | 4.7 +/− 0.6 | — | — | *0.03 |
| D-Gal | 4.9 +/− 1.1 | 0.82 | — | 0.82 |
| D-Gal + 200 | 5.7 +/− 0.7 | 0.12 | 0.35 | N/A |
| mg/kg UP360 | | | | |
| D-Gal + 400 | 6.4 +/− 0.9 | *0.04 | 0.13 | 0.68 |
| mg/kg UP360 | | | | |

*denotes statistical significance

### Example 41

### Effect of Aloe-Based Composition on NKp46+ Natural Killer Cells in Whole Blood (% of Lymphocyte Population)

We utilized two different Natural Killer cell markers, mouse CD49b and NKp46, to identify the percentage of Natural Killer cells in the white blood cell population. Natural Killer cells are involved in the innate immune system. When activated, they secrete cytokines and granules to recruit immune cells and directly cause cell death in cells infected with pathogens, thus they are important for immediate immune responses to pathogens and are active early in systemic infections. CD49b is an integrin that is present specifically on most Natural Killer cells and also a subset of T cells that may be Natural Killer T (NKT) cells. NKp46 is a Natural Cytotoxicity Receptor that is exclusively present on Natural Killer cells and does not mark NKT cells. NKTs and NK-like T cells are also excluded based on their expression of CD3, since NKs are generally CD45+CD3−CD49b+NKp46+ (Goh W) (Narni-Mancinelli E). Expressed as a percentage of all white blood cells (CD45+ cells), we found that two weeks after influenza vaccination, there were no significant differences in the CD3−CD49b+ population among any of the groups. When we looked at the CD3-NKp46+ populations, however, the immunized animals treated with D-gal had a significantly lower percentage of Natural Killer cells than the immunized control group, and both immunized 200 and 400 mg/kg UP360+D-gal-treated groups had significantly higher percentages of circulating Natural Killer cells than the immunized D-gal group. The non-immunized 400 mg/kg UP360+D-gal group also had a significantly higher percentage of circulating Natural Killer cells than the non-immunized D-gal group.

TABLE 38
CD3-NKp46+ Natural Killer cells in whole mouse blood.

| NKp46+ Natural Killer | | | |
|---|---|---|---|
| cells in whole blood | | | |
| (% of lymphocyte | Non- | p value | p value |
| population) | immunized | vs Control | vsD-Gal |
| Control | 41 +/− 6.0 | — | — |
| D-Gal | 38 +/− 4.2 | 0.68 | — |
| D-Gal + 400 mg/kg UP360 | 51 +/− 6.0 | 0.11 | *0.03 |

| | | | | p value |
|---|---|---|---|---|
| | | p value | p value | vs Non- |
| | Immunized | vs Control | vs D-Gal | Immunized |
| Control | 45 +/− 8.4 | — | — | 0.51 |
| D-Gal | 27 +/− 7.8 | *0.02 | — | 0.06 |
| D-Gal + 200 | 43 +/− 5.7 | 0.70 | *0.02 | N/A |
| mg/kg UP360 | | | | |
| D-Gal + 400 | 42 +/− 3.0 | 0.65 | *0.01 | 0.11 |
| mg/kg UP360 | | | | |

*denotes statistical significance

These results were confounding because the two Natural Killer cell markers gave drastically different results. Natural Killer cell markers vary greatly depending on mouse strain. Because NKp46 is a marker that is very specific to Natural Killer cells in most mouse strains, and CD49b can mark other cell types, NKp46 may be more reliable. The percentages of NK cells among the CD45+ cells are high for NKp46, however, compared to CD49b, which aligns more closely to human NK numbers in peripheral blood (Angelo LS). NK cells in peripheral mouse blood may be similar to human, or they may be as high as was detected for NKp46.

### Example 42

### Effect of Aloe-Based Composition on TCRγδ+ Gamma Delta T Cells in Whole Blood (% of Lymphocyte Population)

CD45+CD3+TCRγδ+ cells are Gamma delta T cells, a small population of T cells that may have diverse activities and affect both the innate and adaptive immune responses. They are localized to mucosa to elicit a first line of defense against pathogens and aid in mounting adaptive immune responses.

TABLE 39
CD3+TCRγδ+ Gamma delta T cells in whole mouse blood

| TCRγδ+ Gamma delta T cells | | | |
|---|---|---|---|
| in whole blood | | | |
| (% of lymphocyte | Non- | p value | p value |
| population) | immunized | vs Control | vs D-Gal |
| Control | 0.39 +/− 0.08 | — | — |
| D-Gal | 0.26 +/− 0.05 | 0.09 | — |
| D-Gal + 400 mg/kg UP360 | 0.33 +/− 0.05 | 0.38 | 0.22 |

| | | | | p value |
|---|---|---|---|---|
| | | p value | p value | vs Non- |
| | Immunized | vs Control | vs D-Gal | immunized |
| Control | 0.32 +/− 0.07 | — | — | 0.34 |
| D-Gal | 0.28 +/− 0.05 | 0.48 | — | 0.69 |
| D-Gal + 200 | 0.39 +/− 0.06 | 0.25 | *0.03 | N/A |
| mg/kg UP360 | | | | |

*denotes statistical significance

Expressed as a percentage of all T cells (CD3+ cells), we found that two weeks after influenza vaccination, the immunized animals treated with 200 mg/kg UP360+D-gal had a significantly higher percentage of circulating Gamma delta T cells than the D-gal group, and the 400 mg/kg UP360+D-gal group had a trend toward a higher percentage of circulating Gamma delta T cells than the D-gal. This may have indicated that the UP360 comprising polysaccharides and polyphenols treated groups are better equipped to mount immune responses to pathogens encountered in mucous membranes.

### Example 43

### Effect of Aloe-Based Composition on CD45+ Lymphocytes in Whole Blood (Cells/μL)

We also analyzed the cell populations in whole blood from non-immunized and influenza-vaccinated mouse groups as cells per μL of whole blood. These data represent the immune cell populations without considering the differences in CD45+ cells and CD3+ cells that could confound the data represented per percentage of cells that are positive for those markers. Generally, we found that the significant differences obtained from analyzing the data in this manner pertained to the non-immunized mouse groups instead of the immunized groups.

TABLE 40
CD45+ white blood cells in whole mouse blood.

| CD45+ Lymphocytes | | | |
|---|---|---|---|
| in whole blood | Non- | p value | p value |
| (cells/μL) | immunized | vs Control | vs D-Gal |
| Control | 3462 +/− 941 | — | — |
| D-Gal | 5778 +/− 764 | 0.53 | — |
| D-Gal + 400 mg/kg UP360 | 8025 +/− 1673 | *0.006 | 0.12 |

| | | | | p value |
|---|---|---|---|---|
| | | p value | p value | vs Non- |
| | Immunized | vs Control | vs D-Gal | Immunized |
| Control | 5040 +/− 1622 | — | — | 0.22 |
| D-Gal | 5473 +/− 1214 | 0.74 | — | 0.75 |
| D-Gal + 200 | 5059 +/− 781 | 0.99 | 0.66 | N/A |
| mg/kg UP360 | | | | |
| D-Gal + 400 | 4281 +/− 582 | 0.50 | 0.18 | *0.02 |
| mg/kg UP360 | | | | |

*denotes statistical significance

CD45+ cells per μL of blood were not significantly different among the non-immunized and immunized mouse groups, but there was a higher number of CD45+ cells in the non-immunized 400 mg/kg UP360+D-gal group than the non-immunized D-gal alone.

### Example 44

### Effect of Aloe-Based Composition on CD3+ T Cells, CD4+ Helper T and CD8+ Cytotoxic T Cells in Whole Blood (Cells/μL)

Compared to the D-gal non-immunized group, the non-immunized 400 mg/kg UP360+D-gal group had significantly higher CD3+ cells per μL of whole blood. The same increases were seen for CD3+CD4+ Helper T cells and CD3+CD8+ Cytotoxic T cells. These findings indicated higher levels of Helper T cells, Cytotoxic T cells, and T cells in general in the UP360+D-gal non-immunized group compared to the D-gal only group, which may have indicated better immune surveillance and “readiness” in the UP360-treated group.

TABLE 41
CD3+ T cells in whole mouse blood

| CD3+ T cells | | | |
|---|---|---|---|
| in whole blood | | p value | p value |
| (cells/μL) | Non-Immunized | vs Control | vs D-Gal |
| Control | 921 +/− 401 | — | — |
| D-Gal | 1053 +/− 156 | 0.68 | — |
| D-Gal + 400 mg/kg UP360 | 1656 +/− 281 | 0.054 | *0.02 |

*denotes statistical significance

TABLE 42
CD3+CD4+ Helper T cells in whole mouse blood

| CD4+ Helper T cells | | | |
|---|---|---|---|
| in whole blood | Non- | p value | p value |
| (cells/μL) | immunized | vs Control | vs D-Gal |
| Control | 576 +/− 245 | — | — |
| D-Gal | 722 +/− 112 | 0.46 | — |
| D-Gal + 400 mg/kg UP360 | 1108 +/− 184 | *0.03 | *0.03 |

*denotes statistical significance

TABLE 43
CD3+CD8+ Cytotoxic T cells in whole mouse blood

| CD8+ Cytotoxic T cells | | | |
|---|---|---|---|
| in whole blood | Non- | p value | p value |
| (cells/μL) | immunized | vs Control | vs D-Gal |
| Control | 309 +/− 150 | — | — |
| D-Gal | 283 +/− 45 | 0.82 | — |
| D-Gal + 400 mg/kg UP360 | 474 +/− 101 | 0.22 | *0.03 |

*denotes statistical significance

### Example 45

### Effect of Aloe-Based Composition on CD49b+Natural Killer Cells in Whole Blood (Cells/μL)

The two markers used to detect Natural Killer cells gave similar results, with a trend toward an increase in CD49b+ NK cells and a significant increase in the NKp46+ NK cells in the non-immunized 400 mg/kg U360+D-gal group compared to the non-immunized D-gal alone. The cell counts for each marker varied drastically, similar to the variation in the percentages of CD45+ cells as analyzed previously. Both markers indicated an enrichment of NK cells in the non-immunized UP360+D-gal group compared to the non-immunized D-gal alone, which may have been another indication of immune surveillance and immune “readiness” in the UP360-treated group.

TABLE 44
CD3-CD49b+ Natural Killer cells in whole mouse blood

| CD49b+ Natural Killer cells | Non- | p value | p value |
|---|---|---|---|
| in whole blood (cells/μL) | immunized | vs Control | vs D-Gal |
| Control | 516 +/− 156 | — | — |
| D-Gal | 939 +/− 152 | *0.02 | — |
| D-Gal + 400 mg/kg UP360 | 1323 +/− 355 | *0.02 | 0.19 |

*denotes statistical significance

TABLE 45
CD3-NKp46+ Natural Killer cells in whole mouse blood

| NKp46+ Natural Killer cells | Non- | p value | p value |
|---|---|---|---|
| in whole blood (cells/μL) | immunized | vs Control | vs D-Gal |
| Control | 1457 +/− 433 | — | — |
| D-Gal | 2221 +/− 388 | 0.08 | — |
| D-Gal + 400 mg/kg UP360 | 4233 +/− 1092 | *0.008 | *0.04 |

*denotes statistical significance

### Example 46

### Effect of Aloe-Based Composition on Ly6C+ Granulocytes in Whole Blood (Cells/μL)

The CD3-Ly6C+ granulocytes per μL were not significantly different among treatment groups and the D-gal group. There were significantly increased granulocytes in the non-immunized D-gal group and non-immunized 400 mg/kg UP360+D-gal group compared to the non-immunized control group, and a trend toward reduced granulocytes per μL in the immunized UP360+D-gal groups compared to the immunized D-gal and control. There was a statistically significant decrease in granulocytes in the immunized 400 mg/kg UP360+D-gal group compared to the non-immunized400 mg/kg UP360+D-gal group.

TABLE 46
CD3-Ly6C+ Granulocytes in whole mouse blood

| Ly6C+ Granulocytes | | p value | p value |
|---|---|---|---|
| in whole blood (cells/μL) | Non-Immunized | vs Control | vs D-Gal |
| Control | 495 +/− 168 | — | — |
| D-Gal | 1774 +/− 600 | *0.02 | — |
| D-Gal + 400 mg/kg UP360 | 1721 +/− 363 | *0.002 | 0.92 |

| | | | | p value |
|---|---|---|---|---|
| | | p value | p value | vs Non- |
| | Immunized | vs Control | vs D-Gal | Immunized |
| Control | 1622 +/− 968 | — | — | 0.10 |
| D-Gal | 1725 +/− 717 | 0.89 | — | 0.94 |
| D-Gal + 400 | 861 +/− 133 | 0.25 | 0.09 | *0.01 |
| mg/kg UP360 | | | | |

*denotes statistical significance

### Example 47

### Effect of Aloe-Based Composition on B220+ B Cells in Whole Blood (Cells/μL)

There were no significant differences in CD3-B220+ B cells among the treatment groups as represented by cell per μL of whole blood, but there was a trend toward increased B cells in the non-immunized 400 mg/kg UP360+D-gal group compared to the non-immunized D-gal alone.

TABLE 47
CD3-B220+ B cells in whole mouse blood

| B220+ B cells in whole | | p value | p value |
|---|---|---|---|
| blood (cells/μL) | Non-Immunized | vs Control | vs D-Gal |
| Control | 1649 +/− 446 | — | — |
| D-Gal | 2365 +/− 553 | 0.18 | — |
| D-Gal + 400 mg/kg UP360 | 3889 +/− 1130 | *0.03 | 0.12 |

*denotes statistical significance

### Example 48

### Effect of Aloe-Based Composition on TCRγδ+ Gamma Delta T Cells, CD4+TCRγδ+ Gamma Delta Helper T Cells, CD8+TCRγδ+ Gamma Delta Cytotoxic T Cells in Whole Blood (Cells/μL)

CD3+TCRγδ+ Gamma delta T cells per μL whole blood were increased in the non-immunized 400 mg/kg UP360+D-gal group compared to the non-immunized D-gal alone. This was also found for the CD3+CD4+TCRγδ+ Helper Gamma delta T cell population. There were no significant differences in the CD3+CD8+TCRγδ+ Cytotoxic Gamma delta T cell population. These findings indicated increase immune surveillance and immune “readiness” in the T cell populations of the mucosa.

TABLE 48
CD3+TCRγδ+ Gamma delta T cells in whole mouse blood

| TCRγδ+ Gamma delta T cells | Non- | p value | p value |
|---|---|---|---|
| in whole blood (cells/μL) | immunized | vs Control | vs D-Gal |
| Control | 13 +/− 4.2 | — | — |
| D-Gal | 14 +/− 2.3 | 0.67 | — |
| D-Gal + 400 mg/kg UP360 | 25 +/− 5.2 | *0.02 | *0.02 |

*denotes statistical significance

TABLE 49
CD3+CD4+TCRγδ+ Gamma delta Helper
T cells in whole mouse blood

| CD4+TCRγδ+ Gamma | | | |
|---|---|---|---|
| delta Helper T cells | Non- | p value | p value |
| in whole blood (cells/μL) | immunized | vs Control | vs D-Gal |
| Control | 11 +/− 3.3 | — | — |
| D-Gal | 12 +/− 2.4 | 0.51 | — |
| D-Gal + 400 mg/kg UP360 | 21 +/− 4.2 | *0.02 | *0.04 |

*denotes statistical significance

TABLE 50
_CD8+TCRγδ+ Gamma delta Cytotoxic T
cells in whole blood (cells/μL)

| CD8+TCRγδ+ Gamma | | | |
|---|---|---|---|
| delta Cytotoxic T cells | Non- | p value | p value |
| in whole blood (cells/μL) | immunized | vs Control | vs D-Gal |
| Control | 1.1 +/− 0.24 | — | — |
| D-Gal | 1.9 +/− 0.55 | 0.09 | — |
| D-Gal + 400 mg/kg UP360 | 2.3 +/− 0.78 | 0.59 | 0.55 |

| | | | | p value |
|---|---|---|---|---|
| | | p value | p value | vs Non- |
| | Immunized | vs Control | vs D-Gal | Immunized |
| Control | 1.3 +/− 0.39 | — | — | 0.51 |
| D-Gal | 2.1 +/− 0.71 | 0.15 | — | 0.76 |
| D-Gal + 200 | 2.7 +/− 0.86 | *0.04 | 0.43 | N/A |
| mg/kg UP360 | | | | |

*denotes statistical significance

### Example 49

### Aloe-Based Composition Increased Superoxide Dismutase (SOD) Significantly

The mechanism by which D-gal causes an aging phenotype is through the generation of free radicals, especially Advanced Glycation End Products. We sought to measure antioxidation enzyme concentration and free radical levels to determine whether UP360 affected this aspect of the mouse model (Azman KF).

TABLE 51
Superoxide dismutase content of mouse serum samples

| Superoxide dismutase (SOD) | Non- | p value vs | p value vs |
|---|---|---|---|
| in serum (U/mL) | Immunized | Control | D-Gal |
| Control | 11.9 +/− 1.0 | — | — |
| D-Gal | 9.6 +/− 1.1 | *0.04 | — |
| D-Gal + 400 mg/kg UP360 | 10.8 +/− 1.3 | 0.34 | 0.32 |

| | | | | p value |
|---|---|---|---|---|
| | | p value | p value | vs Non- |
| | Immunized | vs Control | vs D-Gal | Immunized |
| Control | 7.7 +/− 1.2 | — | — | *0.0005 |
| D-Gal | 7.2 +/− 1.2 | 0.66 | — | *0.02 |
| D-Gal + 200 | 10.1 +/− 0.9 | *0.01 | *0.003 | N/A |
| mg/kg UP360 | | | | |
| D-Gal + 400 | 9.2 +/− 0.7 | *0.05 | *0.01 | 0.17 |
| mg/kg UP360 | | | | |

A Unit of SOD is the amount required to exhibit 50% dismutation of the superoxide radical.
*denotes statistical significance

Superoxide dismutase neutralizes oxygen radicals to prevent oxidative damage to cellular structures, proteins, and nucleic acids. Reactive oxygen species are used as secondary messengers for immune signaling (Ighodaro OM). Increased expression of antioxidation enzymes is indicative of the capability to neutralize excess reactive oxygen species. We tested immunized mouse serum samples for superoxide dismutase enzyme levels and found that the UP360+D-Gal groups had significantly higher levels of superoxide dismutase than the D-Gal group.

### Example 50

### Effect of Aloe-Based Composition on Protein Expression of Nrf2

Nrf2 is a transcription factor that is activated in oxidative stress conditions and upregulates involved in the antioxidant response. Prolonged immune system activation or oxidative stress causes upregulation of Nrf2. Spleen homogenates were run on SDS-PAGE, transferred, and blotted for the proteins mentioned. Band intensity was measured by densitometry and normalized for each protein of interest to the β-actin loading control. Semi-quantitation of each protein of interest was compared for each group and was found that the immunized 200 mg/kg UP360+D-gal and 400 mg/kg UP360+D-gal groups had significantly higher Nrf2 than the D-gal alone, indicating an increase in antioxidation pathway activation in the UP360 groups.

TABLE 52
Nrf2 protein levels of immunized mouse spleen homogenates
normalized to β-actin and relative to the control group

| Nrf2 protein expression | | | |
|---|---|---|---|
| normalized to β- | | | |
| actin and relative | Non- | p value | p value |
| to the Control | Immunized | vs Control | vs D-Gal |
| Control | 1.0 +/− 0.14 | — | — |
| D-Gal | 1.0 +/− 0.23 | 0.99 | — |
| D-Gal + 400 mg/kg UP360 | 0.7 +/− 0.11 | *0.03 | 0.12 |

| | | | | p value |
|---|---|---|---|---|
| | | p value | p value | vs Non- |
| | Immunized | vs Control | vs D-Gal | Immunized |
| Control | 1.0 +/− 0.23 | — | — | *0.0002 |
| D-Gal | 1.2 +/− 0.43 | 0.58 | — | *0.003 |
| D-Gal + 200 | 2.5 +/− 0.57 | *0.002 | *0.01 | N/A |
| mg/kg UP360 | | | | |
| D-Gal + 400 | 4.9 +/− 1.55 | *0.003 | *0.004 | *0.01 |
| mg/kg UP360 | | | | |

*denotes statistical significance

### Example 51

### Effect of Aloe-Based Composition (UP360) on Mitigating Oxidative Stress Plus Pulmonary Infection Induced Mice Mortality Rate and Acute Inflammatory Lung Injury

Effect of contemplated Aloe-based compositions, including UP360, comprising and, in some embodiments, consisting of polysaccharides and polyphenols made in Example 9 on mortality was evaluated using Hyperoxia and microbial (Pseudomonas aeruginosa (PA)) infection induced mice.

Mice were acclimated for a week before induction. To investigate whether UP360 can reduce animal mortality and increase their survival, mice were exposed to hyperoxia (>90% oxygen for 72 hours) following a treatment with UP360 for seven days and continued for these 3 days before being inoculated with the PA. Mice were observed for 48 hours after bacteria inoculation. Pre-exposure to hyperoxia caused a significantly higher mortality (02), compared to the mice remained at room air (RA, Table 53). Intestinally, we found unexpectedly that a substantial mortality 24-hour post PA inoculation in mice exposed to hyperoxia for 48 hours only prior to PA inoculation. Compared to the 9% mortality in mice remained in room air (RA) and received the same amount of PA, 64% mortality was observed in mice treated with hyperoxia for 2 days prior to PA inoculation. On the other hand, mice treated with prophylactically with resveratrol (RES) and UP360 comprising polysaccharides and polyphenols, for 7 days prior to exposure to hyperoxia for 2 days and PA inoculated afterwards had mortality rate of 27%, and 31%, respectively, 24 hours post-inoculation. These results suggest that UP360 offers an improved advantage in reducing animal mortality. These survival data observed for contemplated Aloe-based compositions, including UP360, comprising and, in some embodiments, consisting of polysaccharides and polyphenols are in agreement with the data documented on LPS induced survival studies on Examples 20-22 where UP360 supplementation produced statistically significant reduction in mortality of animals.

TABLE 53
The effects of UP360 on hyperoxia-
induced mortality in PA-infected mice

| | | | RES | UP360 |
|---|---|---|---|---|
| | RA | O2 | (50 mg/kg) | (500 mg/kg) |
| Dead animals | 1 | 9 | 3 | 4 |
| Total animals | 11 | 14 | 11 | 13 |
| Mortality % | 9.09% | 64.29% | 27.27% | 30.77% |

Following confirmation of beneficial effect of the Aloe-based composition in reducing the mortality rate of hyperoxia and PA induced animals, oxidative stress-exacerbated acute lung injury-induced by bacterial infection was conducted. Mice were acclimated for a week. Treated animals by oral administration with UP360 (500 mg/kg) and Resveratrol (50 mg/kg) for 7 days before hyperoxia exposure. Exposed mice to >99% O₂ for 48 hours while maintaining the daily test material oral administration. Inoculated mice with PA (5×10⁸ CFUs) via intranasal aspiration, still maintain the daily treatment of test materials. Returned mice to 21% O₂ after inoculation. Harvested Bronchoalveolar lavage (BAL), collected blood samples and lung tissues 24 hours after infection. Measured total protein content in BAL and determined the numbers of viable bacteria in the BAL and lungs. Run assays to determine biomarkers listed in the Table 54 such as TNF-α, IL-1, IL-6, CRP, IL-8, IL-10, HMGB-1, MPO, MIP-2, NF-kappaB, Nrf2, macrophage count, neutrophil count, disease severity in histology.

As seen in Table 54, Aloe based composition showed statistically significant effect on bacterial clearance in the airways in hyperoxic and microbial infected mice. Previously, it has been shown that exposure to hyperoxia can compromise host defense against bacterial infections, resulting in higher bacterial loads in the airways (Patel et al., 2013). Results in Table 54 indicate bacterial load in the airways is elevated significantly by preexposure of the mice to hyperoxia (O2), compared to that of mice remained at room air (RA). Corresponding to the significantly reduce lung injury in mice treated with resveratrol, the airway bacterial load was significantly lower in these mice (RES).

TABLE 54
Aloe based composition showed statistically significant
effect on bacterial clearance in the airways in
hyperoxic and microbial infected mice.

| | Dosage | | ×10⁵ CFU/mL | P-values |
|---|---|---|---|---|
| Group | (mg/kg) | N | (Mean ± SE) | vs O2 |
| RA | 0 | 8 | 71.7 ± 67.2 | 0.0128 |
| O2 | 0 | 7 | 2592.7 ± 1316.1 | — |
| RES | 50 | 5 | 2.4 ± 0.7 | 0.0259 |
| UP360 | 500 | 8 | 505.9 ± 174.2 | 0.0195 |

Statistical analysis: Dunnett's multiple comparisons test

TABLE 55
Assay priority orders for biomarkers
from BAL, Serum and lung homogenate

| Priority | Sample | Biomarker |
|---|---|---|
| 1 | BAL | Leukocytes, HMGB1, TNF-α, IL-1, IL-6 |
| | homogenate | MPO, NFκB, HMGB1, (may be Nrf2) |
| 2 | BAL | MIP-2 |
| | Serum | HMGB1, TNF-α, IL-1, IL-6, CRP, Il-8, IL-10 |

Similarly, mice treated with UP360 had a significantly lower amount of bacteria load in their airways, compared to mice exposed to hyperoxia and treated with vehicle alone. The difference of the bacterial load in airways was statistically significant compared to that of mice treated with hyperoxia and vehicle control (O2). These results suggest that UP360 can in fact reduce bacterial load in Airways.

### Example 52

### Evaluation of Aloe-Based Compositions Comprising Polysaccharides and Polyphenols in Human Clinical Trial

Protocol: A randomized, triple-blind, placebo-controlled, parallel clinical trial to investigate a product on supporting immune function in healthy adults. The objective of this study was to investigate the efficacy of the investigational product (IP), UP360 comprising, and in some instances consisting of polysaccharides and polyphenols, made in Example 9, on supporting immune function in healthy adults.

In a randomized, triple-blind, placebo-controlled, parallel study the efficacy of the investigational product on supporting immune function in a healthy adult population in the 28 days before and 28 days after vaccination was evaluated. The study included males and females between 40 and 80 years of age, inclusive, who had not yet, but were willing, to receive the influenza vaccine, agreed to provide a verbal history of flu vaccination, agreed to maintain current lifestyle habits as much as possible throughout the study depending on their ability to maintain the following: diet, medications, supplements, exercise, and sleep and avoid taking new supplements, healthy, as determined by medical history and laboratory results, as assessed by Qualified Investigator (QI), willing to complete questionnaires and diaries associated with the study and to complete all clinic visits, and provided voluntary, written, informed consent to participate in the study.

Excluded were the following subjects: 1. Women who were pregnant, breast feeding, or planning to become pregnant during the study. 2. Participants with a known allergy to the active or inactive ingredients in UP360, placebo, or influenza vaccine. 3. Unvaccinated participants with flu prior to baseline from September 2020 or prior to Day 28 vaccination. 4. Participants self-reporting a diagnosis of COVID-19 prior to baseline or prior to Day 28 vaccination. 5. Participants who received the COVID-19 vaccine. 6. Current use of prescribed immunomodulators (including corticosteroids), such as immunosuppressants or immunostimulants, within 4 weeks of baseline. 7. Current use of dietary supplement or herbal medicines associated with boosting or modulating the immune system, unless willing to washout.

| Study Arm | Number of Participants |
|---|---|
| UP360 + Flu Vaccine | N = 25 |
| Placebo + Flu Vaccine | N = 25 |
| Total | N = 50 |

The study subjects were expected to participate in the study for up to a maximum of 56 days. Subjects attended the study at Visit 1 (Screening, Day-45 to -4) for informed consent and at Visit 2 (Baseline, Day 0) for confirmation of eligibility and randomization.

The primary and secondary efficacy and safety endpoints for the study were assessed at Visits 2 (Day 0), Visit 3 (Day 28), and Visit 4 (Day 56). Demographic information and medical history were recorded at the screening visit. Study subjects took UP360 daily leading up to an influenza vaccination, (at Day 28), then continued taking daily UP360 for an addition 4 weeks (up to Day 56).

The primary study outcomes were the difference between UP360 and placebo in the changes in immune parameters as assessed by lymphocyte populations (CD3+, CD4+, CD8+, CD45+, TCRγδ+, CD3−CD16+56+) and immunoglobulins (IgG, IgM, and IgA) in blood from baseline at Day 28 and 56.

Statistical analysis was carried out and summary statistics including means, medians, standard deviations, minimums, maximums, proportions (if categorical) on demographic characteristics and outcome measures were obtained for the overall sample and by study groups. Analysis of Variance (ANOVA) was used to examine differences in the averages of continuous variables between the two treatment groups (UP360, and placebo) when normality assumption was satisfied, and Kruskal-Wallis test was used when normality assumption was not satisfied. Chi-square and Fisher exact tests (when cells have counts less than 5) as appropriate were used to investigate differences for categorical variables. Repeated measures analysis of variance (Linear Mixed Model) was used to examine differences in the average values of outcomes over time between the treatment groups. Baseline value was included as a covariate in each model. Repeated measures analysis of variance (Linear Mixed Model) was also used to examine differences in the average values of changes of outcomes over time (from baseline at 28 days, at 56 days and from day 28 at day 56) between the two treatment groups, baseline value was included as a covariate in each model. Pairwise statistical significance from LMM (between groups and within group). Bonferroni adjustment was used for the pairwise comparisons. Statistical significance is defined as p-values<0.05. Statistical Analysis System software version 9.4 (SAS Institute Inc., Cary, N.C., USA) was used to perform the analysis.

Statistically significant outcome was observed for the primary end points (TCRγδ+ and CD45+ cells) in the preliminary clinical data report. As seen in table 56, subjects who received contemplated Aloe-based compositions, including UP360, comprising and, in some embodiments, consisting of polysaccharides and polyphenols showed statistically significant increase in the gamma delta T-cell percent cell population at multiple time points in comparison to those received the placebo. While subjects in the placebo group showed a 10.5 and 5.6% reduction in the percent of TCRγδ+ cells, contemplated Aloe-based compositions, including UP360, comprising and, in some embodiments, consisting of polysaccharides and polyphenols showed 21.5% and 24.5% increase in the percent of TCRγδ+ cells populations at days 28 and 56 post administration, respectively. Compared to Placebo, subjects who received the Aloe-based composition showed 23.5% and 38.9% (P<0.001) increase in the percent of TCRγδ+ cells populations at days 28 and 56 post administration of treatment, respectively. These increase in the percent of TCRγδ+ cells populations changes observed at day 0 to 56 (p=0.0002) and day 28 to 56 (p<0.0108) were statistically significant for the Aloe-based composition (UP360) made in Example 9 in comparison to the placebo. Similarly, these changes for the same time frame were statistically significant for the Aloe-based

TABLE 56
The changes of % of TCRγδ+ cells in UP360 vs Placebo

| | UP360 | Placebo | | |
|---|---|---|---|---|
| | (% of cell | (% of cell | Difference | |
| | population) | population) | (%) | p-value |
| Day 0 | 2.0 +/− 1.1 | 1.9 +/− 1.7 | +0.1 | 0.3411 |
| Day 28 | 2.1 +/− 1.3 | 1.7 +/− 1.6 | +0.4 | 0.3003 |
| Day 56 | 2.5 +/− 1.4 | 1.8 +/− 1.7 | +0.5316 | p < 0.001 |
| Day 0 to 56 | +0.4896 | −0.2 | +0.5311 | p = 0.0002 |
| | P < 0.0001 | | | |
| Day 28 to 56 | +0.4520 | −0.1 | +0.3587 | p < 0.0108 |
| | P < 0.0001 | | | |

TABLE 57
The changes of % of CD45+ cells in UP360 vs Placebo

| | UP360 | Placebo | | |
|---|---|---|---|---|
| | (% of cell | (% of cell | Difference | |
| | population) | population) | (%) | p-value |
| Day 0 | 33.3 +/− 7.3 | 34.4 +/− 8.1 | −1.1 | 0.5718 |
| Day 28 | 33.9 +/− 7.6 | 35.6 +/− 8.1 | −1.7 | 0.6606 |
| Day 56 | 32.5 +/− 8.2 | 37.1 +/− 8.0 | −3.761 | p = 0.0066 |
| Day 0 to 56 | −0.8 | 2.7 | −3.811 | p = 0.0175 |
| Day 28 to 56 | −1.4 | 1.5 | −3.220 | p = 0.0422 |

composition within groups. Mirroring the preclinical data, contemplated Aloe-based compositions, including UP360, comprising and, in some embodiments, consisting of polysaccharides and polyphenols showed statistically significant induction of the gamma delta T-cells. Based on the characteristics of this unique T-cell subpopulations described in the discussions, where these data clearly showed that the main activity of the Aloe-based composition in immune regulation, surveillance and homeostasis is as a result of the induction of these cells.

A similar but reverse pattern in the level of CD45+ cells was observed as a result of supplementation with the Aloe-based composition. As seen in the Table 57, the % CD45+ cells at day 56 was on average 3.761 lower for participants receiving UP360 compared to those receiving Placebo (p=0.0066). The change in % CD45+ cells from day 0 to day 56 was on average 3.811 lower for participants receiving UP360 compared to those receiving Placebo (p=0.0175). Similarly, the change in % CD45+ cells from day 28 to day 56 was on average 3.220 lower for participants receiving UP360 compared to those receiving Placebo (p=0.0442).

The Secondary Outcomes were the differences between UP360 and placebo at Day 28 and 56 in: 1. Number of confirmed COVID-19 infections; 2. Number of confirmed flu cases; 3. Impact of COVID-19 on quality of life assessed by the COVID-19 Impact on QoL Questionnaire; 4. Over-the-counter cold and flu medication use. The difference between UP360 and placebo at Day 56 in: 1. Number of hospitalizations due to COVID-19; 2. Number of hospitalizations due to flu.

The difference in change between UP360 and placebo from baseline to those measurements at Day 28 and 56 in: 1. Erythrocyte sedimentation rate (ESR) and C-reactive protein (CRP); 2. Hematology parameters: white blood cell (WBC) count with differential (neutrophils, lymphocytes, monocytes, eosinophils, basophils), reticulocyte count, red blood cell (RBC) count, hemoglobin, hematocrit, platelet count, RBC indices (mean corpuscular volume (MCV), mean corpuscular hemoglobin (MCH), mean corpuscular hemoglobin concentration (MCHC), and red cell distribution width (RDW); 3. Complement C3 and C4 proteins; 4. Mean global severity index, as measured by area under the curve (AUC) for the Modified Wisconsin Upper Respiratory Symptom Survey (WURSS)-24 daily symptom scores. 5. Mean symptom severity scores, as measured by AUC for the WURSS-24 daily severity symptom scores; 6. Number of well days (defined as days scored as 0 (not sick) for the question, “How sick do you feel today?”) as assessed by the Modified WURSS-24 Questionnaire; 7. Number of sick days (defined as days scored as any number from 1 through 7 (sick) for the question, “How sick do you feel today?”) as assessed by the Modified WURSS-24 Questionnaire; 8. Frequency of common upper respiratory tract infection (UTRI) symptoms as assessed by the Modified WURSS-24 Questionnaire; 9. Duration of common UTRI symptoms as assessed by the Modified WURSS-24 Questionnaire; 10. Severity of common UTRI symptoms as assessed by the Modified WURSS-24 Questionnaire; 11. Vitality and quality of life as assessed by the Vitality and Quality of Life (QoL) Questionnaire.

Contemplated methods further comprise methods for supporting healthy inflammatory response; maintaining healthy levels of Complement C3 and C4 proteins, cytokines and cytokine responses to infections; mitigating , regulating and maintaining TNF-α, IL-1β, IL-6, GM-CSF; IFN-α; IFN-γ; IL-1α; IL-1RA; IL-2; IL-4; IL-5; IL-7; IL-9; IL-10; IL-12 p′70; IL-13; IL-15; IL17A; IL-18; IL-21; IL-22; IL-23; IL-27; IL-31; TNF-β/LTA, CRP, and CINC3.

Samples were collected and stored for future analysis to analyze the difference in change between UP360 and placebo from baseline, at Day 28, and 56 in:
1. Cytokines (GM-CSF; IFN-α; IFN-γ; IL-1α; IL-1β; IL-1RA; IL-2; IL-4; IL-5; IL-6; IL-7; IL-9; IL-10; IL-12 p′70; IL-13; IL-15; IL17A; IL-18; IL-21; IL-22; IL-23; IL-27; IL-31; TNF-α; TNF-β/LTA 150)
2. High mobility group box 1 (HMGB1) protein, nuclear factor kappa B (NF-κB), nuclear factor erythroid 2-related factor 2 (Nrf-2)
3. Oxidative stress as assessed by 8-iso-prostaglandin F2α, catalase (CAT), glutathione peroxidase (GSH-Px), superoxide dismutase (SOD), malondialdehyde (MDA) and advanced glycation end-products (AGEs)
4. Hemagglutinin inhibition (HI) titers for specific strains of virus

In addition to the efficacy analysis, safety evaluations will be performed. 1. Clinical chemistry parameters: alanine aminotransferase (ALT), aspartate aminotransferase (AST), alkaline phosphatase (ALP), total bilirubin, creatinine, electrolytes (Na+, K+, Cl−), estimated glomerular filtration rate (eGFR), glucose; 2. Incidence of pre-emergent and post-emergent adverse events; 3. Vital signs (blood pressure (BP) and heart rate (HR)

### Example 53

### Clinical Proof-Of-Concept Study on Rapid Immune Modulating Effects of UP360

The goal for this clinical proof-of-concept study is to compare acute immune effects of a novel nutraceutical blend UP360 comprising polysaccharides and polyphenols made in Example 9 to a placebo. This data is important to verify immune related effects.

This clinical proof-of-concept study aims at documenting acute effects of consuming a test product through evaluation of immune cell activation, cell trafficking, and cytokine changes to pro- and anti-inflammatory cytokines, antiviral peptides, and restorative growth factors.

Data on immune cell trafficking and surveillance are collected. The testing show whether consuming the novel composition comprising polysaccharides and polyphenols to a rapid change in the alertness of the immune system to search for and attempt to eliminate microbial invaders, and to collaborate effectively between immune cell types.

For this clinical study, human subjects are tested following an established placebo-controlled, randomized, double-blinded, cross-over study design. Specifically, the study design has been used in previous clinical studies on immune modulating products on changes to lymphocyte trafficking, specifically stem cell subsets. Subjects are randomized to active or placebo prior to dosing a baseline sample is collected, after ingestion of investigational product blood samples are collected at 1, 2, 3 hours post dose. Subjects return to the clinic after a 7 day wash out and will take the opposite product in a crossover fashion, study procedures are repeated.

The test parameters we evaluate do not necessarily stay constant, even over a few hours, since they are related to people's metabolism, individual circadian rhythms, and other normal physiological parameters. Therefore, studies of this nature must include a placebo test day, allowing within-subject analysis of changes between the test days for each person. This very much strengthens the data analysis from this type of pilot study. In the absence of a placebo test day, we consider the data inconclusive since changes cannot be interpreted as being related to product intake.

The Primary outcome measure: Immune surveillance: Trafficking and activation of immune cells in vivo. The study is designed to show Rapid Immune Support by Immune surveillance and Immune alertness.

For this study 12 healthy subjects of either gender are enrolled after IRB-approved, written informed consent. The inclusion/exclusion profile for a study of this nature is not trivial, and each potential study participant is carefully evaluated prior to enrollment. To minimize anticipatory stress and apprehension during initial clinic visits for the study, each study participant must either have participated in previous studies at our facility or must attend a visit where we go through the study procedures, prior to a clinical study day.

The study includes Healthy adults; Age 18-75 years (inclusive); BMI between 18.0 and 34.9 (inclusive);willing to comply with study procedures, including: maintaining a consistent diet and lifestyle routine throughout the study, consistent habit of bland breakfasts on days of clinic visits, abstaining from exercise and nutritional supplements on morning of study visit, abstaining from use of coffee, tea, and soft drinks for at least one hour prior to a clinic visit; abstaining from music, candy, gum, computer/cell phone use, during clinic visits.

Excluded are subjects that meet these criteria: Previous major gastrointestinal surgery (absorption of test product may be altered) (minor surgery not a problem, including previous removal of appendix and gall bladder);Taking anti-inflammatory medications on a daily basis; Currently experiencing intense stressful events/life changes; Currently in intensive athletic training (such as marathon runners);Cancer during past 12 months; Chemotherapy during past 12 months; Currently treated with immune suppressant medication; Diagnosed with autoimmune disorders e.g. systemic lupus erythematosus, hemolytic anemia; Donation of blood during the study or within the 4 weeks prior to study start; Have received a cortisone shot within past 12 weeks; Immunization during last month; Currently taking anxiolytic, hypnotic, or anti-depressant prescription medication; Ongoing acute infections (including teeth, sinus, ear, etc.); Participation in another clinical trial study during this trial, involving an investigational product or lifestyle change; An unusual sleep routine (examples: working graveyard shift, irregular routine with frequent late nights, studying, partying); Unwilling to maintain a constant intake of supplements over the duration of the study; Women of childbearing potential: Pregnant, nursing, or trying to become pregnant; Known food allergies related to ingredients in active test product or placebo. Prescription medication will be evaluated on a case-by-case basis.

Consumable Test Products: Test products active UP360 comprising polysaccharides and polyphenols made in Example 9 and placebo will be provided. On each clinic day, immediately after the baseline blood draw, subjects are given a single dose of either the active test product UP360 or a placebo, in the presence of the clinic staff. Subjects consume the capsules with water and a few bland soda crackers to stimulate digestive function.

Explanation of the proposed clinical study procedures: In a clinical trial to monitor immune activating events, we expect a cascade of events, starting by activation of immune cells in the gut, systemic changes to cytokine levels, changes to immune cell trafficking (enhanced immune surveillance), followed by immune surveillance in tissue throughout the body, and possibly re-entry of activated immune cells back into the blood circulation.

Blood samples offer a convenient window into the immune events happening after a product is consumed. We do not have convenient windows into what may happen at the initial gut activation, but we envision this is like events in vitro. We do not have windows into tissue and thus cannot monitor downstream events after immune cells migrate from blood into tissue to scavenge for microbial invaders and perform innate and adaptive types of immune responses. Therefore, we mimic this by taking some of the blood samples and challenging the immune cells ex vivo (outside the body) with microbial mimetics.

The testing described aims to monitor rapid changes in the types and activation status of immune cells seen in the blood circulation. Increases versus decreases in numbers of immune cells in the blood is a measure of cellular trafficking in and out of the blood stream.

We are looking for subtle events, where any systematic changes observed in most of the study participants after consuming the same test product suggests immune activating events are induced. This is a good indication that a product has triggered increased immune awareness.

In immune surveillance the immune cells move in and out of tissue which can be measured by measuring cell numbers in circulating blood. In immune alertness we measure specific cells for function in circulating blood.

Immune cell trafficking and status of immune alertness

The analysis allows us to detect if consumption of a test product leads to rapid changes in cell numbers in the circulation, and/or activates cells in vivo. Freshly drawn blood samples are used for the testing of changes in immune cell numbers and activation status. The cells from each blood draw are assayed in triplicate.

Cells are stained with the T cell marker CD3 and the CD56 and CD57 markers, as well as the two activation markers CD69 and the interleukin-2 receptor CD25. This allows analysis of numbers of the following types of immune cells in the blood circulation at each time point in the study: CD3-negative, CD56-positive NK cells; CD3+CD56+ NKT cells; CD3+CD56− T lymphocytes; CD3−CD56-non-NK, non-T lymphocytes; CD3−CD57+ NK cells; CD3−CD56+CD57+ NK cells; Monocytes (identified by forward/side scatter profile).

During analysis, expression levels will be determined for the activation molecule CD69 and growth factor receptor CD25 on the surface of the cell populations listed above.

Note: Immune surveillance involves the constant recirculation of lymphocyte subsets, including NK and T cells. The trafficking shows a distinct circadian rhythm and is affected by a person's metabolic state. When comparing the acute effects of a consumable immune modulating product on immune surveillance, it is important to have a placebo control test day, to account for a given person's metabolic state.

It is feasible to add more flow cytometry panels. Budget options are included below. The additional panels include Add-on panel for numbers of gamma-delta (γδ) T cells (γδTCR+CD5−CD8−): CD3/ γδ T Cell Receptor; CD5; CD56—may be + or − on γδ T cells; CD69, CD25.

Add-on panel for numbers of B and T lymphocyte subsets, and CD45 isoform expression: CD4 T cell subset, CD8 T cell subset, CD19 B lymphocytes, CD45RA—expressed on naive T and B cells, CD45R0—expressed on activated and memory T and B cells.

### REFERENCES

1. Adomako-Bonsu A G, Chan S L, Pratten M, Fry J R. Antioxidant activity of Rosmarinic acid and its principal metabolites in chemical and cellular systems: Importance of physico-chemical characteristics. Toxicol In Vitro. 2017 April; 40:248-255. doi: 10.1016/j.tiv.2017.01.016. Epub 2017 Jan 22.
2. Al-Sereiti M, et al. Pharmacology of Rosemary (Rosmarinus officinalis Linn.) and its Therapeutic Potentials. Indian J Exp. Biol. 1999, 37, 124-130
3. Alvarez D F, Helm K, DeGregori J, Roederer M, Majka S. “Publishing flow cytometry data.” Am J Physiol Lung Cell Mol Physiol 298.2 (2010): L127-L130.
4. Amoah S K, Sandjo L P, Kratz J M, Biavatti M W. Rosmarinic Acid-Pharmaceutical and Clinical Aspects. Planta Med. 2016 Mar;82(5):388-406. doi: 10.1055/s-0035-1568274. Epub 2016 Feb 4.
5. Angelo L S, Banerjee P P, Monaco-Shawver L, Rosen J B, Makedonas G, Forbes L R, Mace E M, Orange J S. “Practical NK cell phenotyping and variability in healthy adults.” Immunol Res 62.3 (2015): 341-356.
6. Azman K F, Zakaria R. “D-Galactose-induced accelerated aging model: an overview.” Biogerontology 20.6 (2019): 763-782.
7. Barnes J, Anderson, L A, Philipson, J. D. Herbal Medicines, 3rd edition. London (UK): The Pharmaceutical Press; 2007.
8. BHP 1983: British Herbal Pharmacopoeia. Cowling (UK): British Herbal Medical Association; 1983.
9. Bianchi M E, Manfredi A A. High-mobility group box 1 (HMGB1) protein at the crossroads between innate and adaptive immunity. Immunol Rev. 2007 December; 220:35-46.
10. Blumenthal M, Goldberg A, Brinkmann J. Herbal Medicine: Expanded Commission E Monographs. Boston (MA): Integrative Medicine Communications; 2000.
11. Bonneville M, O'Brien R L, Born W K. Gammadelta T cell effector functions: a blend of innate programming and acquired plasticity. Nat Rev Immunol. 2010 July; 10(7):467-78.
12. Cao W, et al. Effects of rosmarinic acid on immunoregulatory activity and hepatocellular carcinoma cell apoptosis in H22 tumor-bearing mice. Korean J Physiol Pharmacol. 2019 November; 23(6):501-508. doi: 10.4196/kjpp.2019.23.6.501. Epub 2019 Oct. 24.
13. Cao W, Hu C, Wu L, Xu L, Jiang W. Rosmarinic acid inhibits inflammation and angiogenesis of hepatocellular carcinoma by suppression of NF-κB signaling in H22 tumor-bearing mice. J Pharmacol Sci. 2016; 132:131-137
14. Cheng S, Eliaz I, Lin J, Thyagarajan-Sahu A, Sliva D. Triterpenes from Poria cocos suppress growth and invasiveness of pancreatic cancer cells throμgh the downregulation of MMP-7. Int J Oncol. 2013; 42(6):1869-74.
15. Chow, J. T., Willamson, D. A., Yates, K. M., & Groux, W. J. (2005) Carbohydr. Res. 340, 1131-1142
16. Chuan-Xin Wu , Hang Sun, Qi Liu, Hui Guo, Jian-Ping Gong. LPS Induces HMGB1 Relocation and Release by Activating the NF-κB-CBP Signal Transduction Pathway in the Murine Macrophage-Like Cell Line RAW264.7. J Surg Res. 2012 Jun. 1; 175(1):88-100.
17. Colica C, Di Renzo L, Aiello V, De Lorenzo A, Abenavoli. Rosmarinic Acid as Potential Anti-Inflammatory Agent. L. Rev Recent Clin Trials. 2018;13(4):240-242. doi: 10.2174/157488711304180911095818.
18. Cuéllar M J, Giner R M, Recio M C, Just M J, Máñez S, Rios J L. Effect of the basidiomycete Poria cocos on experimental dermatitis and other inflammatory conditions. Chem Pharm Bull. 1997; 45: 492-494
19. Cuellar M J, Giner R M, Recio M C, Just M J, Máñez S, Rios J L. Two fungal lanostane derivatives as phospholipase A2 inhibitors. J Nat Prod 1996; 59: 977-979
20. Davis, R. H. (1997) Aloe Vera, a Scientific Approach, Vantage Press, New York, N.Y., pp 8-46
21. Derek C Angus, Lihong Yang, Lan Kong, John A Kellum, Russell L Delude, Kevin J Tracey, Lisa Weissfeld, GenIMS Investigators. Circulating High-Mobility Group Box 1 (HMGB1) Concentrations Are Elevated in Both Uncomplicated Pneumonia and Pneumonia With Severe Sepsis. Crit Care Med. 2007 April; 35(4):1061-7.
22. Feng Y L, Zhao Y Y, Ding F, Xi Z H, Tian T, Zhou F, Du X, Chen D Q, Wei F, Cheng X L, Lin R C. Chemical constituents of surface layer of Poria cocos and their pharmacological properties (I). Zhongguo Zhong Yao Za Zhi. 2013; 38(7): 1098-102.
23. Fuchs S M, Heinemann C, Schliemann-Willers S, Härtl H, Fluhr J W, Elsner P. Assessment of anti-inflammatory activity of Poria cocos in sodium lauryl sulphate-induced irritant contact dermatitis. Skin Res Technol. 2006; 12(4):223-7.
24. Gardner Z and McGuffin M. Astragalus membranaceus. American Herbal Products Association's Botanical Safety Handbook. CRC press LLC; 1997: 17-17
25. Gardner Z and McGuffin M. Wolfiporia cocos. American Herbal Products Association's Botanical Safety Handbook. CRC press LLC; 1997: 124-124
26. Gentile L F, Moldawer L L. HMGB1 as a therapeutic target for sepsis: it's all in the timing! Expert Opin Ther Targets. 2014 March; 18(3):243-5.
27. Giner-Larza E M, Má\ddot{n}ez S, Giner-Pons R M, Carmen Recio M, Rios J L. On the anti-inflammatory and anti-phospholipase A(2) activity of extracts from lanostane-rich species. J Ethnopharmacol. 2000; 73(1-2): 61-9.
28. Goh W, Huntinton N D. “Regulation of Murine Natural Killer Cell Development.” Front Immunol 8 (2017): 130.
29. Hamman, J. H. (2008) Molecules, 13, 1599-1616
30. Hauge S, Madhun A, Cox R J, Haaheim L R. “Quality and kinetics of the antibody response in mice after three different low-dose influenza virus vaccination strategies.” Clin Vaccine Immunol 14.8 (2007): 978-83.
31. Ighodaro O M, Akinloye O A. “First line defence antioxidants-superoxide dismutase (SOD), catalase (CAT) and glutathione peroxidase (GPX): Their fundamental role in the entire antioxidant defence grid.” Alexandria Journal of Medicine 54.4 (2018): 287-293.
32. Im S A, Lee Y R, Lee Y H, Lee M K, Park Y I, Lee S, Kim K, Lee C K. In vivo evidence of the immunomodulatory activity of orally administered Aloe vera gel. Arch Pharm Res. 2010 March; 33(3):451-6. doi: 10.1007/s12272-010-0315-1. Epub 2010 March 30.
33. Jeong J W, Lee H H, Han M H, Kim G Y, Hong S H, Park C, Choi Y H. Ethanol extract of Poria cocos reduces the production of inflammatory mediators by suppressing the NF-kappaB signaling pathway in lipopolysaccharide-stimulated RAW 264.7 macrophages. BMC Complement Altern Med. 2014; 14: 101
34. Jiang W L, Chen X G, Qu G W, et al. Rosmarinic acid protects against experimental sepsis by inhibiting proinflammatory factor release and ameliorating hemodynamics. Shock. 2009; 32(6): 608-613.
35. Kaminaga T, Yasukawa K, Takido M, Tai T, Nunoura Y. Inhibitory effects of Poria cocos on 12-O-tetradecanoylphorbol-13-acetate-induced edema and tumour promotion in mouse skin. Phytother Res 1996; 10: 581-584
36. Lee K Y, Jeon Y J. Polysaccharide isolated from Poria cocos sclerotium induces NF-kappaB/Rel activation and iNOS expression in murine macrophages. Int Immunopharmacol. 2003; 3(10-11):1353-62.
37. Lee K Y, You H J, Jeong H G, Kang J S, Kim H M, Rhee S D, Jeon Y J. Polysaccharide isolated from Poria cocos sclerotium induces NF-kappaB/Rel activation and iNOS expression throμgh the activation of p38 kinase in murine macrophages. Int Immunopharmacol. 2004; 4(8):1029-38.
38. Lee S M, Lee Y J, Yoon J J, Kang D G, Lee H S: Effect of Poria cocos on hypertonic stress-induced water channel expression and apoptosis in renal collecting duct cells. J Ethnopharmacol 2012, 141:368-376.
39. Leung AT and Foster S. Poria. Encyclopedia of Common Natural Ingredients Used in Food, Drμgs, and Cosmetics, 2nd edition. John Wiley& Sons, Inc. New York; 1996: 425-427
40. Li N, Liu X X, Hong M, Huang X Z, Chen H, Xu J H, Wang C, Zhang Y X, Zhong J X, Nie H, Gong Q. Sodium butyrate alleviates LPS-induced acute lung injury in mice via inhibiting HMGB1 release. Int Immunopharmacol. 2018 March; 56:242-248.
41. Li T H, Hou C C, Chang C L, Yang W C. Anti-Hyperglycemic Properties of Crude Extract and Triterpenes from Poria cocos. Evid Based Complement Alternat Med. 2011; 2011. pii: 128402.
42. Luan H, Kan Z, Xu Y, Lv C, Jiang W. Rosmarinic acid protects against experimental diabetes with cerebral ischemia: relation to inflammation response. J Neuroinflammation 2013; 10: 28.
43. Luo C, et al. A Review of the Anti-Inflammatory Effects of Rosmarinic Acid on Inflammatory Diseases. Front Pharmacol. 2020 Feb. 28; 11:153. doi: 10.3389/fphar.2020.00153.
44. Maria Entezari, Mohammad Javdan , Daniel J Antoine, et al. Inhibition of Extracellular HMGB1 Attenuates Hyperoxia-Induced Inflammatory Acute Lung Injury. Redox Biol. 2014 Jan. 20; 2:314-22.
45. Martins A, Han J, Kim S O. “The Multifaceted Effects of Granulocyte Colongy-Stimulating Factor in Immunomodulation and Potential Roles in Intestinal Immune Homeostasis.” IUBMB Life 62.8 (2010): 611-617.
46. Miao H, Hollenbaugh J A, Zand M S, Holden-Wiltse J, Mosmann T R, Perelson A S, Wu H, Topham D J. “Quantifying the early immune reponse and adaptive immune response kinetics in mice infected with influenza A virus.” J Virol 84.13 (2010): 6687-98.
47. Narni-Mancinelli E, Chaix J, Fenis A, Kerdiles Y M, Yessaad N, Reynders A, Gregoire C, Luche H, Ugolini S, Tomasello E, Walzer T, Vivier E. “Fate mapping analysis of lymphoid cells expressing the NKp46 cell surface receptor.” Proc Natl Acad Sci U.S.A. 108.45 (2011): 18324-18329.
48. Park Y H, Son I H, Kim B, Lyu Y S, Moon H I, Kang H W: Poria cocos water extract (PCW) protects PC12 neuronal cells from beta-amyloid-induced cell death throμgh antioxidant and antiapoptotic functions. Pharmazie 2009, 64:760-764.
49. Park, Y. I., & Lee, S. K. (2006) New Perspectives on Aloe, Springer, Science+Busines Media, LLC., New York, N.Y., pp 1-17
50. Park, Y. I., & Lee, S. K. (2006) New Perspectives on Aloe, Springer, Science+Busines Media, LLC., New York, N.Y., pp 7-62-a
51. Qiu, Z., Jones, K., Wylie, M., Jia, Q., & Orndorff, S. (2000) Planta Med. 66, 152-156
52. Raphael I, Nalawade S, Eagar T N, Forsthuber TG. “T cell subsets and their signature cytokines in autoimmune and inflammatory diseases.” Cytokine 74.1 (2015): 5-17.
53. Ribot J C, Lopes N, Silva-Santos B. γδ T cells in tissue physiology and surveillance. Nat Rev Immunol. 2021 April; 21(4):221-232.
54. Rios J L. Chemical constituents and pharmacological properties of Poria cocos. Planta Med. 2011; 77(7):681-91.
55. Ross, S. A., EISohly, M. A., & Wilkins, S. P. (1997) J. AOAC int. 80, 455-457
56. Scheckel K A, Degner S C, Romagnolo D F. Rosmarinic acid antagonizes activator protein-1-dependent activation of cyclooxygenase 2 expression in human cancer and nonmalignant cell lines. cell lines. J Nutr. 2008; 138(11): 2098-105.
57. Song E J, Espano E, Nam J H, Kim J, Shim K S, Shin E, Park Y I, Lee CK, Kim J K. “Adjuvanticity of Processed Aloe vera gel for Influenza Vaccination in Mice.” Immune Netw 20.4 (2020): e31.
58. Sun P, Kim Y, Lee H, Kim J, Han B K, Go E, Kwon S, Kang J G, You S, Kwon J. “Carrot Pomace Polysaccharide (CPP) Improves Influenza Vaccine Efficacy in Immunosuppressed Mice via Dendritic Cell Activation.” Nutrients 12.9 (2020): E2740.
59. Sun Y. Biological activities and potential health benefits of polysaccharides from Poria cocos and their derivatives. Int J Biol Macromol. 2014; 68: 131-4.
60. Swamp V, Ghosh J, Ghosh S, Saxena A, Basu A. Antiviral and anti-inflammatory effects of rosmarinic acid in an experimental murine model of Japanese encephalitis. Antimicrob Agents Chemother 2007; 51(9): 3367-70.
61. Torrado E, Cooper A M. “Cytokines in the balance of protection and pathology during mycobacterial infections.” Adv Exp Med Biol 783 (2013): 121-140.
62. Tseng J, Chang J G. Suppression of tumor necrosis factor-α, interleukin-1β, interleukin-6 and granulocyte-monocyte colony stimulating factor secretion from human monocytes by an extract of Poria cocos. Chin J Microbiol Immunol 1992; 1: 1-10
63. Vera E J, Chew Y V, Nicholson L, Burns H, Anderson P, Chen H T, Williams L, Keung K, Zanjani N T, Dervish S, Patrick E, Want X M, Yi S, Hawthorne W, Alexander S, O'Connell P J, Hu M. “Standardisation of flow cytometry for whole blood immunophenotyping of islet transplant and transplant clinical trial recipients.” PLoS One 14.5 (2019): e0217163.
64. Wang H, Bloom O, Zhang M, et al. HMG-1 as a late mediator of endotoxin lethality in mice. Science 1999; 285:248-51.
65. Wang W J, Cheng M H, Lin J H, Weng C S. Effect of a rosmarinic acid supplemented hemodialysis fluid on inflammation of human vascular endothelial cells. Braz J Med Biol Res 2017; 50(12):e6145.
66. Wu Y, et al. Effect of a polysaccharide from Poria cocos on humoral response in mice immunized by H1N1 influenza and HBsAg vaccines. Int J Biol Macromol. 2016 October; 91:248-57. doi: 10.1016/j.ijbiomac.2016.05.046. Epub 2016 May 13.
67. Xu J Wei K, Zhang G, Lei L, Yang D, Wang W, Han Q, Xia Y, Bi Y, Yang M, Li M. Ethnopharmacology, phytochemistry, and pharmacology of Chinese Salvia species: A review. J Ethnopharmacol. 2018 Oct. 28; 225:18-30. doi: 10.1016/j.jep.2018.06.029. Epub 2018 Jun. 20.
68. Yang H, Wang H, Tracey K J. HMG-1 rediscovered as a cytokine. Shock 2001; 15:247-53.
69. Yasukawa K, Kaminaga T, Kitanaka S, Tai T, Nunoura Y, Natori S, Takido. M. 3β-p-hydroxybenzoyldehydrotumulosic acid from Poria cocos, and its anti-inflammatory effects. Phytochemistry 1998; 48: 1357-1360
70. Yu S J, Tseng J. Fu-Ling, a Chinese herbal drug, modulates cytokine secretion by human peripheral blood monocytes. Int J Immunopharmacol 1996; 18: 37-44
71. Zhang W, et al. Adjuvant activity of PCP-II, a polysaccharide from Poria cocos, on a whole killed rabies vaccine. Virus Res. 2019 Sep;270:197638. doi: 10.1016/j .virusres.2019.06.001. Epub 2019 Jun. 4.
72. Zhao J, et al. Poria cocos polysaccharides attenuated ox-LDL-induced inflammation and oxidative stress via ERK activated Nrf2/HO-1 signaling pathway and inhibited foam cell formation in VSMCs. Int Immunopharmacol. 2020 March; 80:106173. doi: 10.1016/j.intimp.2019.106173. Epub 2020 Jan. 13
73. Zimmermann V. Rosemary as a Medicinal Plant and Wonder-Drug. A Report on the Medieval Drug Monographs. Sudhoffs Arch; 1980, 64:351-370.

1. A composition for regulation of immunity homeostasis, comprising a combination of an Aloe extract enriched for one or more polysaccharides; a Poria extract enriched for one or more polysaccharides; and a Rosemary extract enriched for one or more polyphenolic compounds.

2. The composition of claim 1, wherein the Aloe extract, or Poria extract or Rosemary extract in the composition is in a range of 1%-98% by weight of each extract with the optimized weight ratio of Aloe:Poria:Rosemary (APR) at 3:2:1 (50%:33.3%:16.7%) or 1:1:1 (33.3%:33.3%:33.3%) or 3:6:1 (30%:60%:10%).

3. The composition of claim 1, wherein the Aloe extract is whole leaf gel or inner leaf gel from Aloe vera or Aloe barbadense, the Poria extract is from mushroom or fruit body of Poria cocos or Wolfiporia extensa, and Rosemary extract is from leaf of Rosmarinus officinalis.

4. The composition of claim 1 wherein the Aloe extract comprises 0.01% to 99.9% of polysaccharides.

5. The composition of claim 1 wherein the Poria extract comprises 0.01% to 99.9% of polysaccharides.

6. The composition of claim 1 wherein the Rosemary extract comprises 0.01% to 99.9% of Rosmarinic acid.

7. The composition of claim 1, wherein the one or more polysaccharides from the Aloe extract is an acetylated polysaccharide or acemannan or any combination thereof.

8. The composition of claim 1, wherein the one or more polysaccharides from the Poria extract is a beta-glucan or a combination thereof.

9. The composition of claim 1, wherein the one or more polysaccharides are enriched from a plant species selected from the group consisting of or a combination thereof Aloe vera, Aloe barbadense, Aloe ferox, Aloe arborescens, Astragalus membranaceus, Ganoderma lucidum, Hordeum vulgare, Agaricus (A. blazei) subrufescens, Echinacea purpurea, Echinacea angustifolia, Aconitum Napellus (Monkshood), Sambucus nigra, Poria cocos Wolf, Wolfiporia extensa, Withania somnifera, Bupleurum falcatum, Glycyrrhiza spp, Panax quinquefolium, Panax ginseng C. A. Meyer, Korea red ginseng, Lentinula edodes (shiitake), Inonotus obliquus (Chaga mushroom), Lentinula edodes, Lycium barbarum, Lycium chinense, Phellinus linteus (fruit body), Trametes versicolor (fruit body), Cyamopsis tetragonolobus Cyamopsis tetragonolobus (guar gum), Trametes versicolor, Cladosiphon okamuranus Tokida, Undaria pinnatifida, mushrooms, seaweeds, yeasts, brown algae, Agave Nectar, brown seaweed, fermentable fiber, cereal, sea cucumber, agave, artichokes, asparagus, leeks, garlic, onions, rye, barley kernels, wheat, pears, apples, guavas, quince, plums, gooseberries, oranges and other citrus fruits.

10. The composition of claim 1, wherein the polyphenolic compounds are enriched from a plant species selected from the group consisting of or a combination thereof Melissa officinalis, Momordica balsamina, Mentha piperita, Perilla frutescens, Salvia officinalis, Teucrium scorodonia, Sanicula europaea, Coleus blumei, Thymus spp Hyptis verticillata, Lithospermum erythrorhizon, hornwort Anthoceros agrestis, Piper longum Linn, Coptis chinensis Franch, Angelica sinensis (Oliv.) Diels, Toxicodendron vernicifluum, Glycyrrhiza glabra, Glycyrrhiza uralensis, Curcuma longa, Salvia Rosmarinus, Rosmarinus officinalis, Zingiber officinalis, Polygala tenuifolia, Humulus lupulus, Lonicera Japonica, Salvia officinalis L., Centella asiatica, Boswellia carteri, Mentha longifolia, Picea crassifolia, Citrus nobilis Lour, Citrus aurantium L. Camellia sinensis L. Pueraria mirifica, Pueraria lobata, Glycine max, Capsicum species, Fallopia japonica, tea, tomato, cruciferous vegetables, grapes, blueberries, raspberries, mulberries, apple, chili peppers.

11. The composition of claim 1, wherein the polyphenolic compounds comprise Rosmarinic acid, conjugated catechins such as EGCG, ECG, epigallocatechin, oroxylin, Kaempferol, genistein, quercetin, Butein, Luteolin, chrysin, Apigenin, curcumin, resveratrol, capsaicin, glomeratose A, 6-shogaol, gingerol, berberine, Piperine or a combination thereof.

12. The composition of claim 1, wherein the polysaccharides and polyphenols are enriched from a plant part or a fungus selected from the group consisting of leaves, bark, trunk, trunk bark, stem, stem bark, twigs, tubers, root, rhizome, root bark, bark surface, young shoots, seed, fruit, fruitbody, mushroom, androecium, gynoecium, calyx, stamen, petal, sepal, carpel (pistil), flower, or any combination thereof.

13. The composition of claim 1, wherein the Aloe extract, the Poria extract and the Rosemary extract in the composition are extracted with any suitable solvent, including supercritical fluid of CO₂, water, methanol, ethanol, acetone, alcohol, a water-mixed solvent or a combination thereof.

14. The composition of claim 1, wherein the polysaccharides are enriched individually and/or in combination by solvent precipitation, ultrafiltration, enzyme digestion, column chromatograph with silica gel, XAD, HP20, LH20, C-18, alumina oxide, polyamide, CG161, and size exclusion column resins.

15. The composition of claim 1, wherein one or more polyphenols are enriched individually or in combination by solvent partition, precipitation, distillation, evaporation, ultrafiltration, column chromatograph with silica gel, XAD, HP20, LH2O, C-18, alumina oxide, polyamide, size exclusion column and CG161 resins.

16. The composition of claim 1, wherein the composition further comprises a pharmaceutically or nutraceutically acceptable active, adjuvant, carrier, diluent, or excipient, and wherein the pharmaceutical or nutraceutical formulation comprises from about 0.1 weight percent (wt %) to about 99.9 wt % of active compounds.

17. The composition of claim 16, wherein the active, adjuvant, excipient or carrier comprises Cannabis sativa oil or CBD/THC, turmeric extract or curcumin, terminalia extract, willow bark extract, Devil's claw root extract, cayenne pepper extract or capsaicin, Prickly Ash bark extract, philodendra bark extract, hop extract, Boswellia extract, rose hips extract, green tea extract, Sophora extract, Mentha or Peppermint extract, ginger or black ginger extract, green tea or grape seed polyphenols, Omega-3 or Omega-6 Fatty Acids, fish oil, Krill oil, gamma-linolenic acid, citrus bioflavonoids, Acerola concentrate, astaxanthin, pycnogenol, vitamin C, vitamin D, vitamin E, vitamin K, vitamin B, vitamin A, L-lysine, calcium, manganese, zinc, mineral amino acid chelate(s), amino acid(s), boron and boron glycinate, silica, probiotics, Camphor, Menthol, calcium-based salts, silica, histidine, copper gluconate, CMC, maltodextrin, beta-cyclodextrin, cellulose, dextrose, saline, water, oil, shark and bovine cartilage, or a combination thereof.

18. The composition of claim 1, wherein the composition is formulated as a tablet, hard capsule, soft gel capsule, powder, or granule, compressed tablet, pill, gummy, chewing gum, sashay, wafer, bar, or liquid form, tincture, aerial spread, semi solid, semi liquid, solution, emulsion, cream, lotion, ointment, gel base or like form.

19. A method for treating, managing, promoting regulation of immunity homeostasis in a mammal, comprising administering an effective amount of a composition from 0.01 mg/kg to 500 mg/kg body weight of the mammal.

20. The method of claim 19, wherein the composition comprises a combination of an Aloe extract enriched for one or more polysaccharides; a Poria extract enriched for one or more polysaccharides; and a Rosemary extract enriched for one or more polyphenolic compounds.

21. The method of claim 19, wherein administering the composition is selected from the group comprising oral administration, topical administration, suppository administration, intravenous administration, intradermic administration, intragastric administration, intramuscular administration, intraperitoneal administration, and intravenous administration.

22. The method of claim 19, including maintaining immune homeostasis by optimizing or balancing the immune response; improving aging and immune organ senescence compromised immunity; preventing chronic inflammation and inflammation-compromised immunity; helping to maintain a healthy immune response to influenza vaccination or COVID-19 vaccination; helping to maintain a healthy immune function against virus infection and bacterial infections; protecting the immune system from oxidative stress damage induced by air pollution of a mammal.

23. The method of claim 19, further comprising a method for regulating HMGB1 as endogenous or exogenous response assault triggers and shifting host immune response to restore homeostasis by inhibition of HMGB1 release or counteract its action as targeting HMGB1 active or passive release by blocking cytoplasm translocation, or by blocking vesicle mediated release; or inhibiting intramolecular disulfide bond formation in the nucleus; targeting HMGB1 directly upon release and neutralize its effect; blocking HMGB1 pattern recognizing receptors such as Toll-like Receptor (TLR)-2/4/7/9 and receptor for advanced glycation end products (RAGE) or inhibiting their signal transductions; changing the physiochemical microenvironment and preventing formation of HMGB1 tetramer and interfere the binding affinity of HMGB1 to TLR and RAGE, preventing cluster formation or self-association of HMGB1.

24. The method of claim 19, further comprising a method for supporting healthy inflammatory response; maintaining healthy levels of Complement C3 and C4 proteins, cytokines and cytokine responses to infections; mitigating , regulating and maintaining TNF-α, IL-1β, IL-6, GM-CSF; IFN-α; IFN-γ; IL-1α; IL-1RA; IL-2; IL-4; IL-5; IL-7; IL-9; IL-10; IL-12 p70; IL-13; IL-15; IL17A; IL-18; IL-21; IL-22; IL-23; IL-27; IL-31; TNF-β/LTA, CRP, and CINC3.

25. The method of claim 19, further comprising controlling oxidative response and alleviating oxidative stress; augmenting antioxidant capacity by increasing catalase (CAT), glutathione peroxidase (GSH-Px), superoxide dismutase (SOD), and Nrf2; reducing or maintaining malondialdehyde (MDA), 8-iso-prostaglandin F2α, and advanced glycation end-products (AGEs); neutralizing reactive oxygen species; protecting UV and chemical oxidative stress caused DNA damage of a mammal.

26. The method of claim 19, further comprising improving innate immunity; improving adaptive immunity; increasing the activity and count of the white blood cells, enhancing Natural Killer (NK) cell function; increasing, regulating, maintaining the counts of T and B lymphocytes, neutrophils, lymphocytes, monocytes, eosinophils, basophils; increasing CD3+, CD3−CD56+ NK cells, CD3+CD56+ NKT cells, CD3+CD56− T lymphocytes, CD3−CD56− non-NK, non-T lymphocytes, CD3−CD57+ NK cells, CD3−CD56+CD57+ NK cells, CD4+ NKp46+ Natural Killer cells, TCRγδ+ Gamma delta T cells, and CD4+TCRγδ+ Helper Gamma delta T cells and CD8+ cell counts; regulating CD45+ cells, CD45RA naïve T and B cells,CD45R0 activated and memory T and B cells; protecting and promoting macrophage phagocytic activity; and supporting or promoting normal antibody IgG, IgM, IgA production, hemagglutinin inhibition (HI) titers for specific strains of virus of a mammal.

27. The method of claim 19, further comprising maintaining healthy pulmonary microbiota or symbiotic system in respiratory organs; maintaining lung cleansing and detox capability;
protecting lung structural integrity and oxygen exchanging capacity; maintaining respiratory passages and enhancing oxygen absorption capacity of alveoli; protecting normal healthy lung function from virus infection, bacterial infections, smoking and air pollution; mitigating oxidative stress caused pulmonary damage; and promoting microcirculation of the lung and protecting normal coagulation function of a mammal.

28. The method of claim 19, further comprising relieving or reducing cold/flu-like symptoms comprising body aches, sore throat, cough, minor throat and bronchial irritation, nasal congestion, sinus congestion, sinus pressure, runny nose, sneezing, loss of smell, loss of taste, muscle sore, headache, fever and chills; helping loosen phlegm (mucus) and thin bronchial secretions to make coughs more productive; reducing severity of bronchial irritation; reducing severity of lung damage or edema or inflammatory cell infiltration caused by virus infection, microbial infection and air pollution; supporting bronchial system and comfortable breathing through the cold/flu or pollution seasons; preventing or treating lung fibrosis; reducing duration or severity of common cold/flu; reducing severity or duration of virus and bacterial infection of respiratory system; preventing, or treating or curing respiratory infections caused by virus, microbial, and air pollutants; managing or treating or preventing, or reversing the progression of respiratory infections; and managing or treating or preventing, or reversing the progression of pneumonia, promoting and strengthening and rejuvenating the repair and renewal function of lung and the entire respiratory system of a mammal.

29. A composition for maintenance of immunity homeostasis by regulating HMGB1, comprising a combination of one or more polysaccharides and one or more polyphenolic compounds, wherein the composition modulates HMGB1 by inhibition of HMGB1 release or counteract its action as targeting HMGB1 active or passive release by blocking cytoplasm translocation, or by blocking vesicle mediated release; or inhibiting intramolecular disulfide bond formation in the nucleus; or targeting HMGB1 directly upon release and neutralize its effect; or blocking HMGB1 pattern recognizing receptors such as Toll-like Receptor (TLR)-2/4/7/9 and receptor for advanced glycation end products (RAGE) or inhibiting their signal transductions; or changing the physiochemical microenvironment and preventing formation of HMGB1 tetramer and interfere the binding affinity of HMGB1 to TLR and RAGE; or preventing cluster formation or self-association of HMGB 1.

30. The composition of claim 29, wherein the polysaccharides and phenolic compounds in the composition is in a range of 1%: 99% and 99% :1% by weight of each type of compound.

31. The composition of claim 29, wherein the one or more polysaccharides are enriched from a plant species comprising Aloe vera, Aloe barbadense, Aloe ferox, Aloe arborescens, Astragalus membranaceus, Ganoderma lucidum, Hordeum vulgare, Agaricus (A. blazei) subrufescens, Echinacea purpurea, Echinacea angustifolia, Aconitum Napellus (Monkshood), Sambucus nigra, Poria cocos Wolf, Wolfiporia extensa, Withania somnifera, Bupleurum falcatum, Glycyrrhiza spp, Panax quinquefolium, Panax ginseng C. A. Meyer, Korea red ginseng, Lentinula edodes (shiitake), Inonotus obliquus (Chaga mushroom), Lentinula edodes, Lycium barbarum, Lycium chinense, Phellinus linteus (fruit body), Trametes versicolor (fruit body), Cyamopsis tetragonolobus Cyamopsis tetragonolobus (guar gum), Trametes versicolor, Cladosiphon okamuranus Tokida, Undaria pinnatifida, mushrooms, seaweeds, yeasts, brown algae, Agave Nectar, brown seaweed, fermentable fiber, cereal, sea cucumber, agave, artichokes, asparagus, leeks, garlic, onions, rye, barley kernels, wheat, pears, apples, guavas, quince, plums, gooseberries, oranges and other citrus fruits, or a combination thereof.

32. The composition of claim 29, wherein the polyphenolic compounds are enriched from a plant species comprising Melissa officinalis, Momordica balsamina, Mentha piperita, Perilla frutescens, Salvia officinalis, Teucrium scorodonia, Sanicula europaea, Coleus blumei, Thymus spp., Hyptis verticillata, Lithospermum erythrorhizon, hornwort Anthoceros agrestis, Piper longum Linn, Coptis chinensis Franch, Angelica sinensis (Oliv.) Diels, Toxicodendron vernicifluum, Glycyrrhiza glabra, Glycyrrhiza uralensis, Curcuma longa, Salvia Rosmarinus, Rosmarinus officinalis, Zingiber officinalis, Polygala tenuifolia, Morus alba, Humulus lupulus, Lonicera Japonica, Salvia officinalis L., Centella asiatica, Boswellia carteri, Mentha longifolia, Picea crassifolia, Citrus nobilis Lour, Citrus aurantium L. Camellia sinensis L. Pueraria mirifica, Pueraria lobata, Glycine max, Capsicum species, Fallopia japonica, tea, tomato, cruciferous vegetables, grapes, blueberries, raspberries, mulberries, apple, chili peppers, or a combination thereof.

33. The composition of claim 29, wherein the polyphenolic compounds comprise rosmarinic acid, conjugated catechins such as EGCG, ECG, epigallocatechin etc. Oroxylin, Kaempferol, genistein, quercetin, Butein, Luteolin, chrysin, Apigenin, curcumin, resveratrol, capsaicin, glomeratose A, 6-shogaol, gingerol, berberine, Piperine, or a combination thereof.

34. The composition of claim 29, wherein the polysaccharides and polyphenolic compounds are enriched from a plant part or a fungus selected from the group consisting of leaves, bark, trunk, trunk bark, stem, stem bark, twigs, tubers, root, rhizome, root bark, bark surface, young shoots, seed, fruit, fruitbody, mushroom, androecium, gynoecium, calyx, stamen, petal, sepal, carpel (pistil), flower, or any combination thereof.

35. The composition of claim 29, wherein the polysaccharides and polyphenolic compounds in the composition are extracted from biomasses with any suitable solvent, including supercritical fluid of CO₂, water, methanol, ethanol, alcohol, a water-mixed solvent or a combination thereof.

36. The composition of claim 29, wherein the polysaccharides are enriched individually or in combination by solvent precipitation, ultrafiltration, enzyme digestion, column chromatograph with silica gel, XAD, HP20, LH20, C-18, alumina oxide, polyamide, size exclusion column, and CG161 resins.

37. The composition of claim 29, wherein one or more polyphenolic compounds are enriched individually or in combination by solvent partition, precipitation, ultrafiltration, distillation, evaporation, column chromatograph with silica gel, XAD, HP20, LH20, C-18, alumina oxide, polyamide and CG161 resins.

38. The composition of claim 29, wherein the composition further comprises a pharmaceutically or nutraceutically acceptable active, adjuvant, carrier, diluent, or excipient, wherein the pharmaceutical or nutraceutical formulation comprises from about 0.1 weight percent (wt %) to about 99.9 wt% of active compounds.

39. The composition of claim 38, wherein the active, adjuvant, excipient or carrier is selected from one or more of Cannabis sativa oil or CBD/THC, turmeric extract or curcumin, terminalia extract, willow bark extract, Devil's claw root extract, cayenne pepper extract or capsaicin, Prickly Ash bark extract, philodendra bark extract, hop extract, Boswellia extract, rose hips extract, green tea extract, Sophora extract, Mentha or Peppermint extract, ginger or black ginger extract, green tea or grape seed polyphenols, Omega-3 or Omega-6 Fatty Acids, Krill oil, gamma-linolenic acid, citrus bioflavonoids, Acerola concentrate, astaxanthin, pycnogenol, vitamin C, vitamin D, vitamin E, vitamin K, vitamin B, vitamin A, L-lysine, calcium, manganese, Zinc, mineral amino acid chelate(s), amino acid(s), boron and boron glycinate, silica, probiotics, Camphor, Menthol, calcium-based salts, silica, histidine, copper gluconate, CMC, maltodextrin, beta-cyclodextrin, cellulose, dextrose, saline, water, oil, shark and bovine cartilage, or a combination thereof

40. The composition of claim 29, wherein the composition is formulated as a tablet, hard capsule, soft gel capsule, powder, or granule, compressed tablet, pill, gummy, chewing gum, sashay, wafer, bar, or liquid form, tincture, aerial spread, semi solid, semi liquid, solution, emulsion, cream, lotion, ointment, or gel base.

41. The composition of claim 29, wherein the composition has a route of administration comprising oral administration, topical administration, suppository administration, intravenous administration, intradermic administration, intragastric administration, intramuscular administration, intraperitoneal administration, and intravenous administration.

42. A method for treating, managing, promoting regulation of immunity homeostasis in a mammal, comprising administering an effective amount of the composition of claim 29 in an amount from 0.01 mg/kg to 500 mg/kg body weight of the mammal.

43. A method for maintaining immune homeostasis by optimizing or balancing the immune response; helping to maintain a healthy immune function against virus infection and bacterial infections; protecting immune system from oxidative stress damage induced by air pollution; protecting normal healthy lung function from virus infection, bacterial infections and air pollution comprising administering an effective amount of the composition of claim 29 in an amount from 0.01 mg/kg to 500 mg/kg body weight of the mammal.

44. A method for regulating HMGB1 as endogenous or exogenous response assault triggers and shifting host immune response to restore homeostasis comprising administering an effective amount of the composition of claim 29 in an amount from 0.01 mg/kg to 500 mg/kg body weight of the mammal.

45. A method for supporting healthy inflammatory response; maintaining healthy level of Complement C3 and C4 proteins, cytokines and cytokine responses to infections; mitigating, regulating and maintaining TNF-α, IL-1β, IL-6, GM-CSF; IFN-α; IFN-β; IL-1α; IL-1RA; IL-2; IL-4; IL-5; IL-7; IL-9; IL-10; IL-12 p70; IL-13; IL-15; IL17A; IL-18; IL-21; IL-22; IL-23; IL-27; IL-31; TNF-β/LTA, CRP, and CINC3 comprising administering an effective amount of the composition of claim 29 in an amount from 0.01 mg/kg to 500 mg/kg body weight of the mammal.

46. A method of controlling oxidative response and alleviating oxidative stress; augmenting antioxidant capacity by increasing catalase (CAT), glutathione peroxidase (GSH-Px), superoxide dismutase (SOD), and Nrf2; reducing or maintaining malondialdehyde (MDA), 8-iso-prostaglandin F2α, and advanced glycation end-products (AGEs); neutralizing reactive oxygen species; protecting UV and chemical oxidative stress from causing DNA damage comprising administering an effective amount of the composition of claim 29 in an amount from 0.01 mg/kg to 500 mg/kg body weight of the mammal.

47. A method for improving innate immunity; improving adaptive immunity; increasing the activity and count of the white blood cells, enhancing Natural Killer (NK) cell function;
increasing, regulating, maintaining the counts of T and B lymphocytes, neutrophils, lymphocytes, monocytes, eosinophils, basophils; increasing CD3+, CD3−CD56+ NK cells, CD3+CD56+ NKT cells, CD3+CD56− T lymphocytes, CD3−CD56− non-NK, non-T lymphocytes, CD3−CD57+ NK cells, CD3−CD56+CD57+ NK cells, CD4+ NKp46+ Natural Killer cells, TCRγδ+ Gamma delta T cells, and CD4+TCRγδ+ Helper Gamma delta T cells and CD8+ cell counts; regulating CD45+ cells, CD45RA naïve T and B cells, CD45R0 activated and memory T and B cells; protecting and promoting macrophage phagocytic activity; supporting or promoting normal antibody IgG, IgM, IgA production, hemagglutinin inhibition (HI) titers for specific strains of virus comprising administering an effective amount of the composition of claim 29 in an amount from 0.01 mg/kg to 500 mg/kg body weight of the mammal.

48. A method of maintaining healthy pulmonary microbiota or symbiotic system in respiratory organs; maintaining lung cleanse and detox capability; protecting lung structure integrity and oxygen exchanging capacity; maintaining respiratory passages and enhancing oxygen absorption capacity of alveoli; mitigating oxidative stress caused pulmonary damage;
promoting microcirculation of the lung and protecting normal coagulation function comprising administering an effective amount of the composition of claim 29 in an amount from 0.01 mg/kg to 500 mg/kg body weight of the mammal.

49. A method of relieving or reducing cold or flu-like symptoms comprising body aches, sore throat, cough, minor throat and bronchial irritation, nasal congestion, sinus congestion, sinus pressure, runny nose, sneezing, loss of smell, loss of taste, muscle sore, headache, fever and chills; helping loosen phlegm (mucus) and thin bronchial secretions to make coughs more productive; reducing severity of bronchial irritation; reducing severity of lung damage or edema or inflammatory cell infiltration caused by virus infection, microbial infection and air pollution; supporting bronchial system and comfortable breathing through the cold/flu or pollution seasons; preventing or treating lung fibrosis; reducing duration or severity of common cold/flu; reducing severity or duration of virus and bacterial infection of respiratory system; preventing, or treating or curing respiratory infections caused by virus, microbial, and air pollutants; managing or treating or preventing, or reversing the progression of respiratory infections; and managing or treating or preventing, or reversing the progression of pneumonia, promoting and strengthening and rejuvenating the repair and renewal function of lung and the entire respiratory system of a mammal, comprising administering an effective amount of the composition of claim 29 in an amount from 0.01 mg/kg to 500 mg/kg body weight of the mammal.
